(19) United States
(12) Patent Application Publication
(10) Pub. No.: US 2026/0232820 A1
(43) Pub. Date: Aug. 13, 2026

(54) USE OF IMMUNOMODULATION METHODS IN COMBINATION WITH CYTOKINE FUSIONS FOR DISEASE TREATMENT

(71) Applicant: Medicenna Therapeutics Inc., Toronto (CA)

(72) Inventor: Fahar Merchant, Vancouver (CA)

(21) Appl. No.: 19/474,659

(22) PCT Filed: Apr. 13, 2024
(86) PCT No.: PCT/IB2024/053637

§ 371 Date: Oct. 10, 2025

Related U.S. Application Data
(60) Provisional application No. 63/496,364, filed on Apr. 14, 2023.

(51) Int. Cl. A61K 47/64; A61K 38/20; A61P 35/00

(52) U.S. Cl. CPC A61K 47/642; A61K 38/2013; A61P 35/00

(57) ABSTRACT

Provided herein are methods of treating a cancer comprising administering: a) an IL-4 receptor targeted cargo protein or an IL-13 receptor targeted cargo protein; and b) immunomodulatory agent (e.g., an IL-2 mutein or IL-2 mutein fusion protein described herein). Such combination therapies are particularly useful for the treatment of cancers that overexpress IL-4R or IL-13Rα2. Also provided herein are composition for use with the subject methods. Aspects of the methods and compositions are further detailed below.

### CROSS-REFERENCE TO RELATED APPLICATIONS

This application is a U.S. national phase of PCT Application No. PCT/IB24/53637, filed Apr. 13, 2024, which claims priority to U.S. Provisional Application No. 63/496,364, filed Apr. 14, 2023, which is herein incorporated by reference in its entirety.

### SEQUENCE LISTING

The instant application contains a Sequence Listing which has been submitted electronically in XML format and is hereby incorporated by reference in its entirety. Said XML copy, created on Oct. 31, 2025, is named 117802-5018-US.xml and is 416,692 bytes in size.

### BACKGROUND

IL-4R, is an ideal but under-exploited target for the development of cancer therapeutics, as it is frequently and intensely expressed on a wide variety of human carcinomas. Expression levels of IL-4R are low on the surface of healthy and normal cells, but increase several-fold on cancer cells. A majority of cancer biopsy and autopsy samples from adult and pediatric central nervous system (CNS) tumors, including recurrent GB biopsies, have been shown to over-express the IL-4R. There is little or no IL-4R expression in normal adult and pediatric brain tissue (Joshi, et a., 2001; see Table 2 of the reference).

Interleukin-13 receptor subunit alpha-1 (IL-13Ral, CD213A1), together with IL-4R, can form a functional receptor for IL13 (type II receptor), which will induce downstream signaling and promote tumor Myeloid Derived Suppressor Cells (MDSCs) and M2a Tumor Associated Macrophages (TAM). Blocking signals from type II receptor will lead to inhibit immune suppressive capabilities and therefore inhibit tumor growth and progression.

Interleukin-13 receptor subunit alpha-2 (IL-13Rα2, CD213A), a high-affinity membrane receptor of the anti-inflammatory Th2 cytokine IL-13, is overexpressed in a variety of solid tumors and is correlated with poor prognosis in glioblastoma, colorectal cancer, adrenocortical carcinoma, pancreatic cancer, and breast cancer. While initially hypothesized as a decoy receptor for IL-13-mediated signaling, recent evidence demonstrates IL-13 can signal through IL-13Rα2 in human cells. In addition, expression of IL-13Rα2 and IL-13Rα2-mediated signaling has been shown to promote tumor proliferation, cell survival, tumor progression, invasion, and metastasis. Given its differential expression in tumor versus normal tissue, IL-13Rα2 is an attractive immunotherapy target, as both a targetable receptor and an immunogenic antigen.

Thus, there is a need for cancer treatments and therapeutics that target IL-4R and IL-13 receptors.

### BRIEF SUMMARY

Provided herein are methods of treating a cancer comprising administering: a) an IL-4 receptor targeted cargo protein or an IL-13 receptor targeted cargo protein; and b) immunomodulatory agent (e.g., an IL-2 mutein or an immune checkpoint inhibitor described herein). Such combination therapies are particularly useful for the treatment of cancers that overexpress IL-4R or IL-13 receptors. Also provided herein are composition for use with the subject methods. Aspects of the methods and compositions are further detailed below.

In one aspect, provided herein is a method of treating a cancer in a patient in need thereof comprising administering to the patient: a) an IL-4 receptor targeted cargo protein or an IL-13 receptor targeted cargo protein; and b) an immunomodulatory agent, wherein the IL-4 receptor targeted cargo protein and/or the IL-13 receptor targeted cargo protein comprises an IL-4, an IL-13, or IL-4 mutein, or an IL-13 mutein linked to a cargo moiety, and wherein the cargo moiety is capable of inhibiting cancer cell growth or killing cancer cells.

In some embodiments, the IL-4 or IL-4 mutein is selected from the IL-4 muteins in Table 6. In some embodiments, the IL-4 receptor targeted cargo protein has the amino acid sequence of SEQ ID NO:1057.

In some embodiments, the IL-13 or IL-13 mutein is selected from the IL-13 muteins disclosed in Tables 5, 7, and 8. In some embodiments, the IL-13 mutein has the amino acid sequence of SEQ ID NO: 105, 106, 110, 118, or 159.

In some embodiments, the immunomodulatory agent is an IL-2 mutein or IL-2 mutein fusion protein. In some embodiments, the immunomodulatory agent is selected from an immune checkpoint inhibitor and a fusion protein disclosed in Tables 4 and 8. In some embodiments, the IL-2 mutein or IL-2 mutein fusion protein is selected from IL-2 mutein or IL-2 mutein fusion protein in Tables 2-4. In some embodiments, the IL-2 mutein fusion protein has the amino acid of SEQ ID NO:31.

In some embodiments, the cargo moiety is selected from Pseudomonas exotoxin, Aerolysin, Proaerolysin, Bouganin, a Cholera toxin, and Ribonuclease A. In some embodiments, the cargo moiety is Pseudomonas exotoxin.

In some embodiments, the cancer is associated with IL-4R overexpression or IL-13Rα2 overexpression. In some embodiments, the cancer is refractory to immune-checkpoint inhibition.

In some embodiments, the cancer is selected from the group consisting of sarcoma, carcinoma, head and neck cancer, glioblastoma, bladder cancer, oral cancer, mesothelioma, pancreatic cancer, liver cancer, colorectal cancer, pulmonary cancer, cutaneous, lymphoid, gastrointestinal cancer, prostate cancer, ovarian cancer, breast cancer, basal-like breast tumor, endometrial cancer, multiple myeloma, melanoma, lymphoma, lung cancer (including small cell lung cancer), kidney cancer, gastric cancer, brain cancer, and CNS tumors.

In some embodiments, the IL-4 receptor targeted cargo protein or IL-13 receptor targeted cargo protein is administered before the immunomodulatory agent. In certain embodiments, the IL-4 receptor targeted cargo protein or IL-13 receptor targeted cargo protein is administered after the immunomodulatory agent. In some embodiments, the IL-4 receptor targeted cargo protein or IL-13 receptor targeted cargo protein is administered contemporaneously with the immunomodulatory agent.

In another aspect, provided herein is a composition comprising: a) an IL-4 receptor targeted cargo protein or IL-13 receptor targeted cargo protein; and b) an immunomodulatory agent, wherein the IL-4 receptor targeted cargo protein or IL-13 receptor targeted cargo protein comprises an IL-4, an IL-13, an IL-4 mutein, or an IL-13 mutein linked to a cargo moiety, and wherein the cargo moiety is capable of inhibiting cancer cell growth or killing cancer cells.

In some embodiments, the IL-4 or IL-4 mutein is selected from the IL-4 muteins in Table 6. In some embodiments, the IL-4 receptor targeted cargo protein has the amino acid sequence of SEQ ID NO:1057.

In some embodiments, the IL-13 or IL-13 mutein is selected from the IL-13 muteins disclosed in Tables 5, 7, and 8. In some embodiments, the IL-13 mutein has the amino acid sequence of SEQ ID NO:105, 106, 110, 118, or 159.

In some embodiments, the immunomodulatory agent is an IL-2 mutein or IL-2 mutein fusion protein. In some embodiments, the immunomodulatory agent is selected from an immune checkpoint inhibitor and a fusion protein disclosed in Tables 4 and 8. In some embodiments, the IL-2 mutein or IL-2 mutein fusion protein is selected from IL-2 mutein or IL-2 mutein fusion protein in Tables 2-4. In some embodiments, the IL-2 mutein fusion protein has the amino acid of SEQ ID NO:31.

In some embodiments, the cargo moiety is selected from Pseudomonas exotoxin, Aerolysin, Proaerolysin, Bouganin, a Cholera toxin, and Ribonuclease A. In some embodiments, the cargo moiety is Pseudomonas exotoxin.

### BRIEF DESCRIPTION OF THE DRAWINGS

FIG. 1: (SEQ ID Nos. 1064-1067) Provides examples of IgG1, IgG2, IgG3, and IgG4 sequences.

FIG. 2: Fc-MDNA413 demonstrates tumor growth similar to vehicle control and MDNA19 exhibits moderate tumor growth inhibition in the TRAMP-C1 prostate tumor model. However, the combination of Fc-MDNA413 and MDNA19 shows superior tumor growth inhibition compared to either of the agents alone.

FIG. 3: Graphs of dose responses in A375 and U87 Cells. Percent Viability was normalized wherein 0% was defined as smallest mean and 100% was defined as largest mean in each data set. It was then plotted as a function of the construct concentration (pM). The average viability of the positive control wells is presented as a dotted line for bar graphs.

FIG. 4: Graphs of dose responses in EMT6-IL13Rα2 and EMT6 wild type Cells. Percent Viability was normalized wherein 0% was defined as smallest mean and 100% was defined as largest mean in each data set for EMT6-IL13Rα2. It was then plotted as a function of the construct concentration (pM). For EMT6 wild type cells, the average viability at each concentration was plotted as bar graph and the positive control wells is presented as a dotted line.

FIG. 5: Graphs of dose responses in A375 and U87 Cells. Percent Viability was normalized wherein 0% was defined as smallest mean and 100% was defined as largest mean in each data set. It was then plotted as a function of the construct concentration (pM). The average viability of the positive control wells is presented as a dotted line for bar graphs.

FIG. 6: Graphs of dose responses in EMT6-IL13Rα2 and EMT6 wild type Cells. Percent Viability was normalized wherein 0% was defined as smallest mean and 100% was defined as largest mean in each data set for EMT6-IL13Rα2. It was then plotted as a function of the construct concentration (pM). For EMT6 wild type cells, the average viability at each concentration was plotted as bar graph and the positive control wells is presented as a dotted line.

FIG. 7: Graphs showing IL-13 superkine MDNA213 (also referred to as MDNA132.15, SEQ ID NO:106) fused to Pseudomonas aeruginosa exotoxin (PE) induces tumor growth inhibition in IL-13Rα2 expressing cell lines. MDNA213-PE exhibits tumor growth inhibition in EMT6-IL13Rα2 tumors specifically (bottom) with an insignificant response in EMT-6 wild type tumors (top). The MDNA213-PE response was observed to synergize in combination with IL-2 super-agonist, MDNA19 to significantly enhance the therapeutic efficacy.

### DETAILED DESCRIPTION

In order for the present disclosure to be more readily understood, certain terms and phrases are defined below as well as throughout the specification.

### Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, NY 2001); March, Advanced Organic Chemistry Reactions, Mechanisms and Structure 5th ed., J. Wiley & Sons (New York, NY 2001); and Sambrook and Russell, Molecular Cloning: A Laboratory Manual 3rd ed., Cold Spring harbor Laboratory Press (Cold Spring Harbor, NY 2001), provide one skilled in the art with a general guide to many terms used in the present disclosure. As appropriate, procedures involving the use of commercially available kits and reagents are generally carried out in accordance with manufacturer defined protocols and/or parameters unless otherwise noted.

As used herein, “IL-2” means wild-type IL-2, whether native or recombinant. Mature human IL-2 occurs as a 133 amino acid sequence (less the signal peptide, consisting of an additional 20 N-terminal amino acids), as described in Fujita, et. al., PNAS USA, 80, 7437-7441 (1983). The amino acid sequence of human IL-2 (SEQ ID NO:301; full length) is found in Genbank under accession locator NP_000577.2. The amino acid sequence of mature human IL-2 is depicted in SEQ ID NO:302 (human wild-type mature; position numbering of the substitutions is based on this sequence). The murine (Mus musculus) IL-2 amino acid sequence is found in Genbank under accession locator (SEQ ID NO:303). The amino acid sequence of mature murine IL-2 is depicted in SEQ ID NO:304.

SEQ ID NO: 301
MYRMQLLSCIALSLALVTNSAPTSSSTKKTQLQLEHLLLDLQMIL
NGINNYKNPKLTRMLTFKFYMPKKATELKHLQCLEEELKPLEEVL
NLAQSKNFHLRPRDLISNINVIVLELKGSETTFMCEYADETATIV
EFLNRWITFCQSIISTLT
SEQ ID NO: 302
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFCQSIISTLT
SEQ ID NO: 303
MYSMQLASCVTLTLVLLVNSAPTSSSTSSSTAEAQQQQQQQQQQQ
QHLEQLLMDLQELLSRMENYRNLKLPRMLTFKFYLPKQATELKDL
QCLEDELGPLRHVLDLTQSKSFQLEDAENFISNIRVTVVKLKGSD
NTFECQFDDESATVVDFLRRWIAFCQSIISTSPQ
SEQ ID NO: 304
APTSSSTSSSTAEAQQQQQQQQQQQQHLEQLLMDLQELLSRMENY
RNLKLPRMLTFKFYLPKQATELKDLQCLEDELGPLRHVLDLTQSK
SFQLEDAENFISNIRVTVVKLKGSDNTFECQFDDESATVVDFLRR
WIAFCQSIISTSPQ

As used herein, “IL-2 mutein” means an IL-2 polypeptide wherein specific substitutions to the interleukin-2 protein have been made. The IL-2 muteins are characterized by amino acid insertions, deletions, substitutions and modifications at one or more sites in or at the other residues of the native IL-2 polypeptide chain. In accordance with this disclosure, any such insertions, deletions, substitutions and modifications result in an IL-2 mutein that retains the IL-2Rβ binding activity. Exemplary muteins can include substitutions of 1, 2, 3, 4, 5, 6, 7, 8, 9, 10 or more amino acids.

Muteins also include conservative modifications and substitutions at other positions of IL-2 (i.e., those that have a minimal effect on the secondary or tertiary structure of the mutein). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: ala, pro, gly, gln, asn, ser, thr; Group II: cys, ser, tyr, thr; Group III:val, ile, leu, met, ala, phe; Group IV: lys, arg, his; Group V: phe, tyr, trp, his; and Group VI: asp, glu.

“Numbered in accordance with IL-2” means identifying a chosen amino acid with reference to the position at which that amino acid normally occurs in the mature sequence of wild type IL-2, for example R81 refers to the eighty-first amino acid, arginine, that occurs in SEQ ID NO:302. L80 refers to the eightieth amino acid, leucine, that occurs in SEQ ID NO:302. L85 refers to the eighty-fifth amino acid, leucine, that occurs in SEQ ID NO:2. 186 refers to the eighty-sixth amino acid, isoleucine, that occurs in SEQ ID NO:302. 192 refers to the ninety-second amino acid, isoleucine, that occurs in SEQ ID NO:302. F42 refers to the forty-second amino acid, phenylalanine, that occurs in SEQ ID NO:302. K43 refers to the forty-third amino acid, lysine, that occurs in SEQ ID NO:302.

As used herein, the abbreviations for the genetically encoded L-enantiomeric amino acids used in the disclosure methods are conventional and are as follows in Table 1.

TABLE 1
Amino acid abbreviations

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

“Hydrophilic Amino Acid” refers to an amino acid exhibiting a hydrophobicity of less than zero according to the normalized consensus hydrophobicity scale of Eisenberg et aL., 1984, J. Mol. Biol. 179: 125-142. Genetically encoded hydrophilic amino acids include Thr (T), Ser (S), His (H), Glu (E), Asn (N), Gln (Q), Asp (D), Lys (K) and Arg (R).

The term “cell types having the IL-2αβγ receptor” means the cells known to have this receptor type, i.e., T cells, activated T cells, B cells, activated monocytes, and activated NK cells. The term “cell types having the IL-2Rβγ receptor” means the cells known to have that receptor type, i.e., B cells, resting monocytes, and resting NK cells.

The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The terms “polypeptide,” “protein” or “peptide” refer to any chain of amino acid residues, regardless of its length or post-translational modification (e.g., glycosylation or phosphorylation).

In the event the mutant IL-2 polypeptides of the disclosure are “substantially pure,” they can be at least about 60% by weight (dry weight) the polypeptide of interest, for example, a polypeptide containing the mutant IL-2 amino acid sequence. For example, the polypeptide can be at least about 75%, about 80%, about 85%, about 90%, about 95% or about 99%, by weight, the polypeptide of interest. Purity can be measured by any appropriate standard method, for example, column chromatography, polyacrylamide gel electrophoresis, or HPLC analysis.

An “agonist” is a compound that interacts with a target to cause or promote an increase in the activation of the target.

A “partial agonist” is a compound that interacts with the same target as an agonist but does not produce as great a magnitude of a biochemical and/or physiological effect as the agonist, even by increasing the dosage of the partial agonist.

A “superagonist” (also referred to as a “superkine”) is a type of agonist that is capable of producing a maximal response greater than the endogenous agonist for the target receptor, and thus has an efficacy of more than 100%.

“Operably linked” is intended to mean that the nucleotide sequence of interest (i.e., a sequence encoding an IL-2 mutein) is linked to the regulatory sequence(s) in a manner that allows for expression of the nucleotide sequence (e.g., in an in vitro transcription/translation system or in a host cell when the vector is introduced into the host cell). “Regulatory sequences” include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression constructs of the invention can be introduced into host cells to thereby produce the human IL-2 muteins disclosed herein or to produce biologically active variants thereof.

The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell but are still included within the scope of the term as used herein.

As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, particle gun, or electroporation.

As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics) can also be incorporated into the compositions.

As used herein, the term “anti-PD-1 antibody” refers to any antibody that binds to PD-1, including inhibitory antibodies. An “anti-PD-1 inhibitor” refers to an inhibitor that binds to and inhibits PD-1. Such anti-PD-1 antibodies and/or inhibitors include but are not limited to nivolumab, BMS-936558, MDX-1106, ONO-4538, AMP224, CT-011, and MK-3475, among others.

As used herein, the terms “cancer” (or “cancerous”), “hyperproliferative,” “tumor” and/or “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, reproductive systems, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus. Cancers generally can include solid tumors, as well as sarcoma, carcinoma, head and neck cancer, glioblastoma, bladder cancer, oral cancer, mesothelioma, pancreatic cancer, liver cancer, colorectal cancer, pulmonary cancer, cutaneous, lymphoid, gastrointestinal cancer, prostate cancer, ovarian cancer, breast cancer, basal-like breast tumor, endometrial cancer, multiple myeloma, melanoma, lymphoma, lung cancer (including small cell lung cancer), kidney cancer, gastric cancer, brain cancer, and CNS tumors. CNS tumors include glioma, glioblastoma, glioblastoma multiforme (GBM), refractory glioblastoma multiforme (rGBM), recurrent glioblastoma, astrocytoma, medulloblastoma, craniopharyogioma, ependymoma, pinealoma, hemangioblastoma, acoustic neuroma, oligodendroglia, menangioma, meningioma, neuroblastoma, retinoblastoma, medulloblastoma, adult pituitary adenoma, an 06-methylguanine-methyltransferase (MGMT) positive or negative CNS tumor, and furin positive CNS tumor.

The term “carcinoma” is art-recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

As used herein, the term “hematopoietic neoplastic disorders” refers to diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol/Hemotol. 11:267-97); lymphoid malignancies include but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Stemberg disease.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease. A therapeutically effective amount can be an amount that reduces tumor number, tumor size, and/or increases survival.

The terms “individual,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

The terms “pharmaceutically acceptable” and “physiologically acceptable” mean a biologically acceptable formulation, gaseous, liquid or solid, or mixture thereof, suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering an IL-2 mutein to a subject. In particular, an IL-2 mutein comprising the substitutions L80F, R81D, L85V, 186V, and 192F is administered in combination with anti-PD-1 to a subject with cancer. In some embodiments, the IL-2 mutein administered further comprises a substitution at position F42A. In some embodiments, the IL-2 administered mutein further comprises a substitution at position K43N.

The phrase a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, produces a desired effect (e.g., prophylactic or therapeutic effect). In some embodiments, the therapeutic effect is to reduce tumor number. In some embodiments, the therapeutic effect is to reduce tumor size. In some embodiments, the therapeutic effect is to increase survival.

In some embodiments, unit dosage forms may be within, for example, ampules and vials, including a liquid composition, or a composition in a freeze-dried or lyophilized state; a sterile liquid carrier, for example, can be added prior to administration or delivery in vivo. Individual unit dosage forms can be included in multi-dose kits or containers. IL-2 muteins in combination with anti-PD-1 antibodies, and pharmaceutical compositions thereof can be packaged in a single or multiple unit dosage form for ease of administration and uniformity of dosage.

A “therapeutically effective amount” will fall in a relatively broad range determinable through experimentation and/or clinical trials. For example, for in vivo injection, e.g., injection directly into the tissue or vasculature of a subject (for example, liver tissue or veins). Other effective dosages can be readily established by one of ordinary skill in the art through routine trials establishing dose response curves.

An “effective amount” or “sufficient amount” refers to an amount providing, in single or multiple doses, alone or in combination, with one or more other compositions (therapeutic agents such as a drug), treatments, protocols, or therapeutic regimens agents (including, for example, vaccine regimens), a detectable response of any duration of time (long or short term), an expected or desired outcome in or a benefit to a subject of any measurable or detectable degree or for any duration of time (e.g., for minutes, hours, days, months, years, or cured).

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is also a satisfactory outcome. In some embodiments, the effective amount is an amount sufficient to reduce tumor number. In some embodiments, the effective amount is an amount sufficient to reduce tumor size. In some embodiments, the effective amount is an amount sufficient to increase survival.

“Prophylaxis” and grammatical variations thereof mean a method in which contact, administration or in vivo delivery to a subject is prior to disease. Administration or in vivo delivery to a subject can be performed prior to development of an adverse symptom, condition, complication, etc. caused by or associated with the disease. For example, a screen (e.g., genetic) can be used to identify such subjects as candidates for the described methods and uses, but the subject may not manifest the disease. Such subjects therefore include those screened positive for an insufficient amount or a deficiency in a functional gene product (protein), or producing an aberrant, partially functional or non-functional gene product (protein), leading to disease; and subjects screening positive for an aberrant, or defective (mutant) gene product (protein) leading to disease, even though such subjects do not manifest symptoms of the disease.

### I. DETAILED DESCRIPTION

Described herein are methods of treating a cancer comprising administering: a) an IL-4 receptor targeted cargo protein or an IL-13 receptor targeted cargo protein; and b) immunomodulatory agent (e.g., an IL-2 mutein described herein). In some embodiments, the IL-4 receptor targeted cargo protein includes an IL-4, an IL13, an IL-4 mutein, or an IL-13 mutein linked to a cargo moiety. In some embodiments, the IL-13 receptor targeted cargo protein includes an IL-4, or an IL-13 (e.g., any of the IL-4 muteins or IL-13 muteins or fusion proteins disclosed herein) linked to a cargo moiety. In some embodiments, the cargo moiety functions to reduce or inhibit cancer stem cell growth, or kill cancer cells and/or cancer stem cells. In some embodiments, the immunomodulatory agent is an IL-2 mutein disclosed herein. As disclosed below, the tumor growth inhibitory activity of IL-4s or IL-13s targeted cargo moieties and IL-2 are advantageously enhanced when administered in combination as compared to individually (see Example 6). Such combination therapies are particularly useful for the treatment of cancers that overexpress IL-4R or IL-13Rα2. Also provided herein are composition comprising: a) an IL-4 receptor targeted cargo protein or an IL-13 receptor targeted cargo protein; and b) an IL-2 mutein. Aspects of the methods and compositions are further detailed below.

### A. IL-2 Muteins

The subject cancer treatment methods include a step of administering: a) an IL-4 receptor targeted cargo protein or an IL-13 receptor targeted cargo protein; and b) immunomodulatory agent. In some embodiments, the immunomodulatory agent comprises an IL-2 mutein, wherein the IL-2 mutein includes one or more amino acid substitutions are compared to human IL-2.

The substituted amino acid residue(s) can be, but are not necessarily, conservative substitutions, which typically include substitutions within the following groups: glycine, alanine; valine, isoleucine, leucine; aspartic acid, glutamic acid; asparagine, glutamine; serine, threonine; lysine, arginine; and phenylalanine, tyrosine. These mutations can be at amino acid residues that contact the IL-2Rβ and/or the IL-2Rγ.

More specifically, a mutation (whether conservative or non-conservative, by way of addition(s) or deletion(s)) can be made at one or more of positions. For example, the mutation can be: 124V, P65H, Q74R, Q74H, Q74N, Q74S, L80F, L80V, R811, R81T, R81D, L85V, 186V, 189V, 192F, V931. The sequences of exemplary IL-2 muteins are provided in SEQ ID NOs:1-20.

In some embodiments, the substitutions in the IL-2 mutein comprise L80F, R81D, L85V, 186V, and 192F. In some embodiments, the IL-2 mutein further comprises F42A substitution. In some embodiments, the IL-2 mutein further comprises Y45A substitution. In some embodiments, the IL-2 mutein further comprises E62A substitution. In some embodiments, the substitutions in the IL-2 mutein comprise F42A, L80F, R81D, L85V, 186V, and 192F. In some embodiments, the substitutions in the IL-2 mutein comprise F42A, Y45A, L80F, R81D, L85V, 186V, and 192F. In some embodiments, the substitutions in the IL-2 mutein comprise F42A, E62A, L80F, R81D, L85V, 186V, and 192F. In some embodiments, the substitutions in the IL-2 mutein comprise F42A, Y45A, E62A, L80F, R81D, L85V, 186V, and 192F. In some embodiments, the substitutions in the IL-2 mutein comprise E62A, L80F, R81D, L85V, 186V, and 192F. In some embodiments, the substitutions in the IL-2 mutein comprise Y45A, E62A, L80F, R81D, L85V, 186V, and 192F. In some embodiments, the substitutions in the IL-2 mutein comprise Y45A and E62A.

In some embodiments, the substitutions in the IL-2 mutein that lead to increased and/or enhanced IL-2Rβ binding include L80F, R81D, L85V, 186V, and 192F. In some embodiments, an IL-2 mutein for use in the invention comprises L80F, R81D, L85V, 186V, and 192F and exhibits increased IL-2Rβ binding. In some embodiments, an IL-2 mutein for use in the invention further comprises a substitution at position F42A. In some embodiments, the IL-2 mutein for use in the invention further comprises a substitution at position K43N. In some embodiments, the mutein comprises substitutions L80F, R81D, L85V, 186V, and 192F, and one or more substitutions selected from the group consisting of F42A, Y45A, and E62A, all as compared to wild-type human IL-2.

In some embodiments, the amino acid substitutions increasing IL-2Rβ binding affinity include: L80F, R81D, L85V, 186V, and 192F. In some embodiments, the amino acid substitutions that increase IL-2Rβbinding affinity include: L80F, R81D, L85V, 186V, and 192F.

In some embodiments, the subject IL-2 mutein having a greater binding affinity for IL-2Rβ as compared to wild-type human IL-2, includes the amino acid substitutions L80F, R81D, L85V, 186V, and 192F. In some embodiments, the IL-2 mutein has the amino acid sequence:

(SEQ ID NO: 1; H9 as used in the Examples)
APTSSSTKKTQLQLEHLLLDLQMILNGINNYKNPKLTRMLTFKFY
MPKKATELKHLQCLEEELKPLEEVLNLAQSKNFHFDPRDVVSNIN
VEVLELKGSETTFMCEYADETATIVEFLNRWITFCQSIISTLT.

In some embodiments, the IL-2 mutein has increased capabilities to stimulate one or more signaling pathways that are dependent on IL-2Rβ/IL-2Rγ_{c} heterodimerization. In some embodiments, the subject IL-2 mutein has an enhanced capability to stimulate STAT5 phosphorylation in an IL-2Rβ+ cell as compared to wild-type human IL-2. In some embodiments, the IL-2 mutein stimulates STAT5 phosphorylation in an IL-2Rβ+ cell at a level that is 1%, 5%, 10%, 15%, 20%, 25%, 30%, 35%, 40%, 45%, 50%, 55%, 60%, 65%, 70%, 75%, 80%, 85%, 90%, 95% or more of the level that wild-type IL-2 stimulates STAT5 phosphorylation in the same cell. In some embodiments, the IL-2 mutein stimulates STAT5 phosphorylation in an IL-2Rβ+ cell at a level that is 105%, 110%, 115%, 120%, 125%, 130%, 135%, 140%, 145%, 150%, 155%, 160%, 165%, 170%, 175%, 180%, 185%, 190%, 195% or more as compared to the level that wild-type IL-2 stimulates STAT5 phosphorylation in the same cell. In some embodiments, the IL-2Rβ+ cell is a T cell. In particular embodiments, the T cell is a CD8+ T cell. In some embodiments, the CD8+ T cell is a freshly isolated CD8+ T cell. In other embodiments, the CD8+ T cell T cell is an activated CD8+ T cell. In other embodiments, the IL-2Rβ+ cell is a natural killer (NK) cell. In some embodiments, the IL-2 mutein comprises substitutions L80F, R81D, L85V, 186V, and 192F, as compared to wild-type human IL-2.

In some embodiments, the mutein has an enhanced capability to stimulate ERK1/ERK2 signaling in an IL-2Rβ+ cell as compared to wild-type human IL-2. In some embodiments, the IL-2 mutein stimulates pERK1/ERK2 signaling in an IL-2Rβ+ cell at a level that is 1%, 5%, 10%, 15%, 20%, 25%, 30%, 35%, 40%, 45%, 50%, 55%, 60%, 65%, 70%, 75%, 80%, 85%, 90%, 95% or more of the level that wild-type IL-2 stimulates pERK1/ERK2 signaling in the same cell. In some embodiments, the IL-2 mutein stimulates pERK1/ERK2 phosphorylation in an IL-2Rβ+ cell at a level that is 105%, 110%, 115%, 120%, 125%, 130%, 135%, 140%, 145%, 150%, 155%, 160%, 165%, 170%, 175%, 180%, 185%, 190%, 195% or more as compared to the level that wild-type IL-2 stimulates pERK1/ERK2 phosphorylation in the same cell. In some embodiments, the IL-2Rβ+ cell is a T cell. In particular embodiments, the T cell is a CD8+ T cell. In some embodiments, the CD8+ T cell is a freshly isolated CD8+ T cell. In other embodiments, the CD8+ T cell T cell is an activated CD8+ T cell. In other embodiments, the IL-2Rβ+ cell is a natural killer (NK) cell. In some embodiments, the IL-2 mutein comprises substitutions L80F, R81D, L85V, 186V, and 192F, as compared to wild-type human IL-2.

STAT5 and ERK1/2 signaling can be measured, for example, by phosphorylation of STAT5 and ERK1/2 using any suitable method known in the art. For example, STAT5 and ERK1/2 phosphorylation can be measured using antibodies specific for the phosphorylated version of these molecules in combination with flow cytometry analysis as described herein. In some embodiments, the mutein has an enhanced capability to stimulate PI 3-kinase signaling in a IL-2Rβ+ cell as compared to wild-type human IL-2. In some embodiments, the IL-2 mutein stimulates PI 3-kinase signaling in an IL-2Rβ+ cell at a level that is 1%, 5%, 10%, 15%, 20%, 25%, 30%, 35%, 40%, 45%, 50%, 55%, 60%, 65%, 70%, 75%, 80%, 85%, 90%, 95% or less of the level that wild-type IL-2 stimulates PI 3-kinase signaling in the same cell. In some embodiments, the IL-2 mutein stimulates PI 3-kinase signaling in an IL-2Rβ+ cell at a level that is 105%, 110%, 115%, 120%, 125%, 130%, 135%, 140%, 145%, 150%, 155%, 160%, 165%, 170%, 175%, 180%, 185%, 190%, 195% or more as compared to the level that wild-type IL-2 stimulates PI 3-kinase signaling phosphorylation in the same cell. In some embodiments, the IL-2Rβ+ cell is a T cell. In particular embodiments, the T cell is a CD8+ T cell. In some embodiments, the CD8+ T cell T cell is an activated CD8+ T cell. In other embodiments, the IL-2Rβ+ cell is a natural killer (NK) cell. In some embodiments, the IL-2 mutein comprises substitutions L80F, R81D, L85V, 186V, and 192F, as compared to wild-type human IL-2. P13-kinase signaling can be measured using any suitable method known in the art. For example, PI 3-kinase signaling can be measured using antibodies that are specific for phospho-S6 ribosomal protein in conjunction with flow cytometry analysis as described herein.

In some embodiments the IL-2 mutein is a stimulator of IL-2 and/or IL-15 STAT5 phosphorylation in CD8+ T cells. In some embodiments, the mutein is a promoter of IL-2 and/or IL-15 induced proliferation of CD8+ T cells. In some embodiments, the mutein is a stimulator of IL-2 dependent, TCR-induced cell proliferation. In some embodiments, the IL-2 mutein comprises substitutions L80F, R81D, L85V, 186V, and 192F, as compared to wild-type human IL-2.

IL-2 promotes Th1, Th9, and Treg T cell differentiation and inhibits Th17 differentiation. Therefore, without being bound by any particular theory of operation, it is believed that IL-2 muteins that function as IL-2 superagonists are capable of promoting Th1, Th9, and/or Treg cell differentiation or inhibiting Th17 cell differentiation. In some embodiments, the IL-2 mutein is a promoter of IL-2 dependent Th1, Th9 and/or Treg differentiation. In some embodiments, the mutein is an inhibitor of Th17 differentiation. In some embodiments, the IL-2 mutein comprises substitutions L80F, R81D, L85V, 186V, and 192F, as compared to wild-type human IL-2.

In some embodiments, the IL-2 mutein signals less and/or independently of CD25 (for example, has reduced or ablated CD25 binding) as compared to wild-type human IL-2. In some embodiments the reduced and/or independent signaling with regard to CD25 allows for preferential activation of effector T-cells while limiting the stimulation of Tregs. In some embodiments the reduced and/or independent signaling with regard to CD25 allows for reduced toxicity. In some embodiments, the mutein comprises substitutions L80F, R81D, L85V, 186V, and 192F, and one or more substitutions selected from the group consisting of F42A, Y45A, and E62A, all as compared to wild-type human IL-2.

In some embodiments, the IL-2 mutein is capable of increasing and/or restoring responsiveness to anergic NK cells. In some embodiments, the IL-2 mutein is capable of increasing and/or restoring responsiveness to anergic NK cells in the tumor microenvironment. In some embodiments, the IL-2 mutein comprises substitutions L80F, R81D, L85V, 186V, and 192F, as compared to wild-type human IL-2.

In some embodiments the mutein is an inhibitor an inhibitor of IL-2 dependent activation of natural killer (NK) cells. IL-2 activation of NK cells can be measured by any suitable method known in the art, for example, by measuring IL-2 induced CD69 expression and/or cytotoxicity, as described herein.

In some embodiments, an increase in IL-2Rβ binding affinity is any binding affinity for IL-2Rβ that is greater than the wild-type human IL-2 binding affinity for IL-2Rβ. In some embodiments, the binding affinity is a 2-fold, 5-fold, 10-fold, 20-fold, 30-fold, 40-fold, 50-fold, 60-fold, 70-fold, 80-fold, 90-fold, 100-fold, 120-fold, 150-fold, 170-fold, 190-fold, 200-fold, 220-fold, 240-fold or more increase in binding affinity for IL-2Rβ as compared to the wild-type human IL-2 binding affinity for IL-2Rβ.

In some embodiments, an increase in binding capacity for IL-2Rβ is any binding capacity for IL-2R3 that is greater than the wild-type human IL-2 binding capacity for IL-2Rβ. In some embodiments, the binding capacity is a 2-fold, 5-fold, 10-fold, 20-fold, 30-fold, 40-fold, 50-fold, 60-fold, 70-fold, 80-fold, 90-fold, 100-fold, 120-fold, 150-fold, 170-fold, 190-fold, 200-fold, 220-fold, 240-fold or more increase in binding capacity for IL-2Rβ as compared to the wild-type human IL-2 binding capacity for IL-2Rβ.

In some embodiments, the subject IL-2 mutein having a greater binding affinity for IL-2Rβ as compared to wild-type human IL-2 also exhibits reduced binding to CD25 and includes the amino acid substitutions F42A, L80F, R81D, L85V, 186V, and 192F. In some embodiments, the reduce binding affinity is about 220-fold, i.e., from about Kd of 6.6 nM for wild-type human IL-2 to about 1.4 μM for the mutein comprising F42A, L80F, R81D, L85V, 186V, and 192F. In some embodiments, the IL-2 mutein has the amino acid sequence:

(SEQ ID NO: 2; also referred to as H9-F42A)
APTSSSTKKTQLQLEHLLLDLQMILNGINNYKNPKLTRMLTAKFY
MPKKATELKHLQCLEEELKPLEEVLNLAQSKNFHFDPRDVVSNIN
VEVLELKGSETTFMCEYADETATIVEFLNRWITFCQSIISTLT.

In some embodiments, the subject IL-2 mutein having a greater binding affinity for IL-2Rβ as compared to wild-type human IL-2 also exhibits reduced binding to CD25 and includes the amino acid substitutions K43N, L80F, R81D, L85V, 186V, and 192F. In some embodiments, the reduce binding affinity is due to allowing for glycosylation at position 43 with the K43N substitution. By substituting lysine for asparagine (K43N), CD25 binding is reduced and/or ablated in the IL-2 mutein comprising the amino acid substitutions K43N, L80F, R81D, L85V, 186V, and 192F. In some embodiments, the IL-2 mutein has the amino acid sequence:

(SEQ ID NO: 3; also referred to as H9-K43N)
APTSSSTKKTQLQLEHLLLDLQMILNGINNYKNPKLTRMLTFNFY
MPKKATELKHLQCLEEELKPLEEVLNLAQSKNFHFDPRDVVSNIN
VEVLELKGSETTFMCEYADETATIVEFLNRWITFCQSIISTLT.

In some embodiments, a reduction in binding affinity for CD25 is any binding affinity for CD25 that is less than the wild-type human IL-2 binding affinity. In some embodiments, the binding affinity is a 10-fold, 20-fold, 30-fold, 40-fold, 50-fold, 60-fold, 70-fold, 80-fold, 90-fold, 100-fold, 120-fold, 150-fold, 170-fold, 190-fold, 200-fold, 220-fold, 240-fold or more decrease in binding affinity for CD25 as compared to the wild-type human IL-2 binding affinity for CD25.

In some embodiments, the subject IL-2 mutein having a greater binding affinity for IL-2Rβ and a reduced binding affinity for CD25 as compared to wild-type human IL-2 includes the amino acid substitutions F42A, Y45A L80F, R81D, L85V, 186V, and 192F. In some embodiments, the IL-2 mutein has the amino acid sequence:

(SEQ ID NO: 4; H9-F42A/Y45A; H9-FYAA)
APTSSSTKKTQLQLEHLLLDLQMILNGINNYKNPKLTRMLTAKFA
MPKKATELKHLQCLEEELKPLEEVLNLAQSKNFHFDPRDVVSNIN
VEVLELKGSETTFMCEYADETATIVEFLNRWITFCQSIISTLT.

In some embodiments, the subject IL-2 mutein having a greater binding affinity for IL-2Rβ and a reduced binding affinity for CD25 as compared to wild-type human IL-2 includes the amino acid substitutions F42A, E62A L80F, R81D, L85V, 186V, and 192F. In some embodiments, the IL-2 mutein has the amino acid sequence:

(SEQ ID NO: 5; H9-F42A/E62A; H9-FEAA)
APTSSSTKKTQLQLEHLLLDLQMILNGINNYKNPKLTRMLTAKFY
MPKKATELKHLQCLEEALKPLEEVLNLAQSKNFHFDPRDVVSNIN
VEVLELKGSETTFMCEYADETATIVEFLNRWITFCQSIISTLT.

In some embodiments, the subject IL-2 mutein having a greater binding affinity for IL-2Rβ and a reduced binding affinity for CD25 as compared to wild-type human IL-2 includes the amino acid substitutions F42A, Y45A, E62A, L80F, R81D, L85V, 186V, and 192F. In some embodiments, the IL-2 mutein has the amino acid sequence:

(SEQ ID NO: 6; H9-F42A/Y45A/E62A; H9-FYEAAA)
APTSSSTKKTQLQLEHLLLDLQMILNGINNYKNPKLTRMLTAKFA
MPKKATELKHLQCLEEALKPLEEVLNLAQSKNFHFDPRDVVSNIN
VEVLELKGSETTFMCEYADETATIVEFLNRWITFCQSIISTLT.

In some embodiments, the IL-2 mutein sequence is 90% identical to any one of the wild type or SEQ ID NO:1 through SEQ ID NO:20. In some embodiments, the IL-2 mutein sequence is 95% identical to any one of the wild type or SEQ ID NO:1 through SEQ ID NO:20. In some embodiments, the IL-2 mutein sequence is 98% identical to any one of the wild type or SEQ ID NO:1 through SEQ ID NO:20. In some embodiments, the IL-2 mutein sequence is 99% identical to any one of the wild type or SEQ ID NO:1 through SEQ ID NO:20.

Exemplary IL-2 muteins that are useful as an immunomodulatory agent in the subject methods are provided in the table below.

TABLE 2
Exemplary IL-2 Muteins

| SEQ ID NO | |
|---|---|
| (Information) | Amino acid sequence |
| SEQ ID NO: 1 | APTSSSTKKTQLQLEHLLLDLQMILNGINNYKNPKLTRMLTFKFYMPKKATELKHLQC |
| (H9) | LEEELKPLEEVLNLAQSKNFHFDPRDVVSNINVFVLELKGSETTFMCEYADETATIVE |
| | FLNRWITFCQSIISTLT |
| SEQ ID NO: 2 | APTSSSTKKTQLQLEHLLLDLQMILNGINNYKNPKLTRMLTAKFYMPKKATELKHLQC |
| (H9-F42A) | LEEELKPLEEVLNLAQSKNFHFDPRDVVSNINVFVLELKGSETTFMCEYADETATIVE |
| | FLNRWITFCQSIISTLT |
| SEQ ID NO: 3 | APTSSSTKKTQLQLEHLLLDLQMILNGINNYKNPKLTRMLTFNFYMPKKATELKHLQC |
| (H9-K43N) | LEEELKPLEEVLNLAQSKNFHFDPRDVVSNINVFVLELKGSETTFMCEYADETATIVE |
| | FLNRWITFCQSIISTLT |
| SEQ ID NO: 4 | APTSSSTKKTQLQLEHLLLDLQMILNGINNYKNPKLTRMLTAKFAMPKKATELKHLQC |
| (A2) (H9-F42A, | LEEELKPLEEVLNLAQSKNFHFDPRDVVSNINVFVLELKGSETTFMCEYADETATIVE |
| Y45A) (H9-FYAA) | FLNRWITFCQSIISTLT |
| SEQ ID NO: 5 | APTSSSTKKTQLQLEHLLLDLQMILNGINNYKNPKLTRMLTAKFYMPKKATELKHLQC |
| (B1) (H9-F42A, | LEEALKPLEEVLNLAQSKNFHFDPRDVVSNINVFVLELKGSETTFMCEYADETATIVE |
| E62A) (H9-FEAA) | FLNRWITFCQSIISTLT |
| SEQ ID NO: 6 | APTSSSTKKTQLQLEHLLLDLQMILNGINNYKNPKLTRMLTAKFAMPKKATELKHLQC |
| (B11) (F42A, Y45A, | LEEALKPLEEVLNLAQSKNFHFDPRDVVSNINVFVLELKGSETTFMCEYADETATIVE |
| E62A) (H9-FYEAAA) | FLNRWITFCQSIISTLT |
| SEQ ID NO: 7 | APTSSSTKKTQLQLEHLLLDLQMILNGINNYKNPKLTRMLTFKFYMPKKATELKHLQC |
| (Q74R) | LEEELKPLEEVLNLARSKNFHLRPRDLISNINVIVLELKGSETTFMCEYADETATIVE |
| | FLNRWITFCQSIISTLT |
| SEQ ID NO: 8 | APTSSSTKKTQLQLEHLLLDLQMILNGINNYKNPKLTRMLTFKFYMPKKATELKHLQC |
| (Q74R, L85V) | LEEELKPLEEVLNLARSKNFHLRPRDVISNINVIVLELKGSETTFMCEYADETATIVE |
| | FLNRWITFCQSIISTLT |
| SEQ ID NO: 9 | APTSSSTKKTQLQLEHLLLDLQMILNGINNYKNPKLTRMLTFKFYMPKKATELKHLQC |
| (Q74R, R81I, L85V) | LEEELKPLEEVLNLARSKNFHLIPRDVISNINVIVLELKGSETTFMCEYADETATIVE |
| | FLNRWITFCQSIISTLT |
| SEQ ID NO: 10 | APTSSSTKKTQLQLEHLLLDLQMILNGINNYKNPKLTRMLTFKFYMPKKATELKHLQC |
| (Q74H, R81T, L85V, | LEEELKPLEEVLNLAHSKNFHLTPRDVVSNINVFILELKGSETTFMCEYADETATIVE |
| I86V, I92F, V93I) | FLNRWITFCQSIISTLT |
| SEQ ID NO: 11 | APTSSSTKKTQLQLEHLLLDLQMILNGINNYKNPKLTRMLTFKFYMPKKATELKHLQC |
| (Q74N, L80F, R81D, | LEEELKPLEEVLNLANSKNFHFDPRDVVSNVNVFVLELKGSETTFMCEYADETATIVE |
| L85V, 186V, I89V, | FLNRWITFCQSIISTLT |
| I92F) | |
| SEQ ID NO: 12 | APTSSSTKKTQLQLEHLLLDLQMVLNGINNYKNPKLTRMLTFKFYMPKKATELKHLQC |
| (I24V, Q74S, L80F, | LEEELKPLEEVLNLASSKNFHFDPRDVVSNINVFVLELKGSETTFMCEYADETATIVE |
| R81D, L85V, 186V, | FLNRWITFCQSIISTLT |
| I92F) | |
| SEQ ID NO: 13 | APTSSSTKKTQLQLEHLLLDLQMILNGINNYKNPKLTRMLTFKFYMPKKATELKHLQC |
| (P65H, Q74N, L80V, | LEEELKHLEEVLNLANSKNFHVTPRDVVSNINVFVLELKGSETTFMCEYADETATIVE |
| R81T, L85V, I86V, | FLNRWITFCQSIISTLT |
| I92F) | |
| SEQ ID NO: 14 | APTSSSTKKTQLQLEHLLLDLQMILNGINNYKNPKLTRMLTFKFYMPKKATELKHLQC |
| (Q74H, L80F, R81D, | LEEELKPLEEVLNLAHSKNFHFDPRDVVSNINVFVLELKGSETTFMCEYADETATIVE |
| L85V, I86V, 192F) | FLNRWITFCQSIISTLT |
| (H9-Q74H) (H9D10) | |
| SEQ ID NO: 15 | APTSSSTKKTQLQLEHLLLDLQMILNGINNYKNPKLTRMLTFKFYMPKKATELKHLQC |
| (Q74S) (H9E10) | LEEELKPLEEVLNLASSKNFHFDPRDVVSNINVFVLELKGSETTFMCEYADETATIVE |
| | FLNRWITFCQSIISTLT |
| SEQ ID NO: 16 | APTSSSTKKTQLQLEHLLLDLQMILNGINNYKNPKLTRMLTFKFYMPKKATELKHLQC |
| (Q74N) (H9G8) | LEEELKPLEEVLNLANSKNFHFDPRDVVSNINVFVLELKGSETTFMCEYADETATIVE |
| | FLNRWITFCQSIISTLT |
| SEQ ID NO: 17 | APTSSSTKKTQLQLEHLLLDLQMILNGINNYKNPKLTRMLTFKFYMPKKATELKHLQC |
| (Q74S, R81T, L85V, | LEEELKPLEEVLNLASSKNFHLTPRDVISNINVFVLELKGSETTFMCEYADETATIVE |
| I92F) | FLNRWITFCQSIISTLT |
| SEQ ID NO: 18 | APTSSSTKKTQLQLEHLLLDLQMILNGINNYKNPKLTRMLTFKFYMPKKATELKHLQC |
| (H9-Q74N, I89V) | LEEELKPLEEVLNLANSKNFHFDPRDVVSNVNVFVLELKGSETTFMCEYADETATIVE |
| (H9B1) | FLNRWITFCQSIISTLT |
| SEQ ID NO: 19 | APTSSSTKKTQLQLEHLLLDLQMILNGINNYKNPKLTRMLTAKFYMPKKATELKHLQC |
| (F42A, E62A, L80F, | LEEALKPLEEVLNLAQSKNFHFDPRDVVSNINVFVLELKGSETTFMCEYADETATIVE |
| R81D, L85V, I86V, | FLNRWITFSQSIISTLT |
| I92F, C125S) | |
| SEQ ID NO: 20 | APTSSSTKKTQLQLEHLLLDLQMILNGINNYKNPKLTRMLTFKFYMPKKATELKHLQC |
| H9T | LEEELKPLEEVLNLAQSKNFHFDPRDVVSNINVFVLELKGSETTFMCEYADETATIVE |
| (L80F, R81D, L85V, | FLNRWITFCTSIISTLT |
| I86V, I92F, Q126T) | |

### B. IL-2 Mutein Fusion Proteins

In some embodiments, the IL-2 muteins for use with the subject methods can be prepared as fusion or chimeric polypeptides that include a subject IL-2 mutein and a heterologous polypeptide (i.e., a polypeptide that is not IL-2 or a mutant thereof) (see, e.g., U.S. Pat. No. 6,451,308). Exemplary heterologous polypeptides can increase the circulating half-life of the chimeric polypeptide in vivo, and may, therefore, further enhance the properties of the mutant IL-2 polypeptides. In various embodiments, the polypeptide that increases the circulating half-life may be a serum albumin, such as human serum albumin, PEG, PEG-derivatives, or the Fc region of the IgG subclass of antibodies that lacks the IgG heavy chain variable region. Exemplary Fc regions can include a mutation that inhibits complement fixation and Fc receptor binding, or it may be lytic, i.e., able to bind complement or to lyse cells via another mechanism, such as antibody-dependent complement lysis (ADCC; U.S. Ser. No. 08/355,502 filed Dec. 12, 1994).

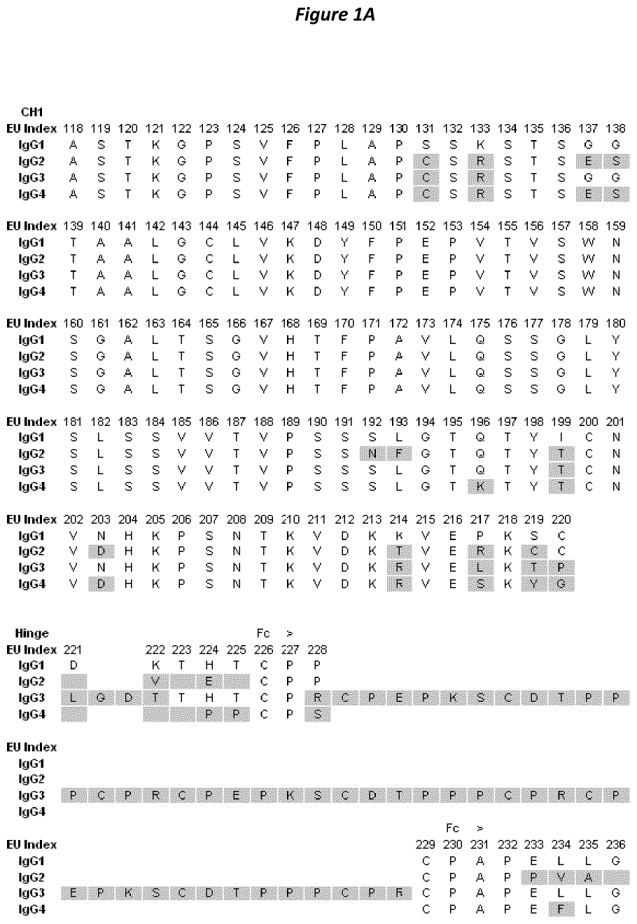

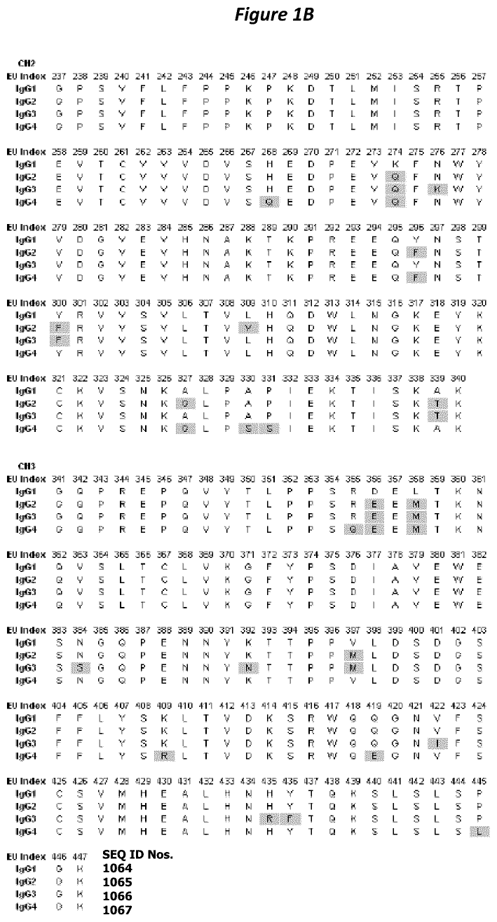

The “Fc region” can be a naturally occurring or synthetic polypeptide that is homologous to the IgG C-terminal domain produced by digestion of IgG with papain. IgG Fc has a molecular weight of approximately 50 kDa. The mutant IL-2 polypeptides can include the entire Fc region, or a smaller portion that retains the ability to extend the circulating half-life of a chimeric polypeptide of which it is a part. In addition, full-length or fragmented Fc regions can be variants of the wild-type molecule. In some embodiments, the IL-2 mutein fusion protein (e.g., an IL-2 mutein as described herein) includes an IgG1, IgG2, IgG3, or IgG4 Fc region (see, for example, sequences in FIG. 1A-1B). In some embodiments, the Fc region comprises the substitution N297A.

In some embodiments, the IL-2 mutein is linked directly or indirectly to the heterologous fusion polypeptide.

In some embodiments, the IL-2 mutein is linked directly to the Fc region. In some embodiments, the IL-2 mutein is linked to the Fc region via a linker peptide, such as GGGGS (SEQ ID NO:1000). In some embodiments, the linker is (GGGGS)n (SEQ ID NO:1062), wherein n is an integer between 1 and 10. In some embodiments, the linker is GGGGS(SEQ ID NO:1000). In some embodiments, the linker is GGGGSGGGGS (SEQ ID NO:1001). In some embodiments, the linker is GGGGSGGGGSGGGGS(SEQ ID NO:1002). In some embodiments, the linker is GGGGSGGGGSGGGGSGGGGS (SEQ ID NO:1003). In some embodiments, the linker is GGGGSGGGGSGGGGSGGGGS (SEQ ID NO:1004). In some embodiments, the linker contains one or more protease cleavage sites (e.g., is a protease cleavable linker). Linkers additionally can contain one or more protease cleavage sites or be sensitive to cleavage via oxidation and/or reduction. Peptide linkers that are susceptible to cleavage by enzymes of the complement system, urokinase, tissue plasminogen activator, trypsin, plasmin, caspases, kallikreins, cathepsins, legumain, MMPs, thrombin, urokinase-type plasminogen activator (uPA), matriptase, or another enzyme having proteolytic activity may be used in one example. According to another example, a linker may comprise disulfide bonds (for example, the disulfide bonds on a cysteine molecule). According to another example, a linker may comprise a protease-cleavable Val-Cit (VC) linker, a Phe-Arg linker, a Val-Lys linker, a Val-Ala linker, a Val-Arg linker, a Val-Leu-Lys linker, a Gly-Phe-Leu-Gly linker (SEQ ID NO: 1058), an Ala-Phe-Lys linker, a pol-L-lysine linker, a beta-Ala-Leu-Ala-Leu linker, an Arg-Arg-Ala-Leu-Ala-Leu linker (SEQ ID NO: 1059), a peptidomimetic linker, a legumain-cleavable Ala-Ala-Asn tripeptide linker, a peptide linker that is cleaved by cathepsin B and other lysosomal proteases, such as Gly-Phe-Leu-Gly (SEQ ID NO:1060) and Ala-Leu-Ala-Leu (SEQ ID NO:1061), a caspase 3 DEVD sequence, or a self-immolative linker. For example, linkers disclosed in Poreba, M, FEBS J. 287(10):1936-1969 (2020), incorporated by reference herein, are contemplated by the present disclosure. Since many tumors naturally release high levels of glutathione (a reducing agent) this can reduce the disulfide bonds with subsequent release of the cargo moiety at the site of delivery. In some embodiments, the linkers is a protease-cleavable linkers is a linker cleavable by a matrix metalloprotease (MMP). MMPs are overexpressed in situ at tumors, and linkers cleavable in such contexts are contemplated by the presentation disclosure. For example, linkers disclosed in Hsu, E. J., et aL., Nat. Commun. 12(2768):1-13 (2021), incorporated by reference herein, are contemplated by the present disclosure. In some embodiments, the MMP linker sequence is selected from the group consisting of SGARYRWLTA (SEQ ID NO: 1005), SGRSYAILTA (SEQ ID NO: 1006), SRSGRSPAIFTATG (SEQ ID NO: 1007), GSSGRSPAIFTAGS (SEQ ID NO: 1008), and SGFIANPVTA (SEQ ID NO: 1009). In some embodiments, the MMP linker sequence is SGARYRWLTA (SEQ ID NO: 1005). In some embodiments, the MMP linker sequence is SGRSYAILTA (SEQ ID NO: 1006). In some embodiments, the MMP linker sequence is SRSGRSPAIFTATG (SEQ ID NO: 1007). In some embodiments, the MMP linker sequence is GSSGRSPAIFTAGS (SEQ ID NO: 1008). In some embodiments, the MMP linker sequence is SGFIANPVTA (SEQ ID NO: 1009).

The Fc region can be “lytic” or “non-lytic,” but is typically non-lytic. A non-lytic Fc region typically lacks a high affinity Fc receptor binding site and a C′1q binding site. The high affinity Fc receptor binding site of murine IgG Fc includes the Leu residue at position 235 of IgG Fc. Thus, the Fc receptor binding site can be destroyed by mutating or deleting Leu 235. For example, substitution of Glu for Leu 235 inhibits the ability of the Fc region to bind the high affinity Fc receptor. The murine C′1q binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fc receptor binding site and a C′1q binding site. The high affinity Fc receptor binding site includes the Leu residue at position 235 of IgG Fc, and the C′1q binding site includes the Glu 318, Lys 320, and Lys 322 residues of IgG1. Lytic IgG Fc has wild-type residues or conservative amino acid substitutions at these sites. Lytic IgG Fc can target cells for antibody dependent cellular cytotoxicity or complement directed cytolysis (CDC). Appropriate mutations for human IgG are also known (see, e.g., Morrison et al., The Immunologist 2:119-124, 1994; and Brekke et al., The Immunologist 2: 125, 1994).

In other embodiments, a chimeric polypeptide including a mutant IL-2 and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the chimeric polypeptide comprises a fusion to an antibody or an antigen-binding portion thereof that disrupts the interaction between the PD-1 receptor and its ligand, PD-L1, and/or is an antibody to a component of the PD-1/PD-L1 signaling pathway. Antibodies known in the art which bind to PD-1 and disrupt the interaction between the PD-1 and its ligand, PD-L1, and stimulate an anti-tumor immune response, are suitable for use in the chimeric polypeptides disclosed herein. In some embodiments, the antibody or antigen-binding portion thereof binds specifically to PD-1. For example, antibodies that target PD-1 and which can find used in the present invention include, e.g., but are not limited to nivolumab (BMS-936558, Bristol-Myers Squibb), pembrolizumab (lambrolizumab, MK03475 or MK-3475, Merck), humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (cemiplimab, Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)—BioXcell cat #BP0146. Other suitable antibodies include anti-PD-1 antibodies disclosed in U.S. Pat. No. 8,008,449, herein incorporated by reference. In some embodiments, the antibody or antigen-binding portion thereof binds specifically to PD-L1 and inhibits its interaction with PD-1, thereby increasing immune activity. Any antibodies known in the art which bind to PD-L1 and disrupt the interaction between the PD-1 and PD-L1, and stimulates an anti-tumor immune response, are suitable for use in the chimeric polypeptides disclosed herein. For example, antibodies that target PD-L1 and are in clinical trials, include BMS-936559 (Bristol-Myers Squibb) and MPDL3280A (Genentech). Other suitable antibodies that target PD-L1 are disclosed in U.S. Pat. No. 7,943,743, herein incorporated by reference. It will be understood by one of ordinary skill that any antibody which binds to PD-1 or PD-L1, disrupts the PD-1/PD-L1 interaction, and stimulates an anti-tumor immune response, is suitable for use in the chimeric polypeptides disclosed herein. In some embodiments, the chimeric polypeptide comprises a fusion to an anti-PD-1 antibody. In some embodiments, the chimeric polypeptide comprises a fusion to an anti-PD-L1 antibody.

Exemplary IL-2 fusion proteins include those listed in the table below:

TABLE 3
IL-2 Extended Half-Life Fusion Amino Acid Sequences

| SEQ ID NO: | |
|---|---|
| (Information) | Amino acid sequence |
| SEQ ID NO: 22 | APTSSSTKKTQLQLEHLLLDLQMILNGINNYKNPKLTRMLTFKFYMPKKATELKH |
| hIL2-Fc | LQCLEEELKPLEEVLNLAQSKNFHLRPRDLISNINVIVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 23 | APTSSSTKKTQLQLEHLLLDLQMILNGINNYKNPKLTRMLTFKFYMPKKATELKH |
| hIL-2-Fc | LQCLEEELKPLEEVLNLAQSKNFHLRPRDLISNINVIVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 24 | APTSSSTKKTQLQLEHLLLDLQMILNGINNYKNPKLTRMLTFKFYMPKKATELKH |
| H9-Fc | LQCLEEELKPLEEVLNLAQSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 25 | APTSSSTKKTQLQLEHLLLDLQMILNGINNYKNPKLTRMLTFKFYMPKKATELKH |
| H9-Fc | LQCLEEELKPLEEVLNLAQSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 26 and 27 | Gene 1: |
| H9-Fc (KIH) | APTSSSTKKTQLQLEHLLLDLQMILNGINNYKNPKLTRMLTFKFYMPKKATELKHL |
| | QCLEEELKPLEEVLNLAQSKNFHFDPRDVVSNINVFVLELKGSETTFMCEYADETA |
| | TIVEFLNRWITFCQSIISTLTGGGGSGGGGSGGGGSDKTHTCPPCPAPELLGGPSV |
| | FLFPPKPKDTLMISRTPEVTCVVVDVSHEDPEVKFNWYVDGVEVHNAKTKPREEQY |
| | ASTYRVVSVLTVLHQDWLNGKEYKCKVSNKALPAPIEKTISKAKGQPREPQVYTLP |
| | PCREEMTKNQVSLWCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 26) |
| | Gene 2: |
| | DKTHTCPPCPAPELLGGPSVFLFPPKPKDTLMISRTPEVTCVVVDVSHEDPEVKF |
| | NWYVDGVEVHNAKTKPREEQYASTYRVVSVLTVLHQDWLNGKEYKCKVSNKALPA |
| | PIEKTISKAKGQPREPQVCTLPPSREEMTKNQVSLSCAVKGFYPSDIAVEWESNG |
| | QPENNYKTTPPVLDSDGSFFLVSKLTVDKSRWQQGNVFSCSVMHEALHNHYTQKS |
| | LSLSPGK (SEQ ID NO: 27) |
| SEQ ID NO: | Gene 1: |
| 28 and 27 | DKTHTCPPCPAPELLGGPSVFLFPPKPKDTLMISRTPEVTCVVVDVSHEDPEVKFN |
| Fc-H9 (KIH) | WYVDGVEVHNAKTKPREEQYASTYRVVSVLTVLHQDWLNGKEYKCKVSNKALPAPI |
| | EKTISKAKGQPREPQVYTLPPCREEMTKNQVSLWCLVKGFYPSDIAVEWESNGQPE |
| | NNYKTTPPVLDSDGSFFLYSKLTVDKSRWQQGNVFSCSVMHEALHNHYTQKSLSLS |
| | PGGGGGSGGGGSGGGGSAPTSSSTKKTQLQLEHLLLDLQMILNGINNYKNPKLTRM |
| | LTFKFYMPKKATELKHLQCLEEELKPLEEVLNLAQSKNFHFDPRDVVSNINVFVLE |
| | LKGSETTFMCEYADETATIVEFLNRWITFCQSIISTLT (SEQ ID |
| | NO: 28 |
| | Gene 2: |
| | DKTHTCPPCPAPELLGGPSVFLFPPKPKDTLMISRTPEVTCVVVDVSHEDPEVKF |
| | NWYVDGVEVHNAKTKPREEQYASTYRVVSVLTVLHQDWLNGKEYKCKVSNKALPA |
| | PIEKTISKAKGQPREPQVCTLPPSREEMTKNQVSLSCAVKGFYPSDIAVEWESNG |
| | QPENNYKTTPPVLDSDGSFFLVSKLTVDKSRWQQGNVFSCSVMHEALHNHYTQKS |
| | LSLSPGK (SEQ ID NO: 27) |
| SEQ ID NO: 29 | APTSSSTKKTQLQLEHLLLDLQMILNGINNYKNPKLTRMLTAKFAMPKKATELKH |
| H9-FYAA-Fc | LQCLEEELKPLEEVLNLAQSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 30 | APTSSSTKKTQLQLEHLLLDLQMILNGINNYKNPKLTRMLTAKFAMPKKATELKH |
| H9FYAA-Fc | LQCLEEELKPLEEVLNLAQSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 31 | APTSSSTKKTQLQLEHLLLDLQMILNGINNYKNPKLTRMLTAKFYMPKKATELKH |
| H9-FEAA-Fc | LQCLEEALKPLEEVLNLAQSKNFHFDPRDVVSNINVFVLELKGSETTFMCEYADE |
| (also referred to | TATIVEFLNRWITFCQSIISTLTGGGGSGGGGSGGGGSDKTHTCPPCPAPELLGG |
| as MDNA19 or | PSVFLFPPKPKDTLMISRTPEVTCVVVDVSHEDPEVKFNWYVDGVEVHNAKTKPR |
| MDNA109FEAA-Fc) | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 32 | APTSSSTKKTQLQLEHLLLDLQMILNGINNYKNPKLTRMLTAKFYMPKKATELKH |
| H9FEAA-Fc | LQCLEEALKPLEEVLNLAQSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 33 | APTSSSTKKTQLQLEHLLLDLQMILNGINNYKNPKLTRMLTAKFAMPKKATELKH |
| H9-FYEAAA-Fc | LQCLEEALKPLEEVLNLAQSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 34 | APTSSSTKKTQLQLEHLLLDLQMILNGINNYKNPKLTRMLTFKFYMPKKATELKH |
| H9D10-Fc | LQCLEEELKPLEEVLNLAHSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 35 | APTSSSTKKTQLQLEHLLLDLQMILNGINNYKNPKLTRMLTFKFYMPKKATELKH |
| H9E10-Fc | LQCLEEELKPLEEVLNLASSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 36 | APTSSSTKKTQLQLEHLLLDLQMILNGINNYKNPKLTRMLTFKFYMPKKATELKH |
| H9G8-Fc | LQCLEEELKPLEEVLNLANSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 37 | APTSSSTKKTQLQLEHLLLDLQMILNGINNYKNPKLTRMLTFKFYMPKKATELKH |
| H9B1-Fc | LQCLEEELKPLEEVLNLANSKNFHFDPRDVVSNVNVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK* |
| SEQ ID NO: 38 | DAHKSEVAHRFKDLGEENFKALVLIAFAQYLQQCPFEDHVKLVNEVTEFAKTCVA |
| Albumin-H9 | DESAENCDKSLHTLFGDKLCTVATLRETYGEMADCCAKQEPERNECFLQHKDDNP |
| | NLPRLVRPEVDVMCTAFHDNEETFLKKYLYEIARRHPYFYAPELLFFAKRYKAAF |
| | TECCQAADKAACLLPKLDELRDEGKASSAKQRLKCASLQKFGERAFKAWAVARLS |
| | QRFPKAEFAEVSKLVTDLTKVHTECCHGDLLECADDRADLAKYICENQDSISSKL |
| | KECCEKPLLEKSHCIAEVENDEMPADLPSLAADFVESKDVCKNYAEAKDVFLGMF |
| | LYEYARRHPDYSVVLLLRLAKTYETTLEKCCAAADPHECYAKVFDEFKPLVEEPQ |
| | NLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCFSALEVDE |
| | TYVPKEFNAETFTFHADICTLSEKERQIKKQTALVELVKHKPKATKEQLKAVMDD |
| | FAAFVEKCCKADDKETCFAEEGKKLVAASQAALGLGGGGSGGGGSGGGGSAPTSS |
| | STKKTQLQLEHLLLDLQMILNGINNYKNPKLTRMLTFKFYMPKKATELKHLQCLE |
| | EELKPLEEVLNLAQSKNFHFDPRDVVSNINVFVLELKGSETTFMCEYADETATIV |
| | EFLNRWITFCQSIISTLT* |
| SEQ ID NO: 39 | DAHKSEVAHRFKDLGEENFKALVLIAFAQYLQQCPFEDHVKLVNEVTEFAKTCVA |
| Albumin-H9FYAA | DESAENCDKSLHTLFGDKLCTVATLRETYGEMADCCAKQEPERNECFLQHKDDNP |
| | NLPRLVRPEVDVMCTAFHDNEETFLKKYLYEIARRHPYFYAPELLFFAKRYKAAF |
| | TECCQAADKAACLLPKLDELRDEGKASSAKQRLKCASLQKFGERAFKAWAVARLS |
| | QRFPKAEFAEVSKLVTDLTKVHTECCHGDLLECADDRADLAKYICENQDSISSKL |
| | KECCEKPLLEKSHCIAEVENDEMPADLPSLAADFVESKDVCKNYAEAKDVFLGMF |
| | LYEYARRHPDYSVVLLLRLAKTYETTLEKCCAAADPHECYAKVFDEFKPLVEEPQ |
| | NLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCFSALEVDE |
| | TYVPKEFNAETFTFHADICTLSEKERQIKKQTALVELVKHKPKATKEQLKAVMDD |
| | FAAFVEKCCKADDKETCFAEEGKKLVAASQAALGLGGGGSGGGGSGGGGSAPTSS |
| | STKKTQLQLEHLLLDLQMILNGINNYKNPKLTRMLTAKFAMPKKATELKHLQCLE |
| | EELKPLEEVLNLAQSKNFHFDPRDVVSNINVFVLELKGSETTFMCEYADETATIV |
| | EFLNRWITFCQSIISTLT |
| SEQ ID NO: 40 | DAHKSEVAHRFKDLGEENFKALVLIAFAQYLQQCPFEDHVKLVNEVTEFAKTCVA |
| Albumin-H9FEAA | DESAENCDKSLHTLFGDKLCTVATLRETYGEMADCCAKQEPERNECFLQHKDDNP |
| | NLPRLVRPEVDVMCTAFHDNEETFLKKYLYEIARRHPYFYAPELLFFAKRYKAAF |
| | TECCQAADKAACLLPKLDELRDEGKASSAKQRLKCASLQKFGERAFKAWAVARLS |
| | QRFPKAEFAEVSKLVTDLTKVHTECCHGDLLECADDRADLAKYICENQDSISSKL |
| | KECCEKPLLEKSHCIAEVENDEMPADLPSLAADFVESKDVCKNYAEAKDVFLGMF |
| | LYEYARRHPDYSVVLLLRLAKTYETTLEKCCAAADPHECYAKVFDEFKPLVEEPQ |
| | NLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCFSALEVDE |
| | TYVPKEFNAETFTFHADICTLSEKERQIKKQTALVELVKHKPKATKEQLKAVMDD |
| | FAAFVEKCCKADDKETCFAEEGKKLVAASQAALGLGGGGSGGGGSGGGGSAPTSS |
| | STKKTQLQLEHLLLDLQMILNGINNYKNPKLTRMLTAKFYMPKKATELKHLQCLE |
| | EALKPLEEVLNLAQSKNFHFDPRDVVSNINVFVLELKGSETTFMCEYADETATIV |
| | EFLNRWITFCQSIISTLT |
| SEQ ID NO: 41 | APTSSSTKKTQLQLEHLLLDLQMILNGINNYKNPKLTRMLTFKFYMPKKATELKH |
| H9D10-Albumin | LQCLEEELKPLEEVLNLAHSKNFHFDPRDVVSNINVFVLELKGSETTFMCEYADE |
| | TATIVEFLNRWITFCQSIISTLTGGGGSGGGGSGGGGSDAHKSEVAHRFKDLGEE |
| | NFKALVLIAFAQYLQQCPFEDHVKLVNEVTEFAKTCVADESAENCDKSLHTLFGD |
| | KLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL |
| SEQ ID NO: 42 | APTSSSTKKTQLQLEHLLLDLQMILNGINNYKNPKLTRMLTAKFYMPKKATELKH |
| H9D10FEAA-Albumin | LQCLEEALKPLEEVLNLAHSKNFHFDPRDVVSNINVFVLELKGSETTFMCEYADE |
| | TATIVEFLNRWITFCQSIISTLTGGGGSGGGGSGGGGSDAHKSEVAHRFKDLGEE |
| | NFKALVLIAFAQYLQQCPFEDHVKLVNEVTEFAKTCVADESAENCDKSLHTLFGD |
| | KLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL |
| SEQ ID NO: 43 | APTSSSTKKTQLQLEHLLLDLQMILNGINNYKNPKLTRMLTFKFYMPKKATELKH |
| H9E10-Albumin | LQCLEEELKPLEEVLNLASSKNFHFDPRDVVSNINVFVLELKGSETTFMCEYADE |
| | TATIVEFLNRWITFCQSIISTLTGGGGSGGGGSGGGGSDAHKSEVAHRFKDLGEE |
| | NFKALVLIAFAQYLQQCPFEDHVKLVNEVTEFAKTCVADESAENCDKSLHTLFGD |
| | KLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL |
| SEQ ID NO: 44 | APTSSSTKKTQLQLEHLLLDLQMILNGINNYKNPKLTRMLTFKFYMPKKATELKH |
| H9G8-Albumin | LQCLEEELKPLEEVLNLANSKNFHFDPRDVVSNINVFVLELKGSETTFMCEYADE |
| | TATIVEFLNRWITFCQSIISTLTGGGGSGGGGSGGGGSDAHKSEVAHRFKDLGEE |
| | NFKALVLIAFAQYLQQCPFEDHVKLVNEVTEFAKTCVADESAENCDKSLHTLFGD |
| | KLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL |
| SEQ ID NO: 45 | APTSSSTKKTQLQLEHLLLDLQMILNGINNYKNPKLTRMLTFKFYMPKKATELKH |
| H9B1-Albumin | LQCLEEELKPLEEVLNLANSKNFHFDPRDVVSNVNVFVLELKGSETTFMCEYADE |
| | TATIVEFLNRWITFCQSIISTLTGGGGSGGGGSGGGGSDAHKSEVAHRFKDLGEE |
| | NFKALVLIAFAQYLQQCPFEDHVKLVNEVTEFAKTCVADESAENCDKSLHTLFGD |
| | KLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL |
| SEQ ID NO: 46 | APTSSSTKKTQLQLEHLLLDLQMILNGINNYKNPKLTRMLTAKFYMPKKATELKH |
| H9FEAA-Albumin | LQCLEEALKPLEEVLNLAQSKNFHFDPRDVVSNINVFVLELKGSETTFMCEYADE |
| | TATIVEFLNRWITFCQSIISTLTGGGGSGGGGSGGGGSDAHKSEVAHRFKDLGEE |
| | NFKALVLIAFAQYLQQCPFEDHVKLVNEVTEFAKTCVADESAENCDKSLHTLFGD |
| | KLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL |
| SEQ ID NO: 47 | DAHKSEVAHRFKDLGEENFKALVLIAFAQYLQQCPFEDHVKLVNEVTEFAKTCVA |
| Albumin-H9D10 | DESAENCDKSLHTLFGDKLCTVATLRETYGEMADCCAKQEPERNECFLQHKDDNP |
| | NLPRLVRPEVDVMCTAFHDNEETFLKKYLYEIARRHPYFYAPELLFFAKRYKAAF |
| | TECCQAADKAACLLPKLDELRDEGKASSAKQRLKCASLQKFGERAFKAWAVARLS |
| | QRFPKAEFAEVSKLVTDLTKVHTECCHGDLLECADDRADLAKYICENQDSISSKL |
| | KECCEKPLLEKSHCIAEVENDEMPADLPSLAADFVESKDVCKNYAEAKDVFLGMF |
| | LYEYARRHPDYSVVLLLRLAKTYETTLEKCCAAADPHECYAKVFDEFKPLVEEPQ |
| | NLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCFSALEVDE |
| | TYVPKEFNAETFTFHADICTLSEKERQIKKQTALVELVKHKPKATKEQLKAVMDD |
| | FAAFVEKCCKADDKETCFAEEGKKLVAASQAALGLGGGGSGGGGSGGGGSAPTSS |
| | STKKTQLQLEHLLLDLQMILNGINNYKNPKLTRMLTFKFYMPKKATELKHLQCLE |
| | EELKPLEEVLNLAHSKNFHFDPRDVVSNINVFVLELKGSETTFMCEYADETATIV |
| | EFLNRWITFCQSIISTLT |
| SEQ ID NO: 48 | DAHKSEVAHRFKDLGEENFKALVLIAFAQYLQQCPFEDHVKLVNEVTEFAKTCVA |
| Albumin-H9D10 FEAA | DESAENCDKSLHTLFGDKLCTVATLRETYGEMADCCAKQEPERNECFLQHKDDNP |
| | NLPRLVRPEVDVMCTAFHDNEETFLKKYLYEIARRHPYFYAPELLFFAKRYKAAF |
| | TECCQAADKAACLLPKLDELRDEGKASSAKQRLKCASLQKFGERAFKAWAVARLS |
| | QRFPKAEFAEVSKLVTDLTKVHTECCHGDLLECADDRADLAKYICENQDSISSKL |
| | KECCEKPLLEKSHCIAEVENDEMPADLPSLAADFVESKDVCKNYAEAKDVFLGMF |
| | LYEYARRHPDYSVVLLLRLAKTYETTLEKCCAAADPHECYAKVFDEFKPLVEEPQ |
| | NLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCFSALEVDE |
| | TYVPKEFNAETFTFHADICTLSEKERQIKKQTALVELVKHKPKATKEQLKAVMDD |
| | FAAFVEKCCKADDKETCFAEEGKKLVAASQAALGLGGGGSGGGGSGGGGSAPTSS |
| | STKKTQLQLEHLLLDLQMILNGINNYKNPKLTRMLTAKFYMPKKATELKHLQCLE |
| | EALKPLEEVLNLAHSKNFHFDPRDVVSNINVFVLELKGSETTFMCEYADETATIV |
| | EFLNRWITFCQSIISTLT |
| SEQ ID NO: 49 | DAHKSEVAHRFKDLGEENFKALVLIAFAQYLQQCPFEDHVKLVNEVTEFAKTCVA |
| Albumin-H9E10 | DESAENCDKSLHTLFGDKLCTVATLRETYGEMADCCAKQEPERNECFLQHKDDNP |
| | NLPRLVRPEVDVMCTAFHDNEETFLKKYLYEIARRHPYFYAPELLFFAKRYKAAF |
| | TECCQAADKAACLLPKLDELRDEGKASSAKQRLKCASLQKFGERAFKAWAVARLS |
| | QRFPKAEFAEVSKLVTDLTKVHTECCHGDLLECADDRADLAKYICENQDSISSKL |
| | KECCEKPLLEKSHCIAEVENDEMPADLPSLAADFVESKDVCKNYAEAKDVFLGMF |
| | LYEYARRHPDYSVVLLLRLAKTYETTLEKCCAAADPHECYAKVFDEFKPLVEEPQ |
| | NLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCFSALEVDE |
| | TYVPKEFNAETFTFHADICTLSEKERQIKKQTALVELVKHKPKATKEQLKAVMDD |
| | FAAFVEKCCKADDKETCFAEEGKKLVAASQAALGLGGGGSGGGGSGGGGSAPTSS |
| | STKKTQLQLEHLLLDLQMILNGINNYKNPKLTRMLTFKFYMPKKATELKHLQCLE |
| | EELKPLEEVLNLASSKNFHFDPRDVVSNINVFVLELKGSETTFMCEYADETATIV |
| | EFLNRWITFCQSIISTLT |
| SEQ ID NO: 50 | DAHKSEVAHRFKDLGEENFKALVLIAFAQYLQQCPFEDHVKLVNEVTEFAKTCVA |
| Albumin-H9G8 | DESAENCDKSLHTLFGDKLCTVATLRETYGEMADCCAKQEPERNECFLQHKDDNP |
| | NLPRLVRPEVDVMCTAFHDNEETFLKKYLYEIARRHPYFYAPELLFFAKRYKAAF |
| | TECCQAADKAACLLPKLDELRDEGKASSAKQRLKCASLQKFGERAFKAWAVARLS |
| | QRFPKAEFAEVSKLVTDLTKVHTECCHGDLLECADDRADLAKYICENQDSISSKL |
| | KECCEKPLLEKSHCIAEVENDEMPADLPSLAADFVESKDVCKNYAEAKDVFLGMF |
| | LYEYARRHPDYSVVLLLRLAKTYETTLEKCCAAADPHECYAKVFDEFKPLVEEPQ |
| | NLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCFSALEVDE |
| | TYVPKEFNAETFTFHADICTLSEKERQIKKQTALVELVKHKPKATKEQLKAVMDD |
| | FAAFVEKCCKADDKETCFAEEGKKLVAASQAALGLGGGGSGGGGSGGGGSAPTSS |
| | STKKTQLQLEHLLLDLQMILNGINNYKNPKLTRMLTFKFYMPKKATELKHLQCLE |
| | EELKPLEEVLNLANSKNFHFDPRDVVSNINVFVLELKGSETTFMCEYADETATIV |
| | EFLNRWITFCQSIISTLT |
| SEQ ID NO: 51 | DAHKSEVAHRFKDLGEENFKALVLIAFAQYLQQCPFEDHVKLVNEVTEFAKTCVA |
| Albumin-H9B1 | DESAENCDKSLHTLFGDKLCTVATLRETYGEMADCCAKQEPERNECFLQHKDDNP |
| | NLPRLVRPEVDVMCTAFHDNEETFLKKYLYEIARRHPYFYAPELLFFAKRYKAAF |
| | TECCQAADKAACLLPKLDELRDEGKASSAKQRLKCASLQKFGERAFKAWAVARLS |
| | QRFPKAEFAEVSKLVTDLTKVHTECCHGDLLECADDRADLAKYICENQDSISSKL |
| | KECCEKPLLEKSHCIAEVENDEMPADLPSLAADFVESKDVCKNYAEAKDVFLGMF |
| | LYEYARRHPDYSVVLLLRLAKTYETTLEKCCAAADPHECYAKVFDEFKPLVEEPQ |
| | NLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCFSALEVDE |
| | TYVPKEFNAETFTFHADICTLSEKERQIKKQTALVELVKHKPKATKEQLKAVMDD |
| | FAAFVEKCCKADDKETCFAEEGKKLVAASQAALGLGGGGSGGGGSGGGGSAPTSS |
| | STKKTQLQLEHLLLDLQMILNGINNYKNPKLTRMLTFKFYMPKKATELKHLQCLE |
| | EELKPLEEVLNLANSKNFHFDPRDVVSNVNVFVLELKGSETTFMCEYADETATIV |
| | EFLNRWITFCQSIISTLT |
| SEQ ID NO: 52 | APTSSSTKKTQLQLEHLLLDLQMILNGINNYKNPKLTRMLTAKFYMPKKATELKH |
| MDNA109FEAA-C125S- | LQCLEEALKPLEEVLNLAQSKNFHFDPRDVVSNINVFVLELKGSETTFMCEYADE |
| Albumin | TATIVEFLNRWITFSQSIISTLTGGGGSGGGGSGGGGSDAHKSEVAHRFKDLGEE |
| | NFKALVLIAFAQYLQQCPFEDHVKLVNEVTEFAKTCVADESAENCDKSLHTLFGD |
| | KLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL* |
| SEQ ID NO: 53 | APTSSSTKKTQLQLEHLLLDLQMILNGINNYKNPKLTRMLTAKFYMPKKATELKHL |
| MDNA11 | QCLEEALKPLEEVLNLAQSKNFHFDPRDVVSNINVFVLELKGSETTFMCEYADETA |
| (MDNA109FEAA- | TIVEFLNRWITFCQSIISTLTGGGGSGGGGSGGGGSDAHKSEVAHRFKDLGEENFK |
| Albumin) | ALVLIAFAQYLQQCPFEDHVKLVNEVTEFAKTCVADESAENCDKSLHTLFGDKLCT |
| | VATLRETYGEMADCCAKQEPERNECFLQHKDDNPNLPRLVRPEVDVMCTAFHDNEE |
| | TFLKKYLYEIARRHPYFYAPELLFFAKRYKAAFTECCQAADKAACLLPKLDELRDE |
| | GKASSAKQRLKCASLQKFGERAFKAWAVARLSQRFPKAEFAEVSKLVTDLTKVHTE |
| | CCHGDLLECADDRADLAKYICENQDSISSKLKECCEKPLLEKSHCIAEVENDEMPA |
| | DLPSLAADFVESKDVCKNYAEAKDVFLGMFLYEYARRHPDYSVVLLLRLAKTYETT |
| | LEKCCAAADPHECYAKVFDEFKPLVEEPQNLIKQNCELFEQLGEYKFQNALLVRYT |
| | KKVPQVSTPTLVEVSRNLGKVGSKCCKHPEAKRMPCAEDYLSVVLNQLCVLHEKTP |
| | VSDRVTKCCTESLVNRRPCFSALEVDETYVPKEFNAETFTFHADICTLSEKERQIK |
| | KQTALVELVKHKPKATKEQLKAVMDDFAAFVEKCCKADDKETCFAEEGKKLVAASQ |
| | AALGL* |
| SEQ ID NO: 54 | APTSSSTKKTQLQLEHLLLDLQMILNGINNYKNPKLTRMLTFKFYMPKKATELKH |
| MDNA109-Fc (2:1) | LQCLEEELKPLEEVLNLAQSKNFHFDPRDVVSNINVFVLELKGSETTFMCEYADE |
| | TATIVEFLNRWITFCQSIISTLTGGGGSGGGGSGGGGSDKTHTCPPCPAPELLGG |
| | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| SEQ ID NO: 55 | DKTHTCPPCPAPELLGGPSVFLFPPKPKDTLMISRTPEVTCVVVDVSHEDPEVKF |
| Fc-MDNA109 (1:2) | NWYVDGVEVHNAKTKPREEQYASTYRVVSVLTVLHQDWLNGKEYKCKVSNKALPA |
| | PIEKTISKAKGQPREPQVYTLPPSREEMTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGGGGGSGGGGSGGGGSAPTSSSTKKTQLQLEHLLLDLQMILNGINNYKNP |
| | KLTRMLTFKFYMPKKATELKHLQCLEEELKPLEEVLNLAQSKNFHFDPRDVVSNI |
| | NVFVLELKGSETTFMCEYADETATIVEFLNRWITFCQSIISTLT |
| SEQ ID NO: 56 and 27 | Gene 1: |
| Fc-MDNA109 (1:1) | DKTHTCPPCPAPELLGGPSVFLFPPKPKDTLMISRTPEVTCVVVDVSHEDPEVKEN |
| KIH | WYVDGVEVHNAKTKPREEQYASTYRVVSVLTVLHQDWLNGKEYKCKVSNKALPAPI |
| (L80F, R81D, L85V, | EKTISKAKGQPREPQVYTLPPCREEMTKNQVSLWCLVKGFYPSDIAVEWESNGQPE |
| I86V, I92F) | NNYKTTPPVLDSDGSFFLYSKLTVDKSRWQQGNVFSCSVMHEALHNHYTQKSLSLS |
| | PGGGGGSGGGGSGGGGSAPTSSSTKKTQLQLEHLLLDLQMILNGINNYKNPKLTRM |
| | LTFKFYMPKKATELKHLQCLEEELKPLEEVLNLAQSKNFHFDPRDVVSNINVFVLE |
| | LKGSETTFMCEYADETATIVEFLNRWITFCQSIISTLT (SEQ ID NO: 56) |
| | Gene 2: |
| | DKTHTCPPCPAPELLGGPSVFLFPPKPKDTLMISRTPEVTCVVVDVSHEDPEVKF |
| | NWYVDGVEVHNAKTKPREEQYASTYRVVSVLTVLHQDWLNGKEYKCKVSNKALPA |
| | PIEKTISKAKGQPREPQVCTLPPSREEMTKNQVSLSCAVKGFYPSDIAVEWESNG |
| | QPENNYKTTPPVLDSDGSFFLVSKLTVDKSRWQQGNVFSCSVMHEALHNHYTQKS |
| | LSLSPGK (SEQ ID NO: 27) |
| SEQ ID NO: 57 | APTSSSTKKTQLQLEHLLLDLQMILNGINNYKNPKLTRMLTFKFYMPKKATELKH |
| MDNA109-Alb | LQCLEEELKPLEEVLNLAQSKNFHFDPRDVVSNINVFVLELKGSETTFMCEYADE |
| (L80F, R81D, L85V, | TATIVEFLNRWITFCQSIISTLTGGGGSGGGGSGGGGSDAHKSEVAHRFKDLGEE |
| I86V, I92F) | NFKALVLIAFAQYLQQCPFEDHVKLVNEVTEFAKTCVADESAENCDKSLHTLFGD |
| | KLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL |
| SEQ ID NO: 58 | DKTHTCPPCPAPELLGGPSVFLFPPKPKDTLMISRTPEVTCVVVDVSHEDPEVKF |
| Fc -MDNA109FEAA | NWYVDGVEVHNAKTKPREEQYASTYRVVSVLTVLHQDWLNGKEYKCKVSNKALPA |
| (1:2) | PIEKTISKAKGQPREPQVYTLPPSREEMTKNQVSLTCLVKGFYPSDIAVEWESNG |
| (F42A, E62A, L80F, | QPENNYKTTPPVLDSDGSFFLYSKLTVDKSRWQQGNVFSCSVMHEALHNHYTQKS |
| R81D, L85V, I86V, | LSLSPGGGGGSGGGGSGGGGSAPTSSSTKKTQLQLEHLLLDLQMILNGINNYKNP |
| I92F) | KLTRMLTAKFYMPKKATELKHLQCLEEALKPLEEVLNLAQSKNFHFDPRDVVSNI |
| | NVFVLELKGSETTFMCEYADETATIVEFLNRWITFCQSIISTLT |
| SEQ ID NO: 59 and 27 | Gene 1: |
| KIH Fc -MDNA109FEAA | DKTHTCPPCPAPELLGGPSVFLFPPKPKDTLMISRTPEVTCVVVDVSHEDPEVKEN |
| (1:1) | WYVDGVEVHNAKTKPREEQYASTYRVVSVLTVLHQDWLNGKEYKCKVSNKALPAPI |
| (F42A, E62A, L80F, | EKTISKAKGQPREPQVYTLPPSREEMTKNQVSLTCLVKGFYPSDIAVEWESNGQPE |
| R81D, L85V, I86V, | NNYKTTPPVLDSDGSFFLYSKLTVDKSRWQQGNVFSCSVMHEALHNHYTQKSLSLS |
| I92F) | PGKGGGGSGGGGSGGGGSAPTSSSTKKTQLQLEHLLLDLQMILNGINNYKNPKLTR |
| | MLTAKFYMPKKATELKHLQCLEEALKPLEEVLNLAQSKNFHFDPRDVVSNINVFVL |
| | ELKGSETTFMCEYADETATIVEFLNRWITFCQSIISTLT (SEQ ID NO: 59) |
| | Gene 2: |
| | DKTHTCPPCPAPELLGGPSVFLFPPKPKDTLMISRTPEVTCVVVDVSHEDPEVKF |
| | NWYVDGVEVHNAKTKPREEQYASTYRVVSVLTVLHQDWLNGKEYKCKVSNKALPA |
| | PIEKTISKAKGQPREPQVCTLPPSREEMTKNQVSLSCAVKGFYPSDIAVEWESNG |
| | QPENNYKTTPPVLDSDGSFFLVSKLTVDKSRWQQGNVFSCSVMHEALHNHYTQKS |
| | LSLSPGK (SEQ ID NO: 27) |
| SEQ ID NO: 60 | APTSSSTKKTQLQLEHLLLDLQMILNGINNYKNPKLTWMLTFKFYMPKKATELKH |
| MDNA109R38W-Fc | LQCLEEELKPLEEVLNLAQSKNFHFDPRDVVSNINVFVLELKGSETTFMCEYADE |
| (2:1) | TATIVEFLNRWITFCQSIISTLTGGGGSGGGGSGGGGSDKTHTCPPCPAPELLGG |
| (R38W, L80F, R81D, | PSVFLFPPKPKDTLMISRTPEVTCVVVDVSHEDPEVKFNWYVDGVEVHNAKTKPR |
| L85V, I86V, I92F) | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| SEQ ID NO: 61 | APTSSSTKKTQLQLEHLLLDLQMILNGINNYKNPKLTRMLTAKFYMPKKATELKH |
| MDNA109G8-FE-Fc | LQCLEEALKPLEEVLNLANSKNFHFDPRDVVSNINVFVLELKGSETTFMCEYADE |
| (F42A, E62A, Q74N, | TATIVEFLNRWITFCQSIISTLTGGGGSGGGGSGGGGSDKTHTCPPCPAPELLGG |
| L80F, R81D, L85V, | PSVFLFPPKPKDTLMISRTPEVTCVVVDVSHEDPEVKFNWYVDGVEVHNAKTKPR |
| I86V, I92F) | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| SEQ ID NO: 62 | APTSSSTKKTQLQLEHLLLDLQMILNGINNYKNPKLTRMLTAKFAMPKKATELKH |
| MDNA109G8-FEY-Fc | LQCLEEELKPLEEVLNLANSKNFHFDPRDVVSNINVFVLELKGSETTFMCEYADE |
| (F42A, Y45A, Q74N, | TATIVEFLNRWITFCQSIISTLTGGGGSGGGGSGGGGSDKTHTCPPCPAPELLGG |
| L80F, R81D, L85V, | PSVFLFPPKPKDTLMISRTPEVTCVVVDVSHEDPEVKFNWYVDGVEVHNAKTKPR |
| I86V, I92F) | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| SEQ ID NO: 63 | APTSSSTKKTQLQLEHLLLDLQMILNGINNYKNPKLTWMLTAKFYMPKKATELKH |
| MDNA109G8-R38-Fc | LQCLEEALKPLEEVLNLANSKNFHFDPRDVVSNINVFVLELKGSETTFMCEYADE |
| (Q74N, L80F, R81D, | TATIVEFLNRWITFCQSIISTLTGGGGSGGGGSGGGGSDKTHTCPPCPAPELLGG |
| L85V, I86V, I92F) | PSVFLFPPKPKDTLMISRTPEVTCVVVDVSHEDPEVKFNWYVDGVEVHNAKTKPR |
| | EEQYASTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| SEQ ID NO: 64 | APTSSSTKKTQLQLEHLLLDLQMILNGINNYKNPKLTWMLTAKFYMPKKATELKH |
| MDNA109G8-R38-FE-Fc | LQCLEEALKPLEEVLNLANSKNFHFDPRDVVSNINVFVLELKGSETTFMCEYADE |
| (R38-FE) (F42A, | TATIVEFLNRWITFCQSIISTLTGGGGSGGGGSGGGGSDKTHTCPPCPAPELLGG |
| E62A, Q74N, L80F, | PSVFLFPPKPKDTLMISRTPEVTCVVVDVSHEDPEVKFNWYVDGVEVHNAKTKPR |
| R81D, L85V, 186V, | EEQYASTYRVVSVLTVLHQDWLNGKEYKCKVSNKALPAPIEKTISKAKGQPREPQ |
| I92F) | VYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| SEQ ID NO: 65 | DKTHTCPPCPAPELLGGPSVFLFPPKPKDTLMISRTPEVTCVVVDVSHEDPEVKF |
| Fc-MDNA109FEY | NWYVDGVEVHNAKTKPREEQYASTYRVVSVLTVLHQDWLNGKEYKCKVSNKALPA |
| (F42A, Y45A, L80F, | PIEKTISKAKGQPREPQVYTLPPSREEMTKNQVSLTCLVKGFYPSDIAVEWESNG |
| R81D, L85V, I86V, | QPENNYKTTPPVLDSDGSFFLYSKLTVDKSRWQQGNVFSCSVMHEALHNHYTQKS |
| I92F) | LSLSPGGGGGSGGGGSGGGGSAPTSSSTKKTQLQLEHLLLDLQMILNGINNYKNP |
| | KLTRMLTAKFAMPKKATELKHLQCLEEELKPLEEVLNLAQSKNFHFDPRDVVSNI |
| | NVFVLELKGSETTFMCEYADETATIVEFLNRWITFCQSIISTLT |
| SEQ ID NO: 66 | APTSSSTKKTQLQLEHLLLDLQMILNGINNYKNPKLTRMLTAKFYMPKKATELKH |
| MDNA109FEAA-C125A- | LQCLEEALKPLEEVLNLAQSKNFHFDPRDVVSNINVFVLELKGSETTFMCEYADE |
| Alb | TATIVEFLNRWITFAQSIISTLTGGGGSGGGGSGGGGSDAHKSEVAHRFKDLGEE |
| (F42A, E62A, L80F, | NFKALVLIAFAQYLQQCPFEDHVKLVNEVTEFAKTCVADESAENCDKSLHTLFGD |
| R81D, L85V, I86V, | KLCTVATLRETYGEMADCCAKQEPERNECFLQHKDDNPNLPRLVRPEVDVMCTAF |
| I92F) | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL |
| SEQ ID NO: 67 | APTSSSTKKTQLQLEHLLLDLQMILNGINNYKNPKLTRMLTAKFYMPKKATELKH |
| hIL2-FEAA-Alb | LQCLEEALKPLEEVLNLAQSKNFHLRPRDLISNINVIVLELKGSETTFMCEYADE |
| (F42A, E62A, L80F, | TATIVEFLNRWITFCQSIISTLTGGGGSGGGGSGGGGSDAHKSEVAHRFKDLGEE |
| R81D, L85V, I86V, | NFKALVLIAFAQYLQQCPFEDHVKLVNEVTEFAKTCVADESAENCDKSLHTLFGD |
| I92F) | KLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKL |
| | DELRDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCIAE |
| | VENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYK |
| | FQNALLVRYTKKVPQVSTPTLVEVSRNLGKVGSKCCKHPEAKRMPCAEDYLSVVL |
| | NQLCVLHEKTPVSDRVTKCCTESLVNRRPCFSALEVDETYVPKEFNAETFTFHAD |
| | ICTLSEKERQIKKQTALVELVKHKPKATKEQLKAVMDDFAAFVEKCCKADDKETC |
| | FAEEGKKLVAASQAALGL |

Further examples of the IL-2 fusion proteins include those listed in the table below:

TABLE 4
Exemplary IL-2 anti-PD-1 and anti-CD3 Fusion Proteins

| SEQ ID NO | |
|---|---|
| (Information) | Amino acid sequence |
| SEQ ID NOs: 173-175 | Gene 1: |
| Anti-mPD1-MDNA109FEAA-C125S | EVQLQESGPGLVKPSQSLSLTCSVTGYSITSSYRWNWIRKFPGNRLEWMGY |
| (KIH) | INSAGISNYNPSLKRRISITRDTSKNQFFLQVNSVTTEDAATYYCARSDNM |
| (F42A, E62A, L80F, R81D, | GTTPFTYWGQGTLVTVSSASTKGPSVFPLAPSSKSTSGGTAALGCLVKDYF |
| L85V, I86V, 192F, C125S) | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGGGGGSGGGGS |
| | GGGGSAPTSSSTKKTQLQLEHLLLDLQMILNGINNYKNPKLTRMLTAKFYM |
| | PKKATELKHLQCLEEALKPLEEVLNLAQSKNFHFDPRDVVSNINVFVLELK |
| | GSETTFMCEYADETATIVEFLNRWITFSQSIISTLT (SEQ ID |
| | NO: 173) |
| | Gene 2: |
| | EVQLQESGPGLVKPSQSLSLTCSVTGYSITSSYRWNWIRKFPGNRLEWMGY |
| | INSAGISNYNPSLKRRISITRDTSKNQFFLQVNSVTTEDAATYYCARSDNM |
| | GTTPFTYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVCTLPPS |
| | RDELTKNQVSLSCAVKGFYPSDIAVEWESNGQPENNYKTTPPVLDSDGSFF |
| | LVSKLTVDKSRWQQGNVFSCSVMHEALHNHYTQKSLSLSPGK (SEQ ID |
| | NO: 174) |
| | Gene 3: |
| | DIVMTQGTLPNPVPSGESVSITCRSSKSLLYSDGKTYLNWYLQRPGQSPQL |
| | LIYWMSTRASGVSDRFSGSGSGTDFTLKISGVEAEDVGIYYCQQGLEFPTF |
| | GGGTKLELKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQG |
| | LSSPVTKSFNRGEC (SEQ ID NO: 175) |
| SEQ ID NOs: 176-178 | Gene 1: |
| Anti-huPD1-MDNA109FEAA- | QVQLVESGGGVVQPGRSLRLDCKASGITFSNSGMHWVRQAPGKGLEWVAVI |
| C125S (KiH) | WYDGSKRYYADSVKGRFTISRDNSKNTLFLQMNSLRAEDTAVYYCATNDDY |
| (F42A, E62A, L80F, R81D, | WGQGTLVTVSSASTKGPSVFPLAPCSRSTSESTAALGCLVKDYFPEPVTVS |
| L85V, I86V, 192F, C125S) | WNSGALTSGVHTFPAVLQSSGLYSLSSVVTVPSSSLGTKTYTCNVDHKPSN |
| | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVYTLPPCRDELTKN |
| | QVSLWCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSAP |
| | TSSSTKKTQLQLEHLLLDLQMILNGINNYKNPKLTRMLTAKFYMPKKATEL |
| | KHLQCLEEALKPLEEVLNLAQSKNFHFDPRDVVSNINVFVLELKGSETTFM |
| | CEYADETATIVEFLNRWITFSQSIISTLT (SEQ ID NO: 176) |
| | Gene 2: |
| | QVQLVESGGGVVQPGRSLRLDCKASGITFSNSGMHWVRQAPGKGLEWVAVI |
| | WYDGSKRYYADSVKGRFTISRDNSKNTLFLQMNSLRAEDTAVYYCATNDDY |
| | WGQGTLVTVSSASTKGPSVFPLAPCSRSTSESTAALGCLVKDYFPEPVTVS |
| | WNSGALTSGVHTFPAVLQSSGLYSLSSVVTVPSSSLGTKTYTCNVDHKPSN |
| | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVCTLPPSRDELTKN |
| | QVSLSCAVKGFYPSDIAVEWESNGQPENNYKTTPPVLDSDGSFFLVSKLTV |
| | DKSRWQQGNVFSCSVMHEALHNHYTQKSLSLSPGK (SEQ ID NO: |
| | 177) |
| | Gene 3: |
| | EIVLTQSPATLSLSPGERATLSCRASQSVSSYLAWYQQKPGQAPRLLIYDA |
| | SNRATGIPARFSGSGSGTDFTLTISSLEPEDFAVYYCQQSSNWPRTFGQGT |
| | KVEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGEC (SEQ ID NO: 178) |
| SEQ ID NOs: 179, 177, and | Gene 1: |
| 178 | QVQLVESGGGVVQPGRSLRLDCKASGITFSNSGMHWVRQAPGKGLEWVAVI |
| Anti-huPD1-MDNA10 9FEAA | WYDGSKRYYADSVKGRFTISRDNSKNTLFLQMNSLRAEDTAVYYCATNDDY |
| (KIH) | WGQGTLVTVSSASTKGPSVFPLAPCSRSTSESTAALGCLVKDYFPEPVTVS |
| (F42A, E62A, L80F, R81D, | WNSGALTSGVHTFPAVLQSSGLYSLSSVVTVPSSSLGTKTYTCNVDHKPSN |
| L85V, I86V, I92F) | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVYTLPPCRDELTKN |
| | QVSLWCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSAP |
| | TSSSTKKTQLQLEHLLLDLQMILNGINNYKNPKLTRMLTAKFYMPKKATEL |
| | KHLQCLEEALKPLEEVLNLAQSKNFHFDPRDVVSNINVFVLELKGSETTFM |
| | CEYADETATIVEFLNRWITFCQSIISTLT (SEQ ID NO: 179) |
| | Gene 2: |
| | QVQLVESGGGVVQPGRSLRLDCKASGITFSNSGMHWVRQAPGKGLEWVAVI |
| | WYDGSKRYYADSVKGRFTISRDNSKNTLFLQMNSLRAEDTAVYYCATNDDY |
| | WGQGTLVTVSSASTKGPSVFPLAPCSRSTSESTAALGCLVKDYFPEPVTVS |
| | WNSGALTSGVHTFPAVLQSSGLYSLSSVVTVPSSSLGTKTYTCNVDHKPSN |
| | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVCTLPPSRDELTKN |
| | QVSLSCAVKGFYPSDIAVEWESNGQPENNYKTTPPVLDSDGSFFLVSKLTV |
| | DKSRWQQGNVFSCSVMHEALHNHYTQKSLSLSPGK |
| | (SEQ ID NO: 177) |
| | Gene 3: |
| | EIVLTQSPATLSLSPGERATLSCRASQSVSSYLAWYQQKPGQAPRLLIYDA |
| | SNRATGIPARFSGSGSGTDFTLTISSLEPEDFAVYYCQQSSNWPRTFGQGT |
| | KVEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGEC (SEQ ID NO: 178) |
| SEQ ID NOs: 180, 174, and | Gene 1: |
| 175 | EVQLQESGPGLVKPSQSLSLTCSVTGYSITSSYRWNWIRKFPGNRLEWMGY |
| Anti-mPD1-MDNA109FEAA (KiH) | INSAGISNYNPSLKRRISITRDTSKNQFFLQVNSVTTEDAATYYCARSDNM |
| (F42A, E62A, L80F, R81D, | GTTPFTYWGQGTLVTVSSASTKGPSVFPLAPSSKSTSGGTAALGCLVKDYF |
| L85V, I86V, I92F) | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGGGGGSGGGGS |
| | GGGGSAPTSSSTKKTQLQLEHLLLDLQMILNGINNYKNPKLTRMLTAKFYM |
| | PKKATELKHLQCLEEALKPLEEVLNLAQSKNFHFDPRDVVSNINVFVLELK |
| | GSETTFMCEYADETATIVEFLNRWITFCQSIISTLT |
| | (SEQ ID NO: 180) |
| | Gene 2: |
| | EVQLQESGPGLVKPSQSLSLTCSVTGYSITSSYRWNWIRKFPGNRLEWMGY |
| | INSAGISNYNPSLKRRISITRDTSKNQFFLQVNSVTTEDAATYYCARSDNM |
| | GTTPFTYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVCTLPPS |
| | RDELTKNQVSLSCAVKGFYPSDIAVEWESNGQPENNYKTTPPVLDSDGSFF |
| | LVSKLTVDKSRWQQGNVFSCSVMHEALHNHYTQKSLSLSPGK |
| | (SEQ ID NO: 174) |
| | Gene 3: |
| | DIVMTQGTLPNPVPSGESVSITCRSSKSLLYSDGKTYLNWYLQRPGQSPQL |
| | LIYWMSTRASGVSDRFSGSGSGTDFTLKISGVEAEDVGIYYCQQGLEFPTF |
| | GGGTKLELKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQG |
| | LSSPVTKSENRGEC (SEQ ID NO: 175) |
| SEQ ID NOs: 181, 177, and | Gene 1: |
| 178 | QVQLVESGGGVVQPGRSLRLDCKASGITFSNSGMHWVRQAPGKGLEWVAVI |
| Anti-huPD1-MDNA109FEAA-T3A- | WYDGSKRYYADSVKGRFTISRDNSKNTLFLQMNSLRAEDTAVYYCATNDDY |
| C125S (1:1 KIH) | WGQGTLVTVSSASTKGPSVFPLAPCSRSTSESTAALGCLVKDYFPEPVTVS |
| (T3A, F42A, E62A, L80F, | WNSGALTSGVHTFPAVLQSSGLYSLSSVVTVPSSSLGTKTYTCNVDHKPSN |
| R81D, L85V, 186V, 192F, | TKVDKRVEPKSCDKTHTCPPCPAPELLGGPSVFLFPPKPKDTLMISRTPEV |
| C125S) | TCVVVDVSHEDPEVKFNWYVDGVEVHNAKTKPREEQYASTYRVVSVLTVLH |
| | QDWLNGKEYKCKVSNKALPAPIEKTISKAKGQPREPQVYTLPPCRDELTKN |
| | QVSLWCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSAP |
| | ASSSTKKTQLQLEHLLLDLQMILNGINNYKNPKLTRMLTAKFYMPKKATEL |
| | KHLQCLEEALKPLEEVLNLAQSKNFHFDPRDVVSNINVFVLELKGSETTFM |
| | CEYADETATIVEFLNRWITFSQSIISTLT (SEQ ID NO: 181) |
| | Gene 2: |
| | QVQLVESGGGVVQPGRSLRLDCKASGITFSNSGMHWVRQAPGKGLEWVAVI |
| | WYDGSKRYYADSVKGRFTISRDNSKNTLFLQMNSLRAEDTAVYYCATNDDY |
| | WGQGTLVTVSSASTKGPSVFPLAPCSRSTSESTAALGCLVKDYFPEPVTVS |
| | WNSGALTSGVHTFPAVLQSSGLYSLSSVVTVPSSSLGTKTYTCNVDHKPSN |
| | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVCTLPPSRDELTKN |
| | QVSLSCAVKGFYPSDIAVEWESNGQPENNYKTTPPVLDSDGSFFLVSKLTV |
| | DKSRWQQGNVFSCSVMHEALHNHYTQKSLSLSPGK (SEQ ID NO: |
| | 177) |
| | Gene 3: |
| | EIVLTQSPATLSLSPGERATLSCRASQSVSSYLAWYQQKPGQAPRLLIYDA |
| | SNRATGIPARFSGSGSGTDFTLTISSLEPEDFAVYYCQQSSNWPRTFGQGT |
| | KVEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGEC (SEQ ID NO: 178) |
| SEQ ID NOs: 182, 177, and | Gene 1: |
| 178 | QVQLVESGGGVVQPGRSLRLDCKASGITFSNSGMHWVRQAPGKGLEWVAVI |
| Anti-huPD1-MDNA109 (KiH) | WYDGSKRYYADSVKGRFTISRDNSKNTLFLQMNSLRAEDTAVYYCATNDDY |
| (L80F, R81D, L85V, 186V, | WGQGTLVTVSSASTKGPSVFPLAPCSRSTSESTAALGCLVKDYFPEPVTVS |
| I92F) | WNSGALTSGVHTFPAVLQSSGLYSLSSVVTVPSSSLGTKTYTCNVDHKPSN |
| | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVYTLPPCRDELTKN |
| | QVSLWCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSAP |
| | TSSSTKKTQLQLEHLLLDLQMILNGINNYKNPKLTRMLTFKFYMPKKATEL |
| | KHLQCLEEELKPLEEVLNLAQSKNFHFDPRDVVSNINVFVLELKGSETTFM |
| | CEYADETATIVEFLNRWITFCQSIISTLT (SEQ ID NO: 182) |
| | Gene 2: |
| | QVQLVESGGGVVQPGRSLRLDCKASGITFSNSGMHWVRQAPGKGLEWVAVI |
| | WYDGSKRYYADSVKGRFTISRDNSKNTLFLQMNSLRAEDTAVYYCATNDDY |
| | WGQGTLVTVSSASTKGPSVFPLAPCSRSTSESTAALGCLVKDYFPEPVTVS |
| | WNSGALTSGVHTFPAVLQSSGLYSLSSVVTVPSSSLGTKTYTCNVDHKPSN |
| | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVCTLPPSRDELTKN |
| | QVSLSCAVKGFYPSDIAVEWESNGQPENNYKTTPPVLDSDGSFFLVSKLTV |
| | DKSRWQQGNVFSCSVMHEALHNHYTQKSLSLSPGK (SEQ ID NO: |
| | 177) |
| | Gene 3: |
| | EIVLTQSPATLSLSPGERATLSCRASQSVSSYLAWYQQKPGQAPRLLIYDA |
| | SNRATGIPARFSGSGSGTDFTLTISSLEPEDFAVYYCQQSSNWPRTFGQGT |
| | KVEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGEC (SEQ ID NO: 178) |
| SEQ ID NOs: 183, 174, and | Gene 1: |
| 175 | EVQLQESGPGLVKPSQSLSLTCSVTGYSITSSYRWNWIRKFPGNRLEWMGY |
| Anti-mPD1-MDNA109 (KiH) | INSAGISNYNPSLKRRISITRDTSKNQFFLQVNSVTTEDAATYYCARSDNM |
| (L80F, R81D, L85V, I86V, | GTTPFTYWGQGTLVTVSSASTKGPSVFPLAPSSKSTSGGTAALGCLVKDYF |
| I92F) | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGGGGGSGGGGS |
| | GGGGSAPTSSSTKKTQLQLEHLLLDLQMILNGINNYKNPKLTRMLTFKFYM |
| | PKKATELKHLQCLEEELKPLEEVLNLAQSKNFHFDPRDVVSNINVFVLELK |
| | GSETTFMCEYADETATIVEFLNRWITFCQSIISTLT (SEQ ID NO: |
| | 183) |
| | Gene 2: |
| | EVQLQESGPGLVKPSQSLSLTCSVTGYSITSSYRWNWIRKFPGNRLEWMGY |
| | INSAGISNYNPSLKRRISITRDTSKNQFFLQVNSVTTEDAATYYCARSDNM |
| | GTTPFTYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVCTLPPS |
| | RDELTKNQVSLSCAVKGFYPSDIAVEWESNGQPENNYKTTPPVLDSDGSFF |
| | LVSKLTVDKSRWQQGNVFSCSVMHEALHNHYTQKSLSLSPGK (SEQ ID |
| | NO: 174) |
| | Gene 3: |
| | DIVMTQGTLPNPVPSGESVSITCRSSKSLLYSDGKTYLNWYLQRPGQSPQL |
| | LIYWMSTRASGVSDRFSGSGSGTDFTLKISGVEAEDVGIYYCQQGLEFPTF |
| | GGGTKLELKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQG |
| | LSSPVTKSFNRGEC (SEQ ID NO: 175) |
| SEQ ID NOS: 184, 174, and | Gene 1: |
| 175 | EVQLQESGPGLVKPSQSLSLTCSVTGYSITSSYRWNWIRKFPGNRLEWMGY |
| Anti-mPD1-H9T (KiH) | INSAGISNYNPSLKRRISITRDTSKNQFFLQVNSVTTEDAATYYCARSDNM |
| (L80F, R81D, L85V, 186V, | GTTPFTYWGQGTLVTVSSASTKGPSVFPLAPSSKSTSGGTAALGCLVKDYF |
| I92F, Q126T) | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGGGGGSGGGGS |
| | GGGGSAPTSSSTKKTQLQLEHLLLDLQMILNGINNYKNPKLTRMLTFKFYM |
| | PKKATELKHLQCLEEELKPLEEVLNLAQSKNFHFDPRDVVSNINVFVLELK |
| | GSETTFMCEYADETATIVEFLNRWITFCTSIISTLT |
| | (SEQ ID NO: 184) |
| | Gene 2: |
| | EVQLQESGPGLVKPSQSLSLTCSVTGYSITSSYRWNWIRKFPGNRLEWMGY |
| | INSAGISNYNPSLKRRISITRDTSKNQFFLQVNSVTTEDAATYYCARSDNM |
| | GTTPFTYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVCTLPPS |
| | RDELTKNQVSLSCAVKGFYPSDIAVEWESNGQPENNYKTTPPVLDSDGSFF |
| | LVSKLTVDKSRWQQGNVFSCSVMHEALHNHYTQKSLSLSPGK (SEQ ID |
| | NO: 174) |
| | Gene 3: |
| | DIVMTQGTLPNPVPSGESVSITCRSSKSLLYSDGKTYLNWYLQRPGQSPQL |
| | LIYWMSTRASGVSDRFSGSGSGTDFTLKISGVEAEDVGIYYCQQGLEFPTF |
| | GGGTKLELKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQG |
| | LSSPVTKSFNRGEC (SEQ ID NO: 175) |
| Mouse AntiCD3-MDNA132 | HC1: |
| | EVQLVESGGGLVQPGKSLKLSCEASGFTFSGYGMHWVRQAPGRGLESVAYI |
| | TSSSINIKYADAVKGRFTVSRDNAKNLLFLQMNILKSEDTAMYYCARFDWD |
| | KNYWGQGTMVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHK |
| | PSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYASTYRVVSVLT |
| | VLHQDWLNGKEYKCKVSNKALPAPIEKTISKAKGQPREPQVYTLPPCRDEL |
| | TKNQVSLWCLVKGFYPSDIAVEWESNGQPENNYKTTPPVLDSDGSFFLYSK |
| | LTVDKSRWQQGNVFSCSVMHEALHNHYTQKSLSLSPGGGGGSGGGGSGGGG |
| | SPGPVPPSTAHRELIEELVNITQNQKAPLCNGSMVWSINLTAGMYCAALES |
| | LINVSGCSAIEKTQRMLSGFCPHKVSAGQFSSLHVTGRKIEVAQFVKDLLL |
| | HLKKLFKEGQFN |
| | (SEQ ID NO: 500) |
| | HC2: |
| | EVQLVESGGGLVQPGKSLKLSCEASGFTFSGYGMHWVRQAPGRGLESVAYI |
| | TSSSINIKYADAVKGRFTVSRDNAKNLLFLQMNILKSEDTAMYYCARFDWD |
| | KNYWGQGTMVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHK |
| | PSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYASTYRVVSVLT |
| | VLHQDWLNGKEYKCKVSNKALPAPIEKTISKAKGQPREPQVCTLPPSRDEL |
| | TKNQVSLSCAVKGFYPSDIAVEWESNGQPENNYKTTPPVLDSDGSFFLVSK |
| | LTVDKSRWQQGNVFSCSVMHEALHNHYTQKSLSLSPGK |
| | (SEQ ID NO: 501) |
| | LC: |
| | DIQMTQSPSSLPASLGDRVTINCQASQDISNYLNWYQQKPGKAPKLLIYYT |
| | NKLADGVPSRFSGSGSGRDSSFTISSLESEDIGSYYCQQYYNYPWTFGPGT |
| | KLEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGEC |
| | (SEQ ID NO: 502) |
| mCD3 IgG-MDNA132^{L39/0111} | Gene 1: |
| (KiH) * | ASTKGPSVFPLAPSSKSTSGGTAALGCLVKDYFPEPVTVSWNSGALTSGVH |
| (*partial sequence) | TFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHED |
| | PEVKFNWYVDGVEVHNAKTKPREEQYASTYRVVSVLTVLHQDWLNGKEYKC |
| | KVSNKALPAPIEKTISKAKGQPREPQVYTLPPCRDELTKNQVSLWCLVKGF |
| | YPSDIAVEWESNGQPENNYKTTPPVLDSDGSFFLYSKLTVDKSRWQQGNVF |
| | SCSVMHEALHNHYTQKSLSLSPGGGGGSGGGGSGGGGSPGPVPPSTAHREL |
| | IEELVNITQNQKAPLCNGSMVWSINLTAGMYCAALESLINVSGCSAIEKTQ |
| | RMLSGFCPHKVSAGQFSSLHVTGRKIEVAQFVKDLLLHLKKLFKEGQFN |
| | (SEQ ID NO: 503) |
| | Gene 2: |
| | ASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHED |
| | PEVKFNWYVDGVEVHNAKTKPREEQYASTYRVVSVLTVLHQDWLNGKEYKC |
| | KVSNKALPAPIEKTISKAKGQPREPQVCTLPPSRDELTKNQVSLSCAVKGF |
| | YPSDIAVEWESNGQPENNYKTTPPVLDSDGSFFLVSKLTVDKSRWQQGNVF |
| | SCSVMHEALHNHYTQKSLSLSPGK (SEQ ID NO: 504) |
| | Gene 3: |
| | RTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSF |
| | NRGEC (SEQ ID NO: 505) |
| huCD3 IgG-MDNA132^{L39/0111} | Gene 1: |
| (KiH) * | ASTKGPSVFPLAPSSKSTSGGTAALGCLVKDYFPEPVTVSWNSGALTSGVH |
| (*partial sequence) | TFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHED |
| | PEVKFNWYVDGVEVHNAKTKPREEQYASTYRVVSVLTVLHQDWLNGKEYKC |
| | KVSNKALPAPIEKTISKAKGQPREPQVYTLPPCRDELTKNQVSLWCLVKGF |
| | YPSDIAVEWESNGQPENNYKTTPPVLDSDGSFFLYSKLTVDKSRWQQGNVF |
| | SCSVMHEALHNHYTQKSLSLSPGGGGGSGGGGSGGGGSPGPVPPSTAHREL |
| | IEELVNITQNQKAPLCNGSMVWSINLTAGMYCAALESLINVSGCSAIEKTQ |
| | RMLSGFCPHKVSAGQFSSLHVTGRKIEVAQFVKDLLLHLKKLFKEGQFN |
| | (SEQ ID NO: 506) |
| | Gene 2: |
| | ASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHED |
| | PEVKFNWYVDGVEVHNAKTKPREEQYASTYRVVSVLTVLHQDWLNGKEYKC |
| | KVSNKALPAPIEKTISKAKGQPREPQVCTLPPSRDELTKNQVSLSCAVKGF |
| | YPSDIAVEWESNGQPENNYKTTPPVLDSDGSFFLVSKLTVDKSRWQQGNVF |
| | SCSVMHEALHNHYTQKSLSLSPGK (SEQ ID NO: 507) |
| | Gene 3: |
| | RTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSF |
| | NRGEC (SEQ ID NO: 508) |

### C. IL-4 and IL-13 Receptor Targeted Cargo Moieties

The subject treatment methods provided herein utilize an IL-4 receptor or IL-13 receptor targeted moiety, comprising an IL-4s or IL-13s disclosed linked to a cargo moiety that functions to reduce or inhibit cancer stem cell growth, or kill cancer cells and/or cancer stem cells. Without being bound by any particular theory of operation, it is believed that IL-4s linked to such a cargo moiety are capable of targeting the cargo moiety to cancer cells that overexpress IL-4R, thereby allowing the linked cargo moiety to act locally in the tumor microenvironment to promote anti-tumor activity. Similarly, and without being bound by any particular theory of operation, it is believed that IL-13s linked to such a cargo moiety are capable of targeting the cargo moiety to cancer cells that overexpress IL-13 receptors, thereby allowing the linked cargo moiety to act locally in the tumor microenvironment to promote anti-tumor activity. Thus, the IL-4 and IL-13 function as a targeting moiety for the cargo moiety.

Any IL-13 sequence or variant thereof can be in the IL-4R/IL-13 receptors targeted moiety as described herein. In some embodiments, the IL-13 polypeptide sequence is 90% identical to any one the sequences provided herein. In some embodiments, the IL-13 polypeptide sequence is 95% identical to any one the sequences provided herein. In some embodiments, the IL-13 polypeptide sequence is 98% identical to any one the sequences provided herein. In some embodiments, the IL-13 polypeptide sequence is 99% identical to any one the sequences provided herein. Exemplary sequences of IL-13 are provided in Tables 5, 6, and 7 below.

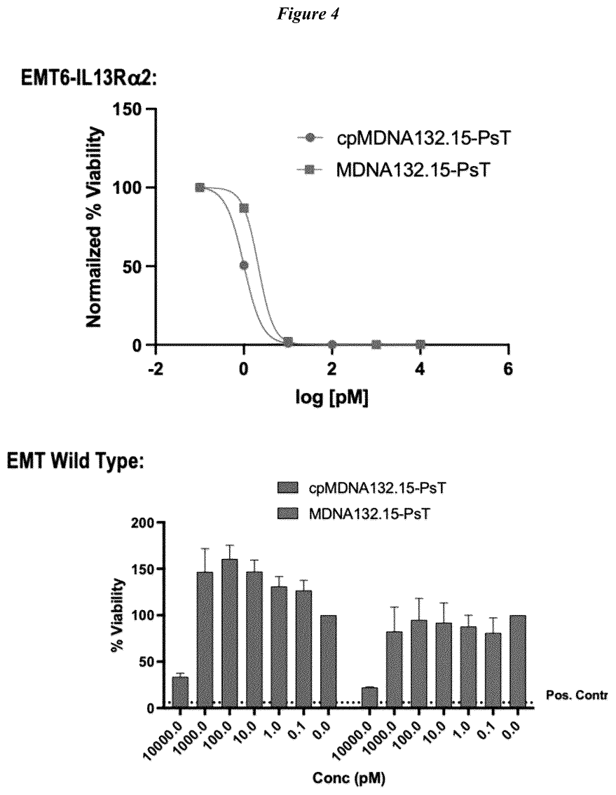

In some embodiments an IL-13 peptide comprises one or more of the amino acids substitutions: (1) L10F, L10I, L10V, L10A, L10D, L10T, L10H; (2) R11S, R11N, R11H, R11L, R111; (3) 114L, 114F, 114V, 114M; (4) V18L, V18F, V181; (5) E12A, (6) R65D, (7) R86K, R86T, R86M; (8) D87E, D87K, D87R, D87G, D87S; (9) T881, T88K, T88R; (10) K89R, K89T, K89M; (11) L101F, L1011, L101Y, L101H, L101N; (12) K104R, K104T, K104M; (13) K105T, K105A, K105R, K105E; (14) F107L, F1071, F107V, F107M; (15) R108K, R108T, R108M; and (16) E15R, which substitutions cause an altered affinity for one or both of IL-13Rα1 and IL-13Rα2. In other embodiments, modified residues are at two or more, three or more, four or more, five or more, and not more than 14 amino acids within the combined set of contact residues defined above. As described in International Patent Publication WO 2013/112871, the disclosure of which is incorporated by reference herein in its entirety. In some embodiments, amino acid substitutions include without limitation those provided in FIG. 4.

Sets of modifications may include the following specific changes: (1) L10H; L10A; (2) R11L; (4) V181; (7) R86M; R86K; R86T; (8) D87K; D87G; (9) T88R, T88S; T88K; (10) K89R; (11) L101N; (12) K104R; (13) K105A; K105E; (14) R108K; (15) E15R. In some embodiments, the modification includes any one of the recited specific changes. In some embodiments, the modification includes L10H. In some embodiments, the modification includes L10A. In some embodiments, the modification includes R11L. In some embodiments, the modification includes E15R. In some embodiments, the modification includes V181. In some embodiments, the modification includes R86M. In some embodiments, the modification includes R86K. In some embodiments, the modification includes R86T. In some embodiments, the modification includes D87K. In some embodiments, the modification includes D87G. In some embodiments, the modification includes T88R. In some embodiments, the modification includes T88S. In some embodiments, the modification includes T88K. In some embodiments, the modification includes K89R. In some embodiments, the modification includes L101N. In some embodiments, the modification includes K104R. In some embodiments, the modification includes K105A. In some embodiments, the modification includes K105E. In some embodiments, the modification includes R108K. In some embodiments, the polypeptide comprising the one or more modifications is linked to an IL-2 or IL-2 mutein as described herein. In some embodiments, the IL-2 mutein incudes any one of SEQ ID NO:1 through SEQ ID NO:20.

Specific sets of modifications that provide for greater selectivity in binding to IL-13Rα2 versus IL-13Rα1 relative to a native IL-13 sequence may include, without limitation:
[10D, R111, V181, R86K, D87K, k89R, R108K](for example, C2)
[L10A, R86T, D87G, T88K, K89R, L101N, K104R, K105A, R108K](for example, C3)
[L10V, K89R, L101N, K105E, R108T](for example, C4)
[R11S, 114M, T88S, L101N, K105A, R108K](for example, C7)
[L10H, R11L, V181, R86K, D87E, K89R, L101N, K105T, R108K](C9)
[L10H, R86T, D87G, T88R, R108K](C11)
[L10H, E15R, R86T, D87G, T88R, R108K](MDNA132+E15R)
[L10A, V18F, R86K, D87K, K89R, L1011, K104R, R108K](D7)
[L10T/D; R111; V181; R86K; D87K/G; T88S; K89R; L101Y; K104R; K105T; R108K]
[L10A/V; R86T; D87G; T88K; K89R; L101N; K104R; K105A/E; R108K/T]

Specific sets of modifications that provide for greater selectivity in binding to IL-13Rα1 v IL-13Rα2 relative to a native IL-13 sequence may include, without limitation:
[L10V, V181, D87S, D88S, L101F, K104R, K105T];
[R11S, V181, R86K, D87G, T88S, K89M, 101Y, K104R, K105T];
[L10V, V181, D87S, T88S, L101F, K104R, K105T];
[L10V/l; D87S; T88S; K89R; L101H/F; K104R; K105T];
[101; V181; R86T; D87G; T88S; K89R; L101Y/H; K104R; K105A];
[L10V; V181; D87S; T88S; L101F; K104R; K105T];
[V181, R86T, D87G, T88S, 101Y, K104R, K105A];
[R111, V181, R86K, D87G, T88S, L101H, K104R, K105A, F107M];
which substitutions are optionally combined with the substitutions [E12A/G/S, R65D/E];
[L10V, V181, D87S, T88S, L101F, K104R, K105T, and R39 polymorphism];
[L10V, V181, D87S, T88S, L101F, K104R, K105T, and Q111 polymorphism];
[L10V, V181, D87S, T88S, L101F, K104R, K105T, and R39 and Q111 polymorphism].
[E15R]
[L10V, V181, D87S, D88S, L101F, K104R, K105T, E15R];
[R11S, V181, R86K, D87G, T88S, K89M, L101Y, K104R, K105T, E15R];
[10V, V181, D87S, T88S, L101F, K104R, K105T, E15R];
[L10V/l; D87S; T88S; K89R; L101H/F; K104R; K105T, E15R];
[L101; V181; R86T; D87G; T88S; K89R; L101Y/H; K104R; K105A, E15R];
[10V; V181; D87S; T88S; [L101F; K104R; K105T, E15R];
[V181, R86T, D87G, T88S, L101Y, K104R, K105A, E15R];
[R111, V181, R86K, D87G, T88S, L101H, K104R, K105A, F107M, E15R];
which substitutions are optionally combined with the substitutions [E12A/G/S, R65D/E] and/or E15R];
[10V, E15R, V181, D87S, T88S, L101F, K104R, K105T, and R39 polymorphism];
[10V, E15R, V181, D87S, T88S, L101F, K104R, K105T, and Q111 polymorphism]; and
[10V, E15R,V181, D87S, T88S, L101F, K104R, K105T, and R39 and Q111 polymorphism].

Exemplary IL-13 sequences are provided below.

TABLE 5
Exemplary IL-13 Muteins

| SEQ ID NO | |
|---|---|
| (Information) | Amino acid sequence |
| SEQ ID NO: 73 | PGPVPPSTALRELIEELVNITQNQKAPLCNGSMVWSINLTAGMY |
| IL-13-WT-L39/Q111 | CAALESLINVSGCSAIEKTQRMLSGFCPHKVSAGQFSSLHVRDT |
| | KIEVAQFVKDLLLHLKKLFREGQFN |
| SEQ ID NO: 74 | PGPVPPSTAVRALIEELINITQNQKAPLCNGSMVWSINRTAGMY |
| MDNA413-R39/Q111 | CAALESLINVSGCSAIEKTQDMLSGFCPHKVSAGQFSSLHVRSS |
| (L10V, E12A, V18I, R65D, D87S, | KIEVAQFVKDLLFHLRTLFREGQFN |
| T88S, L101F, K104R, K105T) | |
| SEQ ID NO: 75 | PGPVPPSTAIRELIEELINITQNQKAPLCNGSMVWSINLTAGMY |
| *L39/Q111 | CAALESLINVSGCSAIEKTQRMLSGFCPHKVSAGQFSSLHVRGS |
| (L10I, V18I, D87G, T88S, L101H, | KIEVAQFVKDLLHHLRALFREGQFN |
| K104R, K105A) | |
| SEQ ID NO: 76 | PGPVPPSTALIELIEELINITQNQKAPLCNGSMVWSINLTAGIY |
| A5-M43I-L39/Q111 | CAALESLINVSGCSAIEKTQRMLSGFCPHKVSAGQFSSLHVKGS |
| (R11I, V18I, M43I, R86K, D87G, | KIEVAQFVKDLLHHLRALMREGQFN |
| T88S, L101H, K104R, K105A, | |
| F107M) | |
| SEQ ID NO: 77 | PGPVPPSTAIRELIEELLNITQNQKAPLCNGSMVWSINLTAGMY |
| A6-L39/Q111 | CAALESLINVSGCSAIEKTQRMLSGFCPHKVSAGQFSSLHVMKS |
| (L10I, V18L, R86M, D87K, T88S, | KIEVAQFVKDLLHHLRALFREGQFN |
| L101H, K104R, K105A) | |
| SEQ ID NO: 78 | PGPVPPSTAIRELIEELINITQNQKAPLCNGSMVWSINLTAGMY |
| A8-L39/Q111 | CAALESLINVSGCSAIEKTQRMLSGFCPHKVSAGQFSSLHVRSS |
| (L10I, V18I, D87S, T88S, K89R, | RIEVAQFVKDLLHHLRTLFREGQFN |
| L101H, K104R, K105T) | |
| SEQ ID NO: 79 | PGPVPPSTALRELIEELINITQNEKAPLCNGSMVWSINLTAGIY |
| *L39/Q111 | CAALESLINVSGCSAIEKTQRMLSGFCPHKVSAGQFSSLHVTGS |
| (V18I, Q24E, M43I, R86T, D87G, | KIEVAQFVKDLLYHLRALFREGQFN |
| T88S, L101Y, K104R, K105A) | |
| SEQ ID NO: 80 | PGPVPPSTALSELIEELINITQNQKAPLCNGSMVWSINPTAGMY |
| *Q111 | CAALESLINVSGCSAIEKTQRMLSGFCPHKVAAGQFSSLHDKGS |
| (R11S, V18I, L39P, S76A, V85D, | MIEVAQFVKDLLYHLRTLFREGQFN |
| R86K, D87G, T88S, K89M, L101Y, | |
| K104R, K105T) | |
| SEQ ID NO: 81 | PGPVPPSTATRELIEELINITQNQKAPLCNGSMVWSINLTADMY |
| *L39/Q111 | CAALESLINVSGCSAIEKTQRMLSGFCPHKVSVGQFSSLHVRGS |
| (L10T, V18I, G42D, A77V, D87G, | KIEVAQFVKDLLYHLRTLFREGQFN |
| T88S, L101Y, K104R, K105T) | |
| SEQ ID NO: 82 | PGPVPPSTADIELIAELINITQNQKAPLCNGSMVWSINLTADMY |
| *L39/Q111 | CAALESLINVSGCSAIEKTQRMLSGFCPHKVSAGQFSSLHVKKT |
| (L10D, R11I, E15A, V18I, G42D, | RIEVAQFVKDLLLHLKKLFKEGQFN |
| R86K, D87K, K89R, R108K) | |
| SEQ ID NO: 83 | PGPVPPSTAARELIEELVNITQNQKAPLCNGSMVWSINLTAGMY |
| *L39/Q111 | CAALESLINVSGCSAIEKTQRMLSGFCPHKVSAGQLSSLHVTGK |
| (L10A, F80L, R86T, D87G, T88K, | RIEVAQFVKDLLNHLRALFKEGQFN |
| L101N, K104R, K105A, R108K) | |
| SEQ ID NO: 84 | PGPVPPSTAVRELIEELVNITQNQKAPLCNGSMVWSINLTAGMY |
| C4-L39/Q111 | CAALESLINVSGCSAIEKTQRMLSGFCPHKVSAGQFSSLHVRDT |
| (L10V, K89R, L101N, K105E, | RIEVAQFVKDLLNHLKELFTEGQFN |
| R108T) | |
| SEQ ID NO: 85 | PGPVPPSTALSELMEELVNITQNQKAPLCNGSMVWSINLTAGMY |
| C7-L39/Q111 | CAALESLINVSGCSAIEKTQRMLSGFCPHKVSAGQFSSLHVRDS |
| (R11S, I14M, T88S, L101N, | KIEVAQFVKDLLNHLKALFKEGQFN |
| K105A, R108K) | |
| SEQ ID NO: 86 | GPVPPSTAFRELIEELVNITQNQKAPLCNGSMVWSINLTAGMYC |
| *L39/Q111 | AALESLINVSGCSAIEKTQRMLSGFCPHKVSPGQFSSLHVTNSR |
| (dP1, L10F, A77P, R86T, D87N, | IEVAQFVKDLLNHLKALFKEGQYN |
| T88S, K89R, L101N, K105A, | |
| R108K, F112Y) | |
| SEQ ID NO: 87 | GPVPPSTAHLELIEELINITQNQKAPLCNGSMVWSINLTAGMYC |
| *L39/Q111 | AALESLINVSGCSAIEKTQRMLSGFCPHKVSAGQFSSLHVKETR |
| (dP1, L10H, R11L, V18I, R86K, | IEVAQFVKDLLNHLKTLFKEGQFN |
| D87E, K89R, L101N, K105T, | |
| R108K) | |
| SEQ ID NO: 88 | PGPVPPSTAHLELIEELINITQNQKAPLCNGSMVWSINPTAGMY |
| *Q111 | CAALESLINVSGCSAIEKTQRMLSGFCPHKVSAGQFSSLHVMDT |
| (L10H, R11L, V18I, L39P, R86M, | RIEVAQFVKDLLLHLKKLFKEGQFN |
| K89R, R108K) | |
| SEQ ID NO: 89 | PGPVPPSTAHRELIEELVNITQNQKAPLCNGSMVWSINLTAGMY |
| MDNA132-L39/Q111 | CAALESLINVSGCSAIEKTQRMLSGFCPHKVSAGQFSSLHVTGR |
| (L10H, R86T, D87G, T88R, R108K) | KIEVAQFVKDLLLHLKKLFKEGQFN |
| SEQ ID NO: 90 | PGPVPPSTAHRELIEELVNITQNQKAPLCNGSMVWRINRTAGMY |
| *R39/Q111 | CAALESLINVSGCSAIEKTQRMLSGFCPHKVSAGQFSSLHVMDS |
| (L10H, S36R, L39R, R86M, T88S, | RIEVAQFVKDLLNHLRALFKEGQFN |
| K89R, L101N, K104R, K105A, | |
| R108K) | |
| SEQ ID NO: 91 | PGPVPPSTAARELIEELFNITQNQKAPLCNGSMVWSINLTAGMY |
| *L39/Q111 | CAALESLINVSGCSAIEKTKRMLSGFCPHKVSAGQFPSLHVKKT |
| (L10A, V18F, Q64K, S81P, R87K, | RIEVAQFVKDLLIHLRKLFKEGQFN |
| D87K, K89R, L101I, K104R, | |
| R108K) | |
| SEQ ID NO: 92 | PGPVPPSTALIELIEELINITQNQKAPLCNGSMVWSINLTAGMY |
| A5-L39/Q111 | CAALESLINVSGCSAIEKTQRMLSGFCPHKVSAGQFSSLHVKGS |
| (R11I, V18I, R86K, D87G, T88S, | KIEVAQFVKDLLHHLRALMREGQFN |
| L101H, K104R, K105A, F107M) | |
| SEQ ID NO: 93 | PGPVPPSTAIRELIEELLNITQNQKAPLCNGSMVWSINLTAGMY |
| A6-L39/Q111 | CAALESLINVSGCSAIEKTQRMLSGFCPHKVSAGQFSSLHVMKS |
| (L10I, V18L, R86M, D87K, T88S, | KIEVAQFVKDLLHHLRALFREGQFN |
| L101H, K104R, K105A) | |
| SEQ ID NO: 94 | PGPVPPSTAIRELIEELINITQNQKAPLCNGSMVWSINLTAGMY |
| A7-L39/Q111 | CAALESLINVSGCSAIEKTQRMLSGFCPHKVSAGQFSSLHVRGS |
| (L10I, V18I, D87G, T88S, L101H, | KIEVAQFVKDLLHHLRALFREGQFN |
| K104R, K105A) | |
| SEQ ID NO: 95 | PGPVPPSTAIRELIEELINITQNQKAPLCNGSMVWSINLTAGMY |
| A8-L39/Q111 | CAALESLINVSGCSAIEKTQRMLSGFCPHKVSAGQFSSLHVRSS |
| (L10I, V18I, D87S, T88S, K89R, | RIEVAQFVKDLLHHLRTLFREGQFN |
| L101H, K104R, K105T) | |
| SEQ ID NO: 96 | PGPVPPSTALRELIEELINITQNQKAPLCNGSMVWSINLTAGMY |
| B2-L39/Q111 | CAALESLINVSGCSAIEKTQRMLSGFCPHKVSAGQFSSLHVTGS |
| (V18I, R86T, D87G, T88S, L101Y, | KIEVAQFVKDLLYHLRALFREGQFN |
| K104R, K105A) | |
| SEQ ID NO: 97 | PGPVPPSTALSELIEELINITQNQKAPLCNGSMVWSINLTAGMY |
| B4-L39/Q111 | CAALESLINVSGCSAIEKTQRMLSGFCPHKVSAGQFSSLHVKGS |
| (R11S, V18I, R86K, D87G, T88S, | MIEVAQFVKDLLYHLRTLFREGQFN |
| K89M, L101Y, K104R, K105T) | |
| SEQ ID NO: 98 | PGPVPPSTATRELIEELINITQNQKAPLCNGSMVWSINLTAGMY |
| B6-L39/Q111 | CAALESLINVSGCSAIEKTQRMLSGFCPHKVSAGQFSSLHVRGS |
| (L10T, V18I, D87G, T88S, K89K, | KIEVAQFVKDLLYHLRTLFREGQFN |
| L101Y, K104R, K105T) | |
| SEQ ID NO: 99 | PGPVPPSTADIELIEELINITQNQKAPLCNGSMVWSINLTAGMY |
| C2-L39/Q111 | CAALESLINVSGCSAIEKTQRMLSGFCPHKVSAGQFSSLHVKKT |
| (L10D, R11I, V18I, R86K, D87K, | RIEVAQFVKDLLLHLKKLFKEGQFN |
| K89R, R108K) | |
| SEQ ID NO: 100 | PGPVPPSTAARELIEELVNITQNQKAPLCNGSMVWSINLTAGMY |
| C3-L39/Q111 | CAALESLINVSGCSAIEKTQRMLSGFCPHKVSAGQFSSLHVTGK |
| (L10A, R86T, D87G, T88K, K89R, | RIEVAQFVKDLLNHLRALFKEGQFN |
| L101N, K104R, K105A, R108K) | |
| SEQ ID NO: 101 | PGPVPPSTAVRELIEELVNITQNQKAPLCNGSMVWSINLTAGMY |
| C4-L39/Q111 | CAALESLINVSGCSAIEKTQRMLSGFCPHKVSAGQFSSLHVRDT |
| (L10V, K89R, L101N, K105E, | RIEVAQFVKDLLNHLKELFTEGQFN |
| R108T) | |
| SEQ ID NO: 102 | PGPVPPSTALSELMEELVNITQNQKAPLCNGSMVWSINLTAGMY |
| C7-L39/Q111 | CAALESLINVSGCSAIEKTQRMLSGFCPHKVSAGQFSSLHVRDS |
| (R11S, I14M, T88S, L101N, | KIEVAQFVKDLLNHLKALFKEGQFN |
| K105A, R108K) | |
| SEQ ID NO: 103 | PGPVPPSTAHLELIEELINITQNQKAPLCNGSMVWSINLTAGMY |
| | CAALESLINVSGCSAIEKTQRMLSGFCPHKVSAGQFSSLHVKET |
| C9-L39/Q111 | RIEVAQFVKDLLNHLKTLFKEGQFN |
| (L10H, R11L, V18I, R86K, D87E, | |
| K89R, L101N, K105T, R108K) | |
| SEQ ID NO: 104 | PGPVPPSTAHLELIEELINITQNQKAPLCNGSMVWSINLTAGMY |
| C10-L39/Q111 | CAALESLINVSGCSAIEKTQRMLSGFCPHKVSAGQFSSLHVMDT |
| (L10H, R11L, V18I, R86M, K89R, | RIEVAQFVKDLLLHLKKLFKEGQFN |
| R108K) | |
| SEQ ID NO: 105 | PGPVPPSTAHRELIEELVNITQNQKAPLCNGSMVWSINLTAGMY |
| MDNA132-L39/Q111 | CAALESLINVSGCSAIEKTQRMLSGFCPHKVSAGQFSSLHVTGR |
| (L10H, R86T, D87G, T88R, R108K) | KIEVAQFVKDLLLHLKKLFKEGQFN |
| SEQ ID NO: 106 | PGPVPPSTAHRELIRELVNITQNQKAPLCNGSMVWSINLTAGMY |
| MDNA132.15-L39/Q111 | CAALESLINVSGCSAIEKTQRMLSGFCPHKVSAGQFSSLHVTGR |
| (L10H, E15R, R86T, D87G, T88R, | KIEVAQFVKDLLLHLKKLFKEGQFN |
| R108K, Q111) | |
| (Also referred to as MDNA213) | |
| SEQ ID NO: 107 | PGPVPPSTAHRELIEELVNITQNQKAPLCNGSMVWSINLTAGMY |
| MDNA132-L39/R111 | CAALESLINVSGCSAIEKTQRMLSGFCPHKVSAGQFSSLHVTGR |
| (L10H, R86T, D87G, T88R, R108K) | KIEVAQFVKDLLLHLKKLFKEGREN |
| SEQ ID NO: 108 | PGPVPPSTAHRELIEELVNITQNQKAPLCNGSMVWSINLTAGMY |
| C12-L39/Q111 | CAALESLINVSGCSAIEKTQRMLSGFCPHKVSAGQFSSLHVMDS |
| (L10H, R86M, T88S, K89R, L101N, | RIEVAQFVKDLLNHLRALFKEGQFN |
| K104R, K105A, R108K) | |
| SEQ ID NO: 109 | PGPVPPSTAARELIEELFNITQNQKAPLCNGSMVWSINLTAGMY |
| D7-L39/Q111 | CAALESLINVSGCSAIEKTQRMLSGFCPHKVSAGQFSSLHVKKT |
| (L10A, V18F, R86K, D87K, K89R, | RIEVAQFVKDLLIHLRKLFKEGQFN |
| L101I, K104R, R108K) | |
| SEQ ID NO: 110 | PGPVPPSTAVRALIEELINITQNQKAPLCNGSMVWSINLTAGMY |
| MDNA413-L39/Q111 | CAALESLINVSGCSAIEKTQDMLSGFCPHKVSAGQFSSLHVRSS |
| (L10V, E12A, V18I, R65D, D87S, | KIEVAQFVKDLLFHLRTLFREGQFN |
| T88S, L101F, K104R, K105T) | |
| SEQ ID NO: 111 | PGPVPPSTAHRELIEELVNITQNQKAPLCNGSMVWSINRTAGMY |
| MDNA132-R39/Q111 | CAALESLINVSGCSAIEKTQRMLSGFCPHKVSAGQFSSLHVTGR |
| (L10H, R86T, D87G, T88R, R108K) | KIEVAQFVKDLLLHLKKLFKEGQFN |
| SEQ ID NO: 112 | PGPVPPSTAVRALIEELINITQNQKAPLCNGSMVWSINRTAGMY |
| MDNA413-R39/Q111 | CAALESLINVSGCSAIEKTQDMLSGFCPHKVSAGQFSSLHVRSS |
| (L10V, E12A, V18I, R65D, D87S, | KIEVAQFVKDLLFHLRTLFREGQFN |
| T88S, L101F, K104R, K105T) | |
| SEQ ID NO: 113 | MPGPVPPSTALRELIEELVNITQNQKAPLCNGSMVWSINLTAGM |
| wild-type IL-13 including an | YCAALESLINVSGCSAIEKTQRMLSGFCPHKVSAGQFSSLHVRD |
| additional methionine at the N- | TKIEVAQFVKDLLLHLKKLFREGQFN |
| terminus | |
| SEQ ID NO: 114 | MYCAALESLINVSGCSAIEKTQRMLSGFCPHKVSAGQFSSLHVR |
| circularly permuted IL-13- | DTKIEVAQFVKDLLLHLKKLFREGQFNGGSGPGPVPPSTALREL |
| L39/Q111 | IEELVNITQNQKAPLCNGSMVWSINLTAG |
| SEQ ID NO: 115 | MYCAALESLINVSGCSAIEKTQRMLSGFCPHKVSAGQFSSLHVR |
| circularly permuted IL-13- | DTKIEVAQFVKDLLLHLKKLFREGQFNGGSGMPGPVPPSTALRE |
| L39/Q111-withM | LIEELVNITQNQKAPLCNGSMVWSINLTAG |
| SEQ ID NO: 116 | MYCAALESLINVSGCSAIEKTQDMLSGFCPHKVSAGQFSSLHVR |
| circularly permuted MDNA413- | SSKIEVAQFVKDLLFHLRTLFREGQFNGGSGPGPVPPSTAVRAL |
| L39/Q111 | IEELINITQNQKAPLCNGSMVWSINLTAG |
| (L10V, E12A, V18I, R65D, D87S, | |
| T88S, L101F, K104R, K105T) | |
| SEQ ID NO: 117 | MYCAALESLINVSGCSAIEKTQDMLSGFCPHKVSAGQFSSLHVR |
| circularly permuted MDNA413- | SSKIEVAQFVKDLLFHLRTLFREGQFNGGSGMPGPVPPSTAVRA |
| L39/Q111-withM | LIEELINITQNQKAPLCNGSMVWSINLTAG |
| (L10V, E12A, V18I, R65D, D87S, | |
| T88S, L101F, K104R, K105T) | |
| SEQ ID NO: 118 | PGPVPPSTAVRELIEELINITQNQKAPLCNGSMVWSINRTAGMY |
| A11 | CAALESLINVSGCSAIEKTQRMLSGFCPHKVSAGQFSSLHVRSS |
| (L10V, V18I, D87S, T88S, L101F, | KIEVAQFVKDLLFHLRTLFREGQFN |
| K104R, K105T) | |
| SEQ ID NO: 159 | MTAGMYCAALESLINVSGCSAIEKTQRMLSGFCPHKVSAGQFSS |
| cpMDNA132.15-L39/Q111 | LHVTGRKIEVAQFVKDLLLHLKKLFKEGQFNGGNGGPGPVPPST |
| (L10H, E15R, R86T, D87G, T88R, | AHRELIRELVNITQNQKAPLCNGSMVWSINL |
| R108K) | |
| SEQ ID NO: 160 | MTAGMYCAALESLINVSGCSAIEKTQRMLSGFCPHKVSAGQFSS |
| cpMDNA132.15-L39/R111 | LHVTGRKIEVAQFVKDLLLHLKKLFKEGRENGGNGGPGPVPPST |
| (L10H, E15R, R86T, D87G, T88R, | AHRELIRELVNITQNQKAPLCNGSMVWSINL |
| R108K) | |
| SEQ ID NO: 139 | HHHHHHENLYFQGPGPVPPSTAVRALIEELINITQNQKAPLQNG |
| HisTag-MDNA413-R39/Q111 | SMVWSINRTAGMYCAALESLINVSGCSAIEKTQDMLSGFCPHKV |
| (L10V, E12A, V18I, L39R, R65D, | SAGQFSSLHVRSSKIEVAQFVKDLLFHLRTLFREGQFN |
| D87S, T88S, L101F, K104R, | |
| K105T) | |

Any IL-4 sequence or variant thereof can be used as an IL-4R targeting moiety, including those as described herein. Exemplary IL-4 polypeptide sequences and variants thereof are provided in Table 6.

In some embodiments, the IL-4 component comprises the following substitutions: R121K, Y124F, S125R, as compared to wild-type IL-4. In some embodiments, the IL-4 component comprises the following substitutions: K117R, T118V, R121Q, D122S, Y124W, S125F, S128G, S129A, as compared to wild-type IL-4.

Table of IL-4 sequences is provided below.

TABLE 6
Exemplary IL-4 Muteins

| SEQ ID NO: | |
|---|---|
| (Information) | Amino acid sequence |
| SEQ ID NO: 1050 | MGLTSQLLPPLFFLLACAGNFVHGHKCDITLQEIIKTLNSLTEQK |
| (IL-4 wildtype with signal | TLCTELTVTDIFAASKNTTEKETFCRAATVLRQFYSHHEKDTRCL |
| peptide) | GATAQQFHRHKQLIRFLKRLDRNLWGLAGLNSCPVKEANQSTLEN |
| | FLERLKTIMREKYSKCSS |
| SEQ ID NO: 1051 | MHKCDITLQEIIKTLNSLTEQKTLCTELTVTDIFAASKDTTEKET |
| IL-4 including an additional | FCRAATVLRQFYSHHEKDTRCLGATAQQFHRHKQLIRFLKRLDRN |
| methionine at the N- | LWGLAGLNSCPVKEANQSTLENFLERLKTIMREKYSKCSS |
| terminus″ starting | |
| SEQ ID NO: 1052 | KCDITLQEIIKTLNSLTEQKTLCTELTVTDIFAASKNTTEKETFC |
| KFR | RAATVLRQFYSHHEKDTRCLGATAQQFHRHKQLIRFLKRLDRNLW |
| | GLAGLNSCPVKEANQSTLENFLERLKTIMKEKFRKCSS |
| SEQ ID NO: 1053 | MDTTEKETFCRAATVLRQFYSHHEKDTRCLGATAQQFHRHKQLIR |
| Circularly permuted RGA | FLKRLDRNLWGLAGLNSCPVKEANQSTLENFLERLRVIMQSKWFK |
| | CGAGGNGGHKCDITLQEIIKTLNSLTEQKTLCTELTVTDIFAAS |
| SEQ ID NO: 1054 | MDTTEKETFCRAATVLRQFYSHHEKDTRCLGATAQQFHRHKQLIR |
| circularly permuted wild- | FLKRLDRNLWGLAGLNSCPVKEANQSTLENFLERLKTIMREKYSK |
| type IL-4 | CSSGGNGGHKCDITLQEIIKTLNSLTEQKTLCTELTVTDIFAAS |
| SEQ ID NO: 1055 | MDTTEKETFCRAATVLRQFYSHHEKDTRCLGATAQQFHRHKQLIR |
| circularly permuted “KFR” | FLKRLDRNLWGLAGLNSCPVKEANQSTLENFLERLKTIMKEKFRK |
| IL-4 variant | CSSGGNGGHKCDITLQEIIKTLNSLTEQKTLCTELTVTDIFAAS |
| SEQ ID NO: 1056 | MDTTEKETFCRAATVLRQFYSHHEKDTRCLGATAQQFHRHKQLIR |
| circularly permuted “KF” IL- | FLKRLDRNLWGLAGLNSCPVKEANQSTLENFLERLKTIMKEKFKC |
| 4 variant | SSGGNGGHKCDITLQEIIKTLNSLTEQKTLCTELTVTDIFAAS |
| SEQ ID NO: 1057 | MDTTEKETFCRAATVLRQFYSHHEKDTRCLGATAQQFHRHKQLIR |
| MDNA55 | FLKLRDRNLWGLAGLNSCPVKEANQSTLENFLERLKTIMREKYSK |
| | CSSGGNGGHKCDITLQEIIKTLNSLTEQKTLCTELTVTDIFAASK |
| | ASGGPEGGSLAALTAHQACHLPLETFTRHRQPRGWEQLEQCGYPV |
| | QRLVALYLAARLSWNQVDQVIRNALASPGSGGDLGEAIREQPEQA |
| | RLALTLAAAESERFVRQGTGNDEAGAANGPADSGDALLERNYPTG |
| | AEFLGDGGDVSFSTRGTQNWTVERLLQAHRQLEERGYVFVGYHGT |
| | FLEAAQSIVFGGVRARSQDLDAIWRGFYIAGDPALAYGYAQDQEP |
| | DARGRIRNGALLRVYVPRSSLPGFYRTSLTLAAPEAAGEVERLIG |
| | HPLPLRLDAITGPEEEGGRLETILGWPLAERTVVIPSAIPTDPRN |
| | VGGDLDPSSIPDKEQAISALPDYASQPGKPPKDEL |

Exemplary IL-13 polypeptides and fusion proteins are provided in Tables 7 and 8 below.

TABLE 7
Exemplary IL-13 Extended Half-Life Fusion Proteins

| SEQ ID NO | |
|---|---|
| (Information) | Amino acid sequence |
| SEQ ID NO: 123 and 124 | Gene 1: |
| Fc-MDNA132-L39/Q111 (1:1 | DKTHTCPPCPAPELLGGPSVFLFPPKPKDTLMISRTPEVTCVVVDVSHEDP |
| KIH) | EVKFNWYVDGVEVHNAKTKPREEQYASTYRVVSVLTVLHQDWLNGKEYKCK |
| (L10H, R86T, D87G, T88R, | VSNKALPAPIEKTISKAKGQPREPQVYTLPPCRDELTKNQVSLWCLVKGFY |
| R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | EELVNITQNQKAPLCNGSMVWSINLTAGMYCAALESLINVSGCSAIEKTQR |
| | MLSGFCPHKVSAGQFSSLHVTGRKIEVAQFVKDLLLHLKKLFKEGQFN |
| | (SEQ ID NO: 123) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPG (SEQ ID NO: 124) |
| SEQ ID NO: 125 | DKTHTCPPCPAPELLGGPSVFLFPPKPKDTLMISRTPEVTCVVVDVSHEDP |
| Fc-MDNA413-R39/Q111 (1:2) | EVKFNWYVDGVEVHNAKTKPREEQYASTYRVVSVLTVLHQDWLNGKEYKCK |
| (L10V, E12A, V18I, R65D, | VSNKALPAPIEKTISKAKGQPREPQVYTLPPSRDELTKNQVSLTCLVKGFY |
| D87S, T88S, L101F, K104R, | PSDIAVEWESNGQPENNYKTTPPVLDSDGSFFLYSKLTVDKSRWQQGNVFS |
| K105T) | CSVMHEALHNHYTQKSLSLSPGGGGGSGGGGSGGGGSPGPVPPSTAVRALI |
| | EELINITQNQKAPLCNGSMVWSINRTAGMYCAALESLINVSGCSAIEKTQD |
| | MLSGFCPHKVSAGQFSSLHVRSSKIEVAQFVKDLLFHLRTLFREGQFN |
| SEQ ID NO: 126 and 124 | Gene 1: |
| MDNA413-R39/Q111-Fc (1:1 | PGPVPPSTAVRALIEELINITQNQKAPLCNGSMVWSINRTAGMYCAALESL |
| KIH) | INVSGCSAIEKTQDMLSGFCPHKVSAGQFSSLHVRSSKIEVAQFVKDLLFH |
| (L10V, E12A, V18I, R65D, | LRTLFREGQFNGGGGSGGGGSGGGGSDKTHTCPPCPAPELLGGPSVFLFPP |
| D87S, T88S, L101F, K104R, | KPKDTLMISRTPEVTCVVVDVSHEDPEVKFNWYVDGVEVHNAKTKPREEQY |
| K105T) | ASTYRVVSVLTVLHQDWLNGKEYKCKVSNKALPAPIEKTISKAKGQPREPQ |
| | VYTLPPCRDELTKNQVSLWCLVKGFYPSDIAVEWESNGQPENNYKTTPPVL |
| | DSDGSFFLYSKLTVDKSRWQQGNVFSCSVMHEALHNHYTQKSLSLSPGK |
| | (SEQ ID NO: 126) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPG (SEQ ID NO: 124) |
| SEQ ID NO: 127 | DKTHTCPPCPAPELLGGPSVFLFPPKPKDTLMISRTPEVTCVVVDVSHEDP |
| Fc-MDNA132-L39/Q111 (1:2) | EVKFNWYVDGVEVHNAKTKPREEQYASTYRVVSVLTVLHQDWLNGKEYKCK |
| (L10H, R86T, D87G, T88R, | VSNKALPAPIEKTISKAKGQPREPQVYTLPPSRDELTKNQVSLTCLVKGFY |
| R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | EELVNITQNQKAPLCNGSMVWSINLTAGMYCAALESLINVSGCSAIEKTQR |
| | MLSGFCPHKVSAGQFSSLHVTGRKIEVAQFVKDLLLHLKKLFKEGQFN |
| SEQ ID NO: 128 | DKTHTCPPCPAPELLGGPSVFLFPPKPKDTLMISRTPEVTCVVVDVSHEDP |
| Fc-MDNA132-R39/Q111 (1:2) | EVKFNWYVDGVEVHNAKTKPREEQYASTYRVVSVLTVLHQDWLNGKEYKCK |
| (L10H, R86T, D87G, T88R, | VSNKALPAPIEKTISKAKGQPREPQVYTLPPSRDELTKNQVSLTCLVKGFY |
| R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | EELVNITQNQKAPLCNGSMVWSINRTAGMYCAALESLINVSGCSAIEKTQR |
| | MLSGFCPHKVSAGQFSSLHVTGRKIEVAQFVKDLLLHLKKLFKEGQFN |
| SEQ ID NO: 129 | DAHKSEVAHRFKDLGEENFKALVLIAFAQYLQQCPFEDHVKLVNEVTEFAK |
| Albumin-MDNA413-R39/Q111 | TCVADESAENCDKSLHTLFGDKLCTVATLRETYGEMADCCAKQEPERNECF |
| (L10V, E12A, V18I, R65D, | LQHKDDNPNLPRLVRPEVDVMCTAFHDNEETFLKKYLYEIARRHPYFYAPE |
| D87S, T88S, L101F, K104R, | LLFFAKRYKAAFTECCQAADKAACLLPKLDELRDEGKASSAKQRLKCASLQ |
| K105T) | KFGERAFKAWAVARLSQRFPKAEFAEVSKLVTDLTKVHTECCHGDLLECAD |
| | DRADLAKYICENQDSISSKLKECCEKPLLEKSHCIAEVENDEMPADLPSLA |
| | ADFVESKDVCKNYAEAKDVFLGMFLYEYARRHPDYSVVLLLRLAKTYETTL |
| | EKCCAAADPHECYAKVFDEFKPLVEEPQNLIKQNCELFEQLGEYKFQNALL |
| | VRYTKKVPQVSTPTLVEVSRNLGKVGSKCCKHPEAKRMPCAEDYLSVVLNQ |
| | LCVLHEKTPVSDRVTKCCTESLVNRRPCFSALEVDETYVPKEFNAETFTFH |
| | ADICTLSEKERQIKKQTALVELVKHKPKATKEQLKAVMDDFAAFVEKCCKA |
| | DDKETCFAEEGKKLVAASQAALGLGGGGSGGGGSGGGGSPGPVPPSTAVRA |
| | LIEELINITQNQKAPLCNGSMVWSINRTAGMYCAALESLINVSGCSAIEKT |
| | QDMLSGFCPHKVSAGQFSSLHVRSSKIEVAQFVKDLLFHLRTLFREGQFN |
| SEQ ID NO: 130 | DKTHTCPPCPAPELLGGPSVFLFPPKPKDTLMISRTPEVTCVVVDVSHEDP |
| Fc-MDNA132-R39/Q111 (1:2) | EVKFNWYVDGVEVHNAKTKPREEQYASTYRVVSVLTVLHQDWLNGKEYKCK |
| (L10H, R86T, D87G, T88R, | VSNKALPAPIEKTISKAKGQPREPQVYTLPPSRDELTKNQVSLTCLVKGFY |
| R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | EELVNITQNQKAPLCNGSMVWSINRTAGMYCAALESLINVSGCSAIEKTQR |
| | MLSGFCPHKVSAGQFSSLHVTGRKIEVAQFVKDLLLHLKKLFKEGQFN |
| SEQ ID NO: 131 | APPCPSCPAPEFLGGPSVFLFPPKPKDTLMISRTPEVTCVVVDVSQEDPEV |
| Fc4-MDNA413-R39/Q111 (1:2) | QFNWYVDGVEVHNAKTKPREEQFNSTYRVVSVLTVLHQDWLNGKEYKCKVS |
| (L10V, E12A, V18I, R65D, | NKGLPSSIEKTISKAKGQPREPQVYTLPPSQEEMTKNQVSLTCLVKGFYPS |
| D87S, T88S, L101F, K104R, | DIAVEWESNGQPENNYKTTPPVLDSDGSFFLYSRLTVDKSRWQEGNVFSCS |
| K105T) | VMHEALHNHYTQKSLSLSPGKGGGSGSPGPVPPSTAVRALIEELINITQNQ |
| | KAPLCNGSMVWSINRTAGMYCAALESLINVSGCSAIEKTQDMLSGFCPHKV |
| | SAGQFSSLHVRSSKIEVAQFVKDLLFHLRTLFREGQFN |
| SEQ ID NO: 132 | DKTHTCPPCPAPELLGGPSVFLFPPKPKDTLMISRTPEVTCVVVDVSHEDP |
| Fc-MDNA413-L39/Q111 (1:2) | EVKFNWYVDGVEVHNAKTKPREEQYASTYRVVSVLTVLHQDWLNGKEYKCK |
| (L10V, E12A, V18I, R65D, | VSNKALPAPIEKTISKAKGQPREPQVYTLPPSRDELTKNQVSLTCLVKGFY |
| D87S, T88S, L101F, K104R, | PSDIAVEWESNGQPENNYKTTPPVLDSDGSFFLYSKLTVDKSRWQQGNVFS |
| K105T) | CSVMHEALHNHYTQKSLSLSPGGGGGSGGGGSGGGGSPGPVPPSTAVRALI |
| | EELINITQNQKAPLCNGSMVWSINLTAGMYCAALESLINVSGCSAIEKTQD |
| | MLSGFCPHKVSAGQFSSLHVRSSKIEVAQFVKDLLFHLRTLFREGQFN |
| SEQ ID NO: 133 AND 134 | Gene 1: |
| MDNA132-L39/R111-Fc (1:1 | PGPVPPSTAHRELIEELVNITQNQKAPLCNGSMVWSINLTAGMYCAALESL |
| KIH) | INVSGCSAIEKTQRMLSGFCPHKVSAGQFSSLHVTGRKIEVAQFVKDLLLH |
| (L10H, R86T, D87G, T88R, | LKKLFKEGRFNGGGGSGGGGSGGGGSDKTHTCPPCPAPELLGGPSVFLFPP |
| R108K) | KPKDTLMISRTPEVTCVVVDVSHEDPEVKFNWYVDGVEVHNAKTKPREEQY |
| | ASTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVL |
| | DSDGSFFLYSKLTVDKSRWQQGNVFSCSVMHEALHNHYTQKSLSLSPG |
| | (SEQ ID NO: 133) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQGNVFS |
| | CSVMHEALHNHYTQKSLSLSPG (SEQ ID NO: 134) |
| SEQ ID NO: 135 | DKTHTCPPCPAPELLGGPSVFLFPPKPKDTLMISRTPEVTCVVVDVSHEDP |
| Fc-MDNA132-L39/R111 (1:2) | EVKFNWYVDGVEVHNAKTKPREEQYASTYRVVSVLTVLHQDWLNGKEYKCK |
| (L10H, R86T, D87G, T88R, | VSNKALPAPIEKTISKAKGQPREPQVYTLPPSRDELTKNQVSLTCLVKGFY |
| R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | EELVNITQNQKAPLCNGSMVWSINLTAGMYCAALESLINVSGCSAIEKTQR |
| | MLSGFCPHKVSAGQFSSLHVTGRKIEVAQFVKDLLLHLKKLFKEGREN |
| SEQ ID NO: 136 | DKTHTCPPCPAPELLGGPSVFLFPPKPKDTLMISRTPEVTCVVVDVSHEDP |
| Fc-MDNA413-L39/R111 (1:2) | EVKFNWYVDGVEVHNAKTKPREEQYASTYRVVSVLTVLHQDWLNGKEYKCK |
| (L10V, E12A, V18I, R65D, | VSNKALPAPIEKTISKAKGQPREPQVYTLPPSRDELTKNQVSLTCLVKGFY |
| D87S, T88S, L101F, K104R, | PSDIAVEWESNGQPENNYKTTPPVLDSDGSFFLYSKLTVDKSRWQQGNVFS |
| K105T) | CSVMHEALHNHYTQKSLSLSPGGGGGSGGGGSGGGGSPGPVPPSTAVRALI |
| | EELINITQNQKAPLCNGSMVWSINLTAGMYCAALESLINVSGCSAIEKTQD |
| | MLSGFCPHKVSAGQFSSLHVRSSKIEVAQFVKDLLFHLRTLFREGREN |
| SEQ ID NO: 138 AND 124 | Gene 1: |
| MDNA413-R39/Q111-Fc (1:1 | PGPVPPSTAVRALIEELINITQNQKAPLCNGSMVWSINRTAGMYCAALESL |
| KIH) | INVSGCSAIEKTQDMLSGFCPHKVSAGQFSSLHVRSSKIEVAQFVKDLLFH |
| (L10V, E12A, V18I, R65D, | LRTLFREGQFNGGGGSGGGGSGGGGSDKTHTCPPCPAPELLGGPSVFLFPP |
| D87S, T88S, L101F, K104R, | KPKDTLMISRTPEVTCVVVDVSHEDPEVKFNWYVDGVEVHNAKTKPREEQY |
| K105T) | ASTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVL |
| | DSDGSFFLYSKLTVDKSRWQQGNVFSCSVMHEALHNHYTQKSLSLSPG |
| | (SEQ ID NO: 137) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPG (SEQ ID NO: 124) |
| SEQ ID NO: 140 | DKTHTCPPCPAPELLGGPSVFLFPPKPKDTLMISRTPEVTCVVVDVSHEDP |
| Fc-MDNA413.18-R39/Q111 | EVKFNWYVDGVEVHNAKTKPREEQYASTYRVVSVLTVLHQDWLNGKEYKCK |
| (1:2) | VSNKALPAPIEKTISKAKGQPREPQVYTLPPSRDELTKNQVSLTCLVKGFY |
| (L10V, E12A, V18H, R65D, | PSDIAVEWESNGQPENNYKTTPPVLDSDGSFFLYSKLTVDKSRWQQGNVFS |
| D87S, T88S, L101F, K104R, | CSVMHEALHNHYTQKSLSLSPGGGGGSGGGGSGGGGSPGPVPPSTAVRALI |
| K105T) | EELHNITQNQKAPLCNGSMVWSINRTAGMYCAALESLINVSGCSAIEKTQD |
| | MLSGFCPHKVSAGQFSSLHVRSSKIEVAQFVKDLLFHLRTLFREGQFN |
| SEQ ID NO: 141 | DKTHTCPPCPAPELLGGPSVFLFPPKPKDTLMISRTPEVTCVVVDVSHEDP |
| Fc-MDNA413.32-R39/Q111 | EVKFNWYVDGVEVHNAKTKPREEQYASTYRVVSVLTVLHQDWLNGKEYKCK |
| (1:2) | VSNKALPAPIEKTISKAKGQPREPQVYTLPPSRDELTKNQVSLTCLVKGFY |
| (L10V, E12A, V18I, S32R, | PSDIAVEWESNGQPENNYKTTPPVLDSDGSFFLYSKLTVDKSRWQQGNVFS |
| R65D, D87S, T88S, L101F, | CSVMHEALHNHYTQKSLSLSPGGGGGSGGGGSGGGGSPGPVPPSTAVRALI |
| K104R, K105T) | EELINITQNQKAPLCNGRMVWSINRTAGMYCAALESLINVSGCSAIEKTQD |
| | MLSGFCPHKVSAGQFSSLHVRSSKIEVAQFVKDLLFHLRTLFREGQFN |
| SEQ ID NO: 142 | DKTHTCPPCPAPELLGGPSVFLFPPKPKDTLMISRTPEVTCVVVDVSHEDP |
| Fc-MDNA413.87-R39/Q111 | EVKFNWYVDGVEVHNAKTKPREEQYASTYRVVSVLTVLHQDWLNGKEYKCK |
| (1:2) | VSNKALPAPIEKTISKAKGQPREPQVYTLPPSRDELTKNQVSLTCLVKGFY |
| (L10V, E12A, V18H, R65D, | PSDIAVEWESNGQPENNYKTTPPVLDSDGSFFLYSKLTVDKSRWQQGNVFS |
| D87H, T88S, L101F, K104R, | CSVMHEALHNHYTQKSLSLSPGGGGGSGGGGSGGGGSPGPVPPSTAVRALI |
| K105T) | EELHNITQNQKAPLCNGSMVWSINRTAGMYCAALESLINVSGCSAIEKTQD |
| | MLSGFCPHKVSAGQFSSLHVRHSKIEVAQFVKDLLFHLRTLFREGQFN |
| SEQ ID NO: 143 | DKTHTCPPCPAPELLGGPSVFLFPPKPKDTLMISRTPEVTCVVVDVSHEDP |
| Fc-MDNA413.94-R39/Q111 | EVKFNWYVDGVEVHNAKTKPREEQYASTYRVVSVLTVLHQDWLNGKEYKCK |
| (1:2) | VSNKALPAPIEKTISKAKGQPREPQVYTLPPSRDELTKNQVSLTCLVKGFY |
| (L10V, E12A, V18I, R65D, | PSDIAVEWESNGQPENNYKTTPPVLDSDGSFFLYSKLTVDKSRWQQGNVFS |
| D87S, T88S, Q94R, L101F, | CSVMHEALHNHYTQKSLSLSPGGGGGSGGGGSGGGGSPGPVPPSTAVRALI |
| K104R, K105T) | EELINITQNQKAPLCNGSMVWSINRTAGMYCAALESLINVSGCSAIEKTQD |
| | MLSGFCPHKVSAGQFSSLHVRSSKIEVARFVKDLLFHLRTLFREGQFN |
| SEQ ID NO: 144 | DKTHTCPPCPAPELLGGPSVFLFPPKPKDTLMISRTPEVTCVVVDVSHEDP |
| Fc-MDNA413.107-R39/Q111 | EVKFNWYVDGVEVHNAKTKPREEQYASTYRVVSVLTVLHQDWLNGKEYKCK |
| (1:2) | VSNKALPAPIEKTISKAKGQPREPQVYTLPPSRDELTKNQVSLTCLVKGFY |
| (L10V, E12A, V18I, R65D, | PSDIAVEWESNGQPENNYKTTPPVLDSDGSFFLYSKLTVDKSRWQQGNVFS |
| D87S, T88S, L101F, K104R, | CSVMHEALHNHYTQKSLSLSPGGGGGSGGGGSGGGGSPGPVPPSTAVRALI |
| K105T, F107W) | EELINITQNQKAPLCNGSMVWSINRTAGMYCAALESLINVSGCSAIEKTQD |
| | MLSGFCPHKVSAGQFSSLHVRSSKIEVAQFVKDLLFHLRTLWREGQFN |
| SEQ ID NO: 145 | DKTHTCPPCPAPELLGGPSVFLFPPKPKDTLMISRTPEVTCVVVDVSHEDP |
| Fc-MDNA413-R39/Q111-RL1 | EVKFNWYVDGVEVHNAKTKPREEQYASTYRVVSVLTVLHQDWLNGKEYKCK |
| (1:2) | VSNKALPAPIEKTISKAKGQPREPQVYTLPPSRDELTKNQVSLTCLVKGFY |
| (L10V, E12A, V18I, R65D, | PSDIAVEWESNGQPENNYKTTPPVLDSDGSFFLYSKLTVDKSRWQQGNVFS |
| D87S, T88S, L101F, K104R, | CSVMHEALHNHYTQKSLSLSPGGGEEEKRKREEEEGSPGPVPPSTAVRALI |
| K105T) | EELINITQNQKAPLCNGSMVWSINRTAGMYCAALESLINVSGCSAIEKTQD |
| | MLSGFCPHKVSAGQFSSLHVRSSKIEVAQFVKDLLFHLRTLFREGQFN |
| SEQ ID NO: 146 | DKTHTCPPCPAPELLGGPSVFLFPPKPKDTLMISRTPEVTCVVVDVSHEDP |
| Fc-MDNA413-R39/Q111-RL2 | EVKFNWYVDGVEVHNAKTKPREEQYASTYRVVSVLTVLHQDWLNGKEYKCK |
| (1:2) | VSNKALPAPIEKTISKAKGQPREPQVYTLPPSRDELTKNQVSLTCLVKGFY |
| (L10V, E12A, V18I, R65D, | PSDIAVEWESNGQPENNYKTTPPVLDSDGSFFLYSKLTVDKSRWQQGNVFS |
| D87S, T88S, L101F, K104R, | CSVMHEALHNHYTQKSLSLSPGEAAAKEAAAKEAAAKPGPVPPSTAVRALI |
| K105T) | EELINITQNQKAPLCNGSMVWSINRTAGMYCAALESLINVSGCSAIEKTQD |
| | MLSGFCPHKVSAGQFSSLHVRSSKIEVAQFVKDLLFHLRTLFREGQFN |
| SEQ ID NO: 147 | PGPVPPSTAVRALIEELINITQNQKAPLCNGSMVWSINRTAGMYCAALESL |
| MDNA413-R39/Q111-Albumin | INVSGCSAIEKTQDMLSGFCPHKVSAGQFSSLHVRSSKIEVAQFVKDLLFH |
| (1:1) | LRTLFREGQFNGGGGSGGGGSGGGGSDAHKSEVAHRFKDLGEENFKALVLI |
| (L10V, E12A, V18I, R65D, | AFAQYLQQCPFEDHVKLVNEVTEFAKTCVADESAENCDKSLHTLFGDKLCT |
| D87S, T88S, L101F, K104R, | VATLRETYGEMADCCAKQEPERNECFLQHKDDNPNLPRLVRPEVDVMCTAF |
| K105T) | HDNEETFLKKYLYEIARRHPYFYAPELLFFAKRYKAAFTECCQAADKAACL |
| | LPKLDELRDEGKASSAKQRLKCASLQKFGERAFKAWAVARLSQRFPKAEFA |
| | EVSKLVTDLTKVHTECCHGDLLECADDRADLAKYICENQDSISSKLKECCE |
| | KPLLEKSHCIAEVENDEMPADLPSLAADFVESKDVCKNYAEAKDVFLGMFL |
| | YEYARRHPDYSVVLLLRLAKTYETTLEKCCAAADPHECYAKVFDEFKPLVE |
| | EPQNLIKQNCELFEQLGEYKFQNALLVRYTKKVPQVSTPTLVEVSRNLGKV |
| | GSKCCKHPEAKRMPCAEDYLSVVLNQLCVLHEKTPVSDRVTKCCTESLVNR |
| | RPCFSALEVDETYVPKEFNAETFTFHADICTLSEKERQIKKQTALVELVKH |
| | KPKATKEQLKAVMDDFAAFVEKCCKADDKETCFAEEGKKLVAASQAALGL |
| SEQ ID NO: 148 and 124 | Gene 1: |
| Fc-MDNA132.89-L39/Q111 (1:1 | DKTHTCPPCPAPELLGGPSVFLFPPKPKDTLMISRTPEVTCVVVDVSHEDP |
| KIH) | EVKFNWYVDGVEVHNAKTKPREEQYASTYRVVSVLTVLHQDWLNGKEYKCK |
| (L10H, R86T, D87G, T88R, | VSNKALPAPIEKTISKAKGQPREPQVYTLPPSRDELTKNQVSLTCLVKGFY |
| K89I, R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | EELVNITQNQKAPLCNGSMVWSINLTAGMYCAALESLINVSGCSAIEKTQR |
| | MLSGFCPHKVSAGQFSSLHVTGRIIEVAQFVKDLLLHLKKLFKEGQFN |
| | (SEQ ID NO: 148) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPG (SEQ ID NO: 124) |
| SEQ ID NO: 1068 and 124 | Gene 1: |
| Fc-MDNA132.90-L39/Q111 (1:1 | DKTHTCPPCPAPELLGGPSVFLFPPKPKDTLMISRTPEVTCVVVDVSHEDP |
| KIH) | EVKFNWYVDGVEVHNAKTKPREEQYASTYRVVSVLTVLHQDWLNGKEYKCK |
| (L10H, R86T, D87G, T88R, | VSNKALPAPIEKTISKAKGQPREPQVYTLPPSRDELTKNQVSLTCLVKGFY |
| I90W, R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | EELVNITQNQKAPLCNGSMVWSINLTAGMYCAALESLINVSGCSAIEKTQR |
| | MLSGFCPHKVSAGQFSSLHVTGRKWEVAQFVKDLLLHLKKLFKEGQFN |
| | (SEQ ID NO: 1068) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPG (SEQ ID NO: 124) |
| SEQ ID NO: 149 and 124 | Gene 1: |
| Fc-MDNA132.15-L39/Q111 (1:1 | DKTHTCPPCPAPELLGGPSVFLFPPKPKDTLMISRTPEVTCVVVDVSHEDP |
| KIH) | EVKFNWYVDGVEVHNAKTKPREEQYASTYRVVSVLTVLHQDWLNGKEYKCK |
| (L10H, E15R, R86T, D87G, | VSNKALPAPIEKTISKAKGQPREPQVYTLPPSRDELTKNQVSLTCLVKGFY |
| T88R, R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | RELVNITQNQKAPLCNGSMVWSINLTAGMYCAALESLINVSGCSAIEKTQR |
| | MLSGFCPHKVSAGQFSSLHVTGRKIEVAQFVKDLLLHLKKLFKEGQFN |
| | (SEQ ID NO: 149) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPG (SEQ ID NO: 124) |
| SEQ ID NO: 150 and 124 | Gene 1: |
| Fc-MDNA132.100-L39/Q111 | DKTHTCPPCPAPELLGGPSVFLFPPKPKDTLMISRTPEVTCVVVDVSHEDP |
| (1:1 KIH) | EVKFNWYVDGVEVHNAKTKPREEQYASTYRVVSVLTVLHQDWLNGKEYKCK |
| (L10H, R86T, D87G, T88R, | VSNKALPAPIEKTISKAKGQPREPQVYTLPPSRDELTKNQVSLTCLVKGFY |
| L100R, R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | EELVNITQNQKAPLCNGSMVWSINLTAGMYCAALESLINVSGCSAIEKTQR |
| | MLSGFCPHKVSAGQFSSLHVTGRKIEVAQFVKDLRLHLKKLFKEGQFN |
| | (SEQ ID NO: 150) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPG (SEQ ID NO: 124) |
| SEQ ID NO: 151 and 124 | Gene 1: |
| Fc-MDNA132.7-L39/Q111 (1:1 | DKTHTCPPCPAPELLGGPSVFLFPPKPKDTLMISRTPEVTCVVVDVSHEDP |
| KIH) | EVKFNWYVDGVEVHNAKTKPREEQYASTYRVVSVLTVLHQDWLNGKEYKCK |
| (S7R, L10H, R86T, D87G, | VSNKALPAPIEKTISKAKGQPREPQVYTLPPSRDELTKNQVSLTCLVKGFY |
| T88R, R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPRTAHRELI |
| | EELVNITQNQKAPLCNGSMVWSINLTAGMYCAALESLINVSGCSAIEKTQR |
| | MLSGFCPHKVSAGQFSSLHVTGRKIEVAQFVKDLLLHLKKLFKEGQFN |
| | (SEQ ID NO: 151) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPG (SEQ ID NO: 124) |
| SEQ ID NO: 152 and 124 | Gene 1: |
| Fc-MDNA132.91-L39/Q111 (1:1 | DKTHTCPPCPAPELLGGPSVFLFPPKPKDTLMISRTPEVTCVVVDVSHEDP |
| KIH) | EVKFNWYVDGVEVHNAKTKPREEQYASTYRVVSVLTVLHQDWLNGKEYKCK |
| (L10H, R86T, D87G, T88R, | VSNKALPAPIEKTISKAKGQPREPQVYTLPPSRDELTKNQVSLTCLVKGFY |
| E91R, R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | EELVNITQNQKAPLCNGSMVWSINLTAGMYCAALESLINVSGCSAIEKTQR |
| | MLSGFCPHKVSAGQFSSLHVTGRKIRVAQFVKDLLLHLKKLFKEGQFN |
| | (SEQ ID NO: 152) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYASTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPG (SEQ ID NO: 124) |
| SEQ ID NO: 153 and 154 | Gene 1: |
| Active Fc-MDNA132.15- | DKTHTCPPCPAPELLGGPSVFLFPPKPKDTLMISRTPEVTCVVVDVSHEDP |
| L39/Q111 (1:1 KIH) | EVKFNWYVDGVEVHNAKTKPREEQYNSTYRVVSVLTVLHQDWLNGKEYKCK |
| (L10H, E15R, R86T, D87G, | VSNKALPAPIEKTISKAKGQPREPQVYTLPPCRDELTKNQVSLWCLVKGFY |
| T88R, R108K) | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTAHRELI |
| | RELVNITQNQKAPLCNGSMVWSINLTAGMYCAALESLINVSGCSAIEKTQR |
| | MLSGFCPHKVSAGQFSSLHVTGRKIEVAQFVKDLLLHLKKLFKEGQFN |
| | (SEQ ID NO: 153) |
| | Gene 2: |
| | DKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCK |
| | VSNKALPAPIEKTISKAKGQPREPQVCTLPPSRDELTKNQVSLSCAVKGFY |
| | PSDIAVEWESNGQPENNYKTTPPVLDSDGSFFLVSKLTVDKSRWQQGNVFS |
| | CSVMHEALHNHYTQKSLSLSPGK (SEQ ID NO: 154) |
| SEQ ID NO: 155 | PGPVPPSTALRELIEELVNITQNQKAPLCNGSMVWSINLTAGMYCAALESL |
| IL13-L39/R111-Fc (WT) (2:1) | INVSGCSAIEKTQRMLSGFCPHKVSAGQFSSLHVRDTKIEVAQFVKDLLLH |
| | LKKLFREGRFNGGGGSGGGGSGGGGSDKTHTCPPCPAPELLGGPSVFLFPP |
| | KPKDTLMISRTPEVTCVVVDVSHEDPEVKFNWYVDGVEVHNAKTKPREEQY |
| | ASTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVL |
| | DSDGSFFLYSKLTVDKSRWQQGNVFSCSVMHEALHNHYTQKSLSLSPGK |
| SEQ ID NO: 156 | DKTHTCPPCPAPELLGGPSVFLFPPKPKDTLMISRTPEVTCVVVDVSHEDP |
| Fc-IL13-L39/Q111 (WT) (1:2) | EVKFNWYVDGVEVHNAKTKPREEQYASTYRVVSVLTVLHQDWLNGKEYKCK |
| | VSNKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQGNVFS |
| | CSVMHEALHNHYTQKSLSLSPGGGGGSGGGGSGGGGSPGPVPPSTALRELI |
| | EELVNITQNQKAPLCNGSMVWSINLTAGMYCAALESLINVSGCSAIEKTQR |
| | MLSGFCPHKVSAGQFSSLHVRDTKIEVAQFVKDLLLHLKKLFREGQFN |
| SEQ ID NO: 157 | PGPVPPSTAHRELIEELVNITQNQKAPLCNGSMVWSINLTAGMYCAALESL |
| MDNA132-L39/Q111 | INVSGCSAIEKTQRMLSGFCPHKVSAGQFSSLHVTGRKIEVAQFVKDLLLH |
| (L10H, R86T, D87G, T88R, | LKKLFKEGQFN |
| R108K) | |
| SEQ ID NO: 158 | PGPVPPSTAHRELIEELVNITQNQKAPLCNGSMVWSINLTAGMYCAALESL |
| MDNA132-L39/R111 | INVSGCSAIEKTQRMLSGFCPHKVSAGQFSSLHVTGRKIEVAQFVKDLLLH |
| (L10H, R86T, D87G, T88R, | LKKLFKEGREN |
| R108K) | |
| SEQ ID NO: 159 | MTAGMYCAALESLINVSGCSAIEKTQRMLSGFCPHKVSAGQFSSLHVTGRK |
| cpMDNA132.15-L39/Q111 | IEVAQFVKDLLLHLKKLFKEGQFNGGNGGPGPVPPSTAHRELIRELVNITQ |
| (L10H, E15R, R86T, D87G, | NQKAPLCNGSMVWSINL |
| T88R, R108K) | |
| SEQ ID NO: 160 | MTAGMYCAALESLINVSGCSAIEKTQRMLSGFCPHKVSAGQFSSLHVTGRK |
| cpMDNA132.15-L39/R111 | IEVAQFVKDLLLHLKKLFKEGRFNGGNGGPGPVPPSTAHRELIRELVNITQ |
| (L10H, E15R, R86T, D87G, | NQKAPLCNGSMVWSINL |
| T88R, R108K) | |
| SEQ ID NO: 161 | MTAGMYCAALESLINVSGCSAIEKTQRMLSGFCPHKVSAGQFSSLHVTGRK |
| cpMDNA132.15-L39/Q111-PE | IEVAQFVKDLLLHLKKLFKEGQFNGGNGGPGPVPPSTAHRELIRELVNITQ |
| (L10H, E15R, R86T, D87G, | NQKAPLCNGSMVWSINLASGGPEGGSLAALTAHQACHLPLETFTRHRQPRG |
| T88R, R108K) | WEQLEQCGYPVQRLVALYLAARLSWNQVDQVIRNALASPGSGGDLGEAIRE |
| | QPEQARLALTLAAAESERFVRQGTGNDEAGAANGPADSGDALLERNYPTGA |
| | EFLGDGGDVSFSTRGTQNWTVERLLQAHRQLEERGYVFVGYHGTFLEAAQS |
| | IVFGGVRARSQDLDAIWRGFYIAGDPALAYGYAQDQEPDARGRIRNGALLR |
| | VYVPRSSLPGFYRTSLTLAAPEAAGEVERLIGHPLPLRLDAITGPEEEGGR |
| | LETILGWPLAERTVVIPSAIPTDPRNVGGDLDPSSIPDKEQAISALPDYAS |
| | QPGKPPKDEL |
| SEQ ID NO: 162 | MTAGMYCAALESLINVSGCSAIEKTQRMLSGFCPHKVSAGQFSSLHVTGRK |
| cpMDNA132.15-L39/R111-PE | IEVAQFVKDLLLHLKKLFKEGRENGGNGGPGPVPPSTAHRELIRELVNITQ |
| (L10H, E15R, R86T, D87G, | NQKAPLCNGSMVWSINLASGGPEGGSLAALTAHQACHLPLETFTRHRQPRG |
| T88R, R108K) | WEQLEQCGYPVQRLVALYLAARLSWNQVDQVIRNALASPGSGGDLGEAIRE |
| | QPEQARLALTLAAAESERFVRQGTGNDEAGAANGPADSGDALLERNYPTGA |
| | EFLGDGGDVSFSTRGTQNWTVERLLQAHRQLEERGYVFVGYHGTFLEAAQS |
| | IVFGGVRARSQDLDAIWRGFYIAGDPALAYGYAQDQEPDARGRIRNGALLR |
| | VYVPRSSLPGFYRTSLTLAAPEAAGEVERLIGHPLPLRLDAITGPEEEGGR |
| | LETILGWPLAERTVVIPSAIPTDPRNVGGDLDPSSIPDKEQAISALPDYAS |
| | QPGKPPKDEL |
| SEQ ID NO: 163 | GPGPVPPSTAHRELIRELVNITQNQKAPLCNGSMVWSINLTAGMYCAALES |
| MDNA132.15-L39/Q111-PE | LINVSGCSAIEKTQRMLSGFCPHKVSAGQFSSLHVTGRKIEVAQFVKDLLL |
| (L10H, E15R, R86T, D87G, | HLKKLFKEGQFNASGGPEGGSLAALTAHQACHLPLETFTRHRQPRGWEQLE |
| T88R, R108K) | QCGYPVQRLVALYLAARLSWNQVDQVIRNALASPGSGGDLGEAIREQPEQA |
| | RLALTLAAAESERFVRQGTGNDEAGAANGPADSGDALLERNYPTGAEFLGD |
| | GGDVSFSTRGTQNWTVERLLQAHRQLEERGYVFVGYHGTFLEAAQSIVFGG |
| | VRARSQDLDAIWRGFYIAGDPALAYGYAQDQEPDARGRIRNGALLRVYVPR |
| | SSLPGFYRTSLTLAAPEAAGEVERLIGHPLPLRLDAITGPEEEGGRLETIL |
| | GWPLAERTVVIPSAIPTDPRNVGGDLDPSSIPDKEQAISALPDYASQPGKP |
| | PKDEL |
| SEQ ID NO: 164 | GPGPVPPSTAHRELIRELVNITQNQKAPLCNGSMVWSINLTAGMYCAALES |
| MDNA132.15-R111-PE | LINVSGCSAIEKTQRMLSGFCPHKVSAGQFSSLHVTGRKIEVAQFVKDLLL |
| (L10H, E15R, R86T, D87G, | HLKKLFKEGRFNASGGPEGGSLAALTAHQACHLPLETFTRHRQPRGWEQLE |
| T88R, R108K) | QCGYPVQRLVALYLAARLSWNQVDQVIRNALASPGSGGDLGEAIREQPEQA |
| | RLALTLAAAESERFVRQGTGNDEAGAANGPADSGDALLERNYPTGAEFLGD |
| | GGDVSFSTRGTQNWTVERLLQAHRQLEERGYVFVGYHGTFLEAAQSIVFGG |
| | VRARSQDLDAIWRGFYIAGDPALAYGYAQDQEPDARGRIRNGALLRVYVPR |
| | SSLPGFYRTSLTLAAPEAAGEVERLIGHPLPLRLDAITGPEEEGGRLETIL |
| | GWPLAERTVVIPSAIPTDPRNVGGDLDPSSIPDKEQAISALPDYASQPGKP |
| | PKDEL |

TABLE 8
Exemplary IL-13 anti-PD-1 Fusion Proteins

| SEQ ID NO | |
|---|---|
| (Information) | Amino acid sequence |
| SEQ ID NOs: 194, 174, and | Gene 1: |
| 175 | EVQLQESGPGLVKPSQSLSLTCSVTGYSITSSYRWNWIRKFPGNRLEWMGY |
| Anti-mPD1-MDNA132-L39/Q111 | INSAGISNYNPSLKRRISITRDTSKNQFFLQVNSVTTEDAATYYCARSDNM |
| (KIH) | GTTPFTYWGQGTLVTVSSASTKGPSVFPLAPSSKSTSGGTAALGCLVKDYF |
| (L10H, R86T, D87G, T88R, | PEPVTVSWNSGALTSGVHTFPAVLQSSGLYSLSSVVTVPSSSLGTQTYICN |
| R108K) | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGGGGGSGGGGS |
| | GGGGSPGPVPPSTAHRELIEELVNITQNQKAPLCNGSMVWSINLTAGMYCA |
| | ALESLINVSGCSAIEKTQRMLSGFCPHKVSAGQFSSLHVTGRKIEVAQFVK |
| | DLLLHLKKLFKEGQFN (SEQ ID NO: 194) |
| | Gene 2: |
| | EVQLQESGPGLVKPSQSLSLTCSVTGYSITSSYRWNWIRKFPGNRLEWMGY |
| | INSAGISNYNPSLKRRISITRDTSKNQFFLQVNSVTTEDAATYYCARSDNM |
| | GTTPFTYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVCTLPPS |
| | RDELTKNQVSLSCAVKGFYPSDIAVEWESNGQPENNYKTTPPVLDSDGSFF |
| | LVSKLTVDKSRWQQGNVFSCSVMHEALHNHYTQKSLSLSPGK (SEQ ID |
| | NO: 174) |
| | Gene 3: |
| | DIVMTQGTLPNPVPSGESVSITCRSSKSLLYSDGKTYLNWYLQRPGQSPQL |
| | LIYWMSTRASGVSDRFSGSGSGTDFTLKISGVEAEDVGIYYCQQGLEFPTF |
| | GGGTKLELKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQG |
| | LSSPVTKSFNRGEC (SEQ ID NO: 175) |
| SEQ ID NOs: 195, 177, and | Gene 1: |
| 178 | QVQLVESGGGVVQPGRSLRLDCKASGITFSNSGMHWVRQAPGKGLEWVAVI |
| Anti-huPD1-MDNA132-L39/Q111 | WYDGSKRYYADSVKGRFTISRDNSKNTLFLQMNSLRAEDTAVYYCATNDDY |
| (KIH) | WGQGTLVTVSSASTKGPSVFPLAPCSRSTSESTAALGCLVKDYFPEPVTVS |
| (L10H, R86T, D87G, T88R, | WNSGALTSGVHTFPAVLQSSGLYSLSSVVTVPSSSLGTKTYTCNVDHKPSN |
| R108K) | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVYTLPPCRDELTKN |
| | QVSLWCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSPG |
| | PVPPSTAHRELIEELVNITQNQKAPLCNGSMVWSINLTAGMYCAALESLIN |
| | VSGCSAIEKTQRMLSGFCPHKVSAGQFSSLHVTGRKIEVAQFVKDLLLHLK |
| | KLFKEGQFN (SEQ ID NO: 195) |
| | Gene 2: |
| | QVQLVESGGGVVQPGRSLRLDCKASGITFSNSGMHWVRQAPGKGLEWVAVI |
| | WYDGSKRYYADSVKGRFTISRDNSKNTLFLQMNSLRAEDTAVYYCATNDDY |
| | WGQGTLVTVSSASTKGPSVFPLAPCSRSTSESTAALGCLVKDYFPEPVTVS |
| | WNSGALTSGVHTFPAVLQSSGLYSLSSVVTVPSSSLGTKTYTCNVDHKPSN |
| | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVCTLPPSRDELTKN |
| | QVSLSCAVKGFYPSDIAVEWESNGQPENNYKTTPPVLDSDGSFFLVSKLTV |
| | DKSRWQQGNVFSCSVMHEALHNHYTQKSLSLSPGK (SEQ ID NO: |
| | 177) |
| | Gene 3: |
| | EIVLTQSPATLSLSPGERATLSCRASQSVSSYLAWYQQKPGQAPRLLIYDA |
| | SNRATGIPARFSGSGSGTDFTLTISSLEPEDFAVYYCQQSSNWPRTFGQGT |
| | KVEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGEC (SEQ ID NO: 178) |
| SEQ ID NOs: 196 and 175 | Gene 1&2: |
| Anti-mPD1-MDNA413-R39 /Q111 | EVQLQESGPGLVKPSQSLSLTCSVTGYSITSSYRWNWIRKFPGNRLEWMGY |
| (1:2) | INSAGISNYNPSLKRRISITRDTSKNQFFLQVNSVTTEDAATYYCARSDNM |
| (L10V, E12A, V18I, R65D, | GTTPFTYWGQGTLVTVSSASTKGPSVFPLAPSSKSTSGGTAALGCLVKDYF |
| D87S, T88S, L101F, K104R, | PEPVTVSWNSGALTSGVHTFPAVLQSSGLYSLSSVVTVPSSSLGTQTYICN |
| K105T) | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQGNVFSCSVMHEALHNHYTQKSLSLSPGGGGGSGGGGS |
| | GGGGSPGPVPPSTAVRALIEELINITQNQKAPLCNGSMVWSINRTAGMYCA |
| | ALESLINVSGCSAIEKTQDMLSGFCPHKVSAGQFSSLHVRSSKIEVAQFVK |
| | DLLFHLRTLFREGQFN (SEQ ID NO: 196) |
| | Gene 3: |
| | DIVMTQGTLPNPVPSGESVSITCRSSKSLLYSDGKTYLNWYLQRPGQSPQL |
| | LIYWMSTRASGVSDRFSGSGSGTDFTLKISGVEAEDVGIYYCQQGLEFPTF |
| | GGGTKLELKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQG |
| | LSSPVTKSFNRGEC (SEQ ID NO: 175) |
| SEQ ID NOs: 197 and 178 | Gene 1&2: |
| Anti-huPD1-MDNA413-R39/Q111 | QVQLVESGGGVVQPGRSLRLDCKASGITFSNSGMHWVRQAPGKGLEWVAVI |
| (1:2) | WYDGSKRYYADSVKGRFTISRDNSKNTLFLQMNSLRAEDTAVYYCATNDDY |
| (L10V, E12A, V18I, R65D, | WGQGTLVTVSSASTKGPSVFPLAPCSRSTSESTAALGCLVKDYFPEPVTVS |
| D87S, T88S, L101F, K104R, | WNSGALTSGVHTFPAVLQSSGLYSLSSVVTVPSSSLGTKTYTCNVDHKPSN |
| K105T) | TKVDKRVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVYTLPPSREEMTKN |
| | QVSLTCLVKGFYPSDIAVEWESNGQPENNYKTTPPVLDSDGSFFLYSKLTV |
| | DKSRWQQGNVFSCSVMHEALHNHYTQKSLSLSPGGGGGSGGGGSGGGGSPG |
| | PVPPSTAVRALIEELINITQNQKAPLCNGSMVWSINRTAGMYCAALESLIN |
| | VSGCSAIEKTQDMLSGFCPHKVSAGQFSSLHVRSSKIEVAQFVKDLLFHLR |
| | TLFREGQFN (SEQ ID NO: 197) |
| | Gene 3: |
| | EIVLTQSPATLSLSPGERATLSCRASQSVSSYLAWYQQKPGQAPRLLIYDA |
| | SNRATGIPARFSGSGSGTDFTLTISSLEPEDFAVYYCQQSSNWPRTFGQGT |
| | KVEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGEC (SEQ ID NO: 178) |
| SEQ ID NOs: 198, 174, and | Gene 1: |
| 175 | EVQLQESGPGLVKPSQSLSLTCSVTGYSITSSYRWNWIRKFPGNRLEWMGY |
| Anti-mPD1-MDNA132.15 (1:1 | INSAGISNYNPSLKRRISITRDTSKNQFFLQVNSVTTEDAATYYCARSDNM |
| KIH) | GTTPFTYWGQGTLVTVSSASTKGPSVFPLAPSSKSTSGGTAALGCLVKDYF |
| (L10H, E15R, R86T, D87G, | PEPVTVSWNSGALTSGVHTFPAVLQSSGLYSLSSVVTVPSSSLGTQTYICN |
| T88R, R108K) | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGGGGGSGGGGS |
| | GGGGSPGPVPPSTAHRELIRELVNITQNQKAPLCNGSMVWSINLTAGMYCA |
| | ALESLINVSGCSAIEKTQRMLSGFCPHKVSAGQFSSLHVTGRKIEVAQFVK |
| | DLLLHLKKLFKEGQFN (SEQ ID NO: 198) |
| | Gene 2: |
| | EVQLQESGPGLVKPSQSLSLTCSVTGYSITSSYRWNWIRKFPGNRLEWMGY |
| | INSAGISNYNPSLKRRISITRDTSKNQFFLQVNSVTTEDAATYYCARSDNM |
| | GTTPFTYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYASTYRVV |
| | SVLTVLHQDWLNGKEYKCKVSNKALPAPIEKTISKAKGQPREPQVCTLPPS |
| | RDELTKNQVSLSCAVKGFYPSDIAVEWESNGQPENNYKTTPPVLDSDGSFF |
| | LVSKLTVDKSRWQQGNVFSCSVMHEALHNHYTQKSLSLSPGK (SEQ ID |
| | NO: 174) |
| | Gene 3: |
| | DIVMTQGTLPNPVPSGESVSITCRSSKSLLYSDGKTYLNWYLQRPGQSPQL |
| | LIYWMSTRASGVSDRFSGSGSGTDFTLKISGVEAEDVGIYYCQQGLEFPTF |
| | GGGTKLELKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQG |
| | LSSPVTKSFNRGEC (SEQ ID NO: 175) |

### 1. Cargo Moieties

In some embodiments, the IL-4s and IL-13s disclosed herein are linked to a cargo moiety that functions to reduce or inhibit cancer stem cell growth, or kill cancer cells and/or cancer stem cells. Without being bound by any particular theory of operation, it is believed that IL-4s or IL-13s linked to such a cargo moiety are capable of targeting the cargo moiety to cancer cells that overexpress IL-4R or IL-13 receptors, thereby allowing the linked cargo moiety to act locally in the tumor microenvironment to promote anti-tumor activity. Thus, the IL-4 and IL-13 functions as a targeting moiety for the cargo moiety.

In some examples cargo moieties are not proteins, but other molecules that reduce or inhibit cancer stem cell growth, or kill cancer cells and/or cancer stem cells, such as chemotherapeutic agents. In some examples, cargo moieties also reduce or inhibit bulk cancer cell growth, or kill cancer cells. Any protein or other agent that functions to reduce or inhibit cancer stem cell growth, or kill such cells, can be used as a cargo moiety. For example, toxins and proteins that function to control cell life cycles can be used as cargo moieties. Toxins that can be used as cargo moieties include toxins made by microorganisms, plants or animals, as well as toxins made by human cells. Similarly, any natural cell growth controlling protein can be used as a cargo moiety. For example, proteins that trigger cell death during the normal life cycle of an organism can be used as cargo moieties. In some examples, an oncolytic virus (e.g., see Allen et al., Mol. Ther. 16:1556-64, 2008) or liposomes carrying cytotoxic agents (e.g., see Madhankumar et al., Mol. Cancer. Ther. 5:3162-9, 2006) is used as the cargo protein. As disclosed herein the tumor growth inhibitory activity of such IL-4s and IL-13s linked to cargo moieties are advantageously enhanced when administered in combination with an immunomodulatory agent such as any of the IL-2 muteins provided herein (see Example 6). Exemplary cargo moieties are provided in Table 9 below:

TABLE 9
Exemplary cargo moiety sequences

| Cargo Moiety | Accession Numbers* |
|---|---|
| Aerolysin | ABR14715.1; ABR14714.1 |
| Proaerolysin | AAA21938.1; P09167.2; U.S. Pat. No. 7,282,476 |
| | (proaerolysin sequences therein herein |
| | incorporated by reference) |
| Bouganin | AAL35962 and SEQ ID NO: 9 in U.S. Pat. No. |
| | 6,737,511, as well as variant sequences |
| | provided in U.S. Pat. No. 7,339,031 and WO |
| | 2005/090579 (bouganin sequences therein |
| | herein incorporated by reference) |
| Pseudomonas | 1IKP A; AAB59097.1; AAF90003.1 (also see |
| exotoxin | SEQ ID NO: 1 of U.S. Pat. No. 6,011,002) |
| Cholera toxin | BAA06291.1; ACF35010.1; BAA06288.1; as well |
| | as variant sequences provided in U.S. patent |
| | application Ser. No. 61/058,872 (variant |
| | cholera toxin sequences therein herein |
| | incorporated by reference) |
| Ribonuclease A | BAA05124.1; NP_937877.1; NP_115961.2; |
| | Q5GAN4.1; and sequences provided in PCT |
| | Publication No. WO2007/041361 (rapLR1 |
| | sequences therein herein incorporated by |
| | reference) |

IL-4s and IL-13s can be linked to a cargo moiety using any suitable method known in the art, depending on the cargo moiety used. In some embodiments, the cargo moiety is directly attached to the IL-4 or IL-13. In some embodiments, the cargo moiety is attached to the IL-4 or IL-13 using a suitable linker. Suitable linkers for attaching the cargo moiety to the IL-4s and IL-13s are further described below.

In some embodiments, the cargo moiety is a Pseudomonas exotoxin (PE). PE is a toxin secreted by Pseudomonas. Native PE is cytotoxic for mammalian cells due to its ability to enter cells by receptor-mediated endocytosis and then, after a series of intracellular processing steps, translocate to the cell cytosol and ADP-ribosylate elongation factor 2. This results in the inhibition of protein synthesis and cell death. PE has three functional domains: an amino-terminal receptor-binding domain, a middle translocation domain, and a carboxyl-terminal ADP-ribosylation domain. Modified PE molecules can include elimination of domain Ia, as well as deletions in domains II and III. Exemplary PE proteins that can be used in the targeted cargo proteins of the present disclosure include those provided in Table 1, as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native PE protein.

In one example, the cargo moiety is a toxin. Exemplary toxins that can be used include pore-forming toxins, and toxins that upon internalization inhibit cell growth. In other examples, cargo moieties are proteins that are apoptotic triggering proteins, and cell growth inhibiting proteins. In some examples, the toxin is a modified bacterial toxin such that the resulting toxin is less immunogenic than the native toxin. Such modified toxins, such as a modified Pseudomonas exotoxin A, can reduce the patient's immunogenic response, thereby allowing repeated administration.

Pore forming toxins are toxins that form pores in the cell membrane thereby killing the cell via cell lyses. Exemplary pore forming toxins include but are not limited to human toxins such as perforin or bacterial toxins such as aerolysin as well as modified pore-forming protein toxins that are derived from naturally occurring pore-forming protein toxins (nPPTs) such as aerolysin or aerolysin-related polypeptides. Suitable aerolysin-related nPPTs have the following features: a pore-forming activity that is activated by removal of an inhibitory domain via protease cleavage, and the ability to bind to receptors that are present on cell membranes through one or more binding domains. In some examples the linker can be engineered to be sensitive to a protease or be chemically liable. Additional examples of pore forming toxins that can be used as cargo moieties include, but are not limited to, proaerolysin from Aeromonas hydrophila, Aeromonas trota and Aeromonas salmonicida, alpha toxin from Clostridium septicum, anthrax protective antigen, Vibrio cholerae VCC toxin, epsilon toxin from Clostridium perfringens, and Bacillus thuringiensis delta toxins. A detailed description of the engineering of proaerolysin can be found in U.S. Pat. No. 7,282,476, which is herein incorporated by reference.

Additional toxins that can be used as cargo moieties include toxins that act within a cell. For example, anthrax, diphtheria, cholera, and botulinum toxins include a portion that acts in the cytoplasm, as well as a portion that acts to bind to the cell surface. These toxins, or portions thereof, can be linked to a targeting moiety and used to inhibit cancer stem cell growth. Select members of the ribonuclease A (RNase A) superfamily are potent cytotoxins. These cytotoxic ribonucleases enter the cytosol, where they degrade cellular RNA and cause cell death.

In some examples ribosome inactivating proteins can be used as toxins. In these examples the cargo moiety is a polypeptide having ribosome-inactivating activity including, without limitation, gelonin, bouganin, saporin, ricin, ricin A chain, bryodin, restrictocin, and variants thereof. Diphtheria toxin and Pseudomonas exotoxin A inhibit protein synthesis via ADP-ribosylation of elongation factor 2. When the cargo moiety is a ribosome-inactivating protein or inhibits protein synthesis via ADP-ribosylation of elongation factor 2, the cargo protein targeting IL-4R can be internalized upon binding to the cancer stem cell. Cargo moieties that induce apoptosis can also be used to target cancer cells and/or cancer stem cells. Examples of cargo moieties that induce apoptosis include caspases, granzymes and BCL-2 pro-apoptotic related proteins such as BAX (e.g., Accession no: CAE52910), BAD (e.g., Accession no: CAG46757), BAT (e.g., Accession no: AA107425), BAK (e.g., Accession no: AAA74466), BIK (e.g., Accession no: CAG30276), BOK (e.g., Accession no: AAH06203), BID (e.g., Accession no: CAG28531), BIM (e.g., Accession no: NP_619527) and BMF (e.g., Accession no: AAH69328). These cargo moieties can be used alone of in combination to reduce or inhibit cancer stem cell growth.

Aerolysin is a channel-forming toxin produced as an inactive protoxin called proaerolysin (PA). Exemplary aerolysin and PA sequences that can be used in a cargo protein targeting IL-4R are provided in Table 1. The PA protein contains many discrete functionalities that include a binding domain, a toxin domain, and a C-terminal inhibitory peptide domain that contains a protease activation site. The binding domain recognizes and binds to glycophosphatidylinositol (GPI) membrane anchors, such as are found in Thy-1 on T lymphocytes, the PIGA gene product found in erythrocyte membranes and Prostate Stem Cell Antigen (PSCA). The activation or proteolysis site within proaerolysin is a six amino acid sequence that is recognized as a proteolytic substrate by the furin family of proteases. PA is activated upon hydrolysis of a C-terminal inhibitory segment by furin. Activated aerolysin binds to GPI-anchored proteins in the cell membrane and forms a heptamer that inserts into the membrane producing well-defined channels of about 17 angstroms. Channel formation leads to rapid cell death. Wild-type aerolysin is toxic to mammalian cells, including erythrocytes, for example at 1 nanomolar or less.

In some examples, a target cargo protein is an PA molecule with the native furin site replaced with a different cleavage site, such as prostate-specific protease cleavage site (e.g., a PSA-specific cleavage site, which permits activation of the variant PA in the presence of a prostate-specific protease such as PSA, PMSA, or HK2). In one example, a prostate-specific protease cleavage site is inserted into the native furin cleavage site of PA, such that PA is activated in the presence of a prostate-specific protease, but not furin. In another example, a variant PA molecule further includes a functionally deleted binding domain (e.g., about amino acids 1-83 of a native PA protein sequence). Functional deletions can be made using any method known in the art, such as deletions, insertions, mutations, or substitutions. In some examples, IL-4 receptor targeted cargo proteins include variant PA molecules in which the native binding domain is functionally deleted and replaced with a prostate-tissue or other tissue-specific binding domain. In other examples, variant PA molecules include a furin cleavage site and a functionally deleted binding domain which is replaced with a prostate-tissue specific binding domain. Such variant PA molecules are targeted to prostate cells via the prostate-tissue specific binding domain, and activated in the presence of furin.

Bouganin is a ribosome-binding protein originally isolated from Bougainvillea speotabilis (see U.S. Pat. No. 6,680,296). Exemplary modified bouganins are described in WO 2005/090579 and U.S. Pat. No. 7,339,031. Bouganin damages ribosomes and leads to a cessation of protein synthesis and cell death. Exemplary bouganin proteins that can be used in the IL-4 receptor targeted cargo proteins of the present disclosure include those in GenBank Accession No. AAL35962, as well as those native and modified bouganin sequences provided in U.S. Pat. Nos. 6,680,296; 7,339,031 and PCT publication WO 2005/090579 (bouganin sequences herein incorporated by reference), as well as sequences having at least 60% sequence identity, at least 75%, at least 80%, at least 85%, at least 90%, at least 95%, at least 98% or even at least 99% sequence identity to such sequences.

Thapsigargin is an inhibitor of sarco/endoplasmic reticulum Ca2+ ATPases. Thapsigargin is classified as a sesquiterpene lactone, and raises cytosolic calcium concentration by blocking the ability of the cell to pump calcium into the sarcoplasmic and endoplasmic reticulum which causes these stores to become depleted. Store-depletion can secondarily activate plasma membrane calcium channels, allowing an influx of calcium into the cytosol.

Ribonuclease A (RNAse A) is an endonuclease that cleaves single-stranded RNA. RNAse A toxins can be obtained from mammals and reptiles. Exemplary RNAse A proteins that can be used in the IL-4R or IL-13 receptor targeted cargo proteins of the present disclosure include those provided in Table 1, as well as sequences having at least 60% sequence identity, at least 75%, at least 80%, at least 85%, at least 90%, at least 95%, at least 98% or even at least 99% sequence identity to such sequences, as long as the variant retains or has enhanced biological activity of the native RNAseA toxin.

The cargo moiety used can include native sequences (such as the GenBank Accession Nos. and sequences present in the patents referenced in Table 1 and listed above), as well as variants thereof, such as a variant having at least 98%, at least 95%, at least 90%, at least 80%, at least 70%, or at least 60% sequence identity with the native cargo moiety, as long as the variant retains or has enhanced biological activity of the native cargo moiety (e.g., at least about this amount of sequence identity to the GenBank Accession Nos. listed in Table 1 and listed above). In some examples, variant sequences retain substantially the same amount (or even more) of the native biological function of the cargo moiety, such as the ability to kill or inhibit the growth of a cancer stem cell. A cargo moiety can also be a fragment of the native sequence that retains a substantial amount of the native biological function of the protein.

### 2. Cargo Moiety Linkers

Linking of a cargo moiety to a targeting moiety (e.g., IL-4 or IL-13) may be direct meaning that one portion of the cargo moiety is directly attached to a portion of the targeting moiety. For example, one end of the amino acid sequence of a cargo protein can be directly attached to an end of the amino acid sequence of the targeting moiety. For example, the C-terminus of the cargo protein can be linked to the N-terminus of the targeting moiety, or the C-terminus of the targeting moiety can be linked to the N-terminus of the cargo protein. Methods of generating such fusion proteins are routine in the art, for example using recombinant molecular biology methods.

In another example, the cargo moiety is linked to the targeting moiety indirectly through a linker. The linker can serve, for example, simply as a convenient way to link the two entities, as a means to spatially separate the two entities, to provide an additional functionality to the IL-4 receptor targeted cargo protein, or a combination thereof.

In general, the linker joining the targeting moiety (e.g., IL-4 or IL-13) and the cargo moiety can be designed to (1) allow the two molecules to fold and act independently of each other, (2) not have a propensity for developing an ordered secondary structure which could interfere with the functional domains of the two moieties, (3) have minimal hydrophobic or charged characteristic which could interact with the functional protein domains and/or (4) provide steric separation of the two regions. For example, in some instances it may be desirable to spatially separate the targeting moiety and the cargo moiety to prevent the targeting moiety from interfering with the inhibitory activity of the targeted cargo moiety and/or the cargo moiety interfering with the targeting activity of the targeting moiety. The linker can also be used to provide, for example, lability to the connection between the targeting moiety and the cargo moiety, an enzyme cleavage site (for example a cleavage site for a protease), a stability sequence, a molecular tag, a detectable label, or various combinations thereof.

The linker can be bifunctional or polyfunctional, e.g. contains at least about a first reactive functionality at, or proximal to, a first end of the linker that is capable of bonding to, or being modified to bond to, the targeting moiety and a second reactive functionality at, or proximal to, the opposite end of the linker that is capable of bonding to, or being modified to bond to, the cargo moiety being modified. The two or more reactive functionalities can be the same (i.e. the linker is homobifunctional) or they can be different (i.e. the linker is heterobifunctional). A variety of bifunctional or polyfunctional cross-linking agents are known in the art that are suitable for use as linkers (for example, those commercially available from Pierce Chemical Co., Rockford, III.), such as avidin and biotin. Alternatively, these reagents can be used to add the linker to the targeting moiety and/or cargo moiety.

The length and composition of the linker can be varied considerably provided that it can fulfill its purpose as a molecular bridge. The length and composition of the linker are generally selected taking into consideration the intended function of the linker, and optionally other factors such as ease of synthesis, stability, resistance to certain chemical and/or temperature parameters, and biocompatibility. For example, the linker should not significantly interfere with the ability of the targeting moiety to target the linked cargo protein to a cancer stem cell, or with the activity of the linked cargo protein relating to activation, pore-forming ability, or toxin activity.

Linkers suitable for use may be branched, unbranched, saturated, or unsaturated hydrocarbon chains, as well as peptides as noted above. Furthermore, if the linker is a peptide, the linker can be attached to the targeting moiety and/or the cargo moiety using recombinant DNA technology. Such methods are well-known in the art and details of this technology can be found, for example, in Sambrook et al., supra.

In one example, the linker is a branched or unbranched, saturated or unsaturated, hydrocarbon chain having from 1 to 100 carbon atoms, wherein one or more of the carbon atoms is optionally replaced by —O— or —NR— (wherein R is H, or C1 to C6 alkyl), and wherein the chain is optionally substituted on carbon with one or more substituents selected from the group of (C1-C6) alkoxy, (C3-C6) cycloalkyl, (C1-C6) alkanoyl, (C1-C6) alkanoyloxy, (C1-C6) alkoxycarbonyl, (C1-C6) alkylthio, amide, azido, cyano, nitro, halo, hydroxy, oxo (.dbd.O), carboxy, aryl, aryloxy, heteroaryl, and heteroaryloxy.

Examples of suitable linkers include, but are not limited to, peptides having a chain length of 1 to 500 amino acid residues (such as 1 to 100, 1 to 50, 6 to 30, such as less than 30 amino acids). Typically surface amino acids in flexible protein regions include Gly, Asn and Ser. Other neutral amino acids, such as Thr and Ala, can also be used in the linker sequence. Additional amino acids can be included in the linker to provide unique restriction sites in the linker sequence to facilitate construction of the fusions. Other exemplary linkers include those derived from groups such as ethanolamine, ethylene glycol, polyethylene with a chain length of 6 to 100 carbon atoms, polyethylene glycol with 3 to 30 repeating units, phenoxyethanol, propanolamide, butylene glycol, butyleneglycolamide, propyl phenyl, and ethyl, propyl, hexyl, steryl, cetyl, and palmitoyl alkyl chains.

In one example, the linker is a branched or unbranched, saturated or unsaturated, hydrocarbon chain, having from 1 to 50 carbon atoms, wherein one or more of the carbon atoms is optionally replaced by —O— or —NR— (wherein R is as defined above), and wherein the chain is optionally substituted on carbon with one or more substituents selected from the group of (C1-C6) alkoxy, (C1-C6) alkanoyl, (C1-C6) alkanoyloxy, (C1-C6) alkoxycarbonyl, (C1-C6) alkylthio, amide, hydroxy, oxo (.dbd.O), carboxy, aryl and aryloxy.

In a specific example, the linker is a peptide having a chain length of 1 to 50 amino acid residues, such as 1 to 40, 1 to 20, or 5 to 10 amino acid residues.

Peptide linkers that are susceptible to cleavage by enzymes of the complement system, urokinase, tissue plasminogen activator, trypsin, plasmin, or another enzyme having proteolytic activity may be used in one example. According to another example, the IL-4 receptor targeted cargo protein includes a targeting moiety attached via a linker susceptible to cleavage by enzymes having a proteolytic activity such as a urokinase, a tissue plasminogen activator, plasmin, thrombin or trypsin. In addition, targeting moieties may be attached to the cargo moiety via disulfide bonds (for example, the disulfide bonds on a cysteine molecule). Since many tumors naturally release high levels of glutathione (a reducing agent) this can reduce the disulfide bonds with subsequent release of the cargo moiety at the site of delivery.

In one example, the IL-4 receptor targeted or IL-13 receptor targeted cargo protein includes a targeting moiety linked by a cleavable linker region. In another example, the cleavable linker region is a protease-cleavable linker, although other linkers, cleavable for example by small molecules, may be used. Examples of protease cleavage sites are those cleaved by factor Xa, thrombin and collagenase. In one example, the protease cleavage site is one that is cleaved by a protease that is associated with a disease. In another example, the protease cleavage site is one that is cleaved by a protease that is up-regulated or associated with cancers in general. Examples of such proteases are uPA, the matrix metalloproteinase (MMP) family, the caspases, elastase, prostate specific antigen (PSA, a serine protease), and the plasminogen activator family, as well as fibroblast activation protein. In still another example, the cleavage site is cleaved by a protease secreted by cancer-associated cells. Examples of these proteases include matrixmetalloproteases, elastase, plasmin, thrombin, and uPA. In another example, the protease cleavage site is one that is up-regulated or associated with a specific cancer. The precise sequences are available in the art and the skilled person will have no difficulty in selecting a suitable cleavage site. By way of example, the protease cleavage region targeted by Factor Xa is I E G R. The protease cleavage region targeted by enterokinase is D D D D K. The protease cleavage region targeted by thrombin is L V P R G. In one example, the cleavable linker region is one which is targeted by endocellular proteases.

As known in the art, the attachment of a linker to cargo moiety (or of a linker element to a cleavable element, or a cleavable element to another cargo moiety) need not be a particular mode of attachment or reaction.

### D. Recombinant Expression of IL-2, IL-4, OR IL-13 Mutein Molecule, Expression Vectors and Host Cells

In various embodiments, polypeptides used in the practice of the instant invention are synthetic or are produced by expression of a recombinant nucleic acid molecule. In the event the polypeptide is a chimera (e.g., a fusion protein containing at least a mutant IL-2 polypeptide and a heterologous polypeptide, including a IL-2 cytokine fusion), it can be encoded by a hybrid nucleic acid molecule containing one sequence that encodes all or part of the IL-2, IL-4, or IL-13 mutein molecule, and a second sequence that encodes all or part of the heterologous polypeptide. For example, subject IL-2, IL-4, or IL-13 mutein molecules described herein may be fused to a hexa-histidine (SEQ ID NO: 1063) tag to facilitate purification of bacterially expressed protein, or to a hemagglutinin tag to facilitate purification of protein expressed in eukaryotic cells.

Methods for constructing a DNA sequence encoding the IL-2, IL-4, or IL-13 mutein molecules and expressing those sequences in a suitably transformed host include, but are not limited to, using a PCR-assisted mutagenesis technique. Mutations that consist of deletions or additions of amino acid residues to an IL-2 polypeptide can also be made with standard recombinant techniques. In the event of a deletion or addition, the nucleic acid molecule encoding IL-2 is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

The complete amino acid sequence can be used to construct a back-translated gene. A DNA oligomer containing a nucleotide sequence coding for IL-2, IL-4, or IL-13 mutein molecule can be synthesized. For example, several small oligonucleotides coding for portions of the desired polypeptide can be synthesized and then ligated. The individual oligonucleotides typically contain 5′ or 3′ overhangs for complementary assembly.

In addition to generating mutant polypeptides via expression of nucleic acid molecules that have been altered by recombinant molecular biological techniques, subject IL-2, IL-4, or IL-13 mutein molecules can be chemically synthesized. Chemically synthesized polypeptides are routinely generated by those of skill in the art.

Once assembled (by synthesis, site-directed mutagenesis or another method), the DNA sequences encoding an IL-2, IL-4, or IL-13 mutein molecule will be inserted into an expression vector and operatively linked to an expression control sequence appropriate for expression of the IL-2, IL-4, or IL-13 mutein molecule in the desired transformed host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

The DNA sequence encoding the IL-2, IL-4, or IL-13 mutein molecule, whether prepared by site directed mutagenesis, chemical synthesis or other methods, can also include DNA sequences that encode a signal sequence. Such signal sequence, if present, should be one recognized by the cell chosen for expression of the IL-2, IL-4, or IL-13 mutein molecule. It can be prokaryotic, eukaryotic or a combination of the two. It can also be the signal sequence of native IL-2. The inclusion of a signal sequence depends on whether it is desired to secrete the IL-2, IL-4, or IL-13 mutein molecule from the recombinant cells in which it is made. If the chosen cells are prokaryotic, it generally is preferred that the DNA sequence not encode a signal sequence. If the chosen cells are eukaryotic, it generally is preferred that a signal sequence be encoded and most preferably that the wild-type IL-2 signal sequence be used.

E. Nucleic Acid Molecules Encoding Mutant IL-2, IL-4, and/or IL-13

In some embodiments the subject IL-2, IL-4, or IL-13 mutein, either alone or as a part of a molecule (including a IL-2 cytokine fusion), such as those described above, can be obtained by expression of a nucleic acid molecule. Just as IL-2, IL-4, or IL-13 mutein molecules can be described in terms of their identity with wild-type IL-2 polypeptides, the nucleic acid molecules encoding them will necessarily have a certain identity with those that encode wild-type IL-2. For example, the nucleic acid molecule encoding a subject IL-2 mutein can be at least 50%, at least 65%, preferably at least 75%, more preferably at least 85%, and most preferably at least 95% (e.g., 99%) identical to the nucleic acid encoding wild-type IL-2.

In some embodiments the subject IL-2 mutein, either 4 alone or as a part of a chimeric polypeptide (including a IL-4 cytokine fusion), such as those described above, can be obtained by expression of a nucleic acid molecule. Just as IL-4 muteins can be described in terms of their identity with wild-type IL-4 polypeptides, the nucleic acid molecules encoding them will necessarily have a certain identity with those that encode wild-type IL-4. For example, the nucleic acid molecule encoding a subject IL-4 mutein can be at least 50%, at least 65%, preferably at least 75%, more preferably at least 85%, and most preferably at least 95% (e.g., 99%) identical to the nucleic acid encoding wild-type IL-4.

In some embodiments the subject IL-13 mutein, either alone or as a part of a chimeric polypeptide (including a IL-13 cytokine fusion), such as those described above, can be obtained by expression of a nucleic acid molecule. Just as IL-13 muteins can be described in terms of their identity with wild-type IL-13 polypeptides, the nucleic acid molecules encoding them will necessarily have a certain identity with those that encode wild-type IL-13. For example, the nucleic acid molecule encoding a subject IL-13 mutein can be at least 50%, at least 65%, preferably at least 75%, more preferably at least 85%, and most preferably at least 95% (e.g., 99%) identical to the nucleic acid encoding wild-type IL-13.

The nucleic acid molecules provided can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

The nucleic acid molecules are not limited to sequences that encode polypeptides; some or all of the non-coding sequences that lie upstream or downstream from a coding sequence (e.g., the coding sequence of IL-2, IL-4, or IL-13) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary isolated nucleic acid molecules of the present disclosure can include fragments not found as such in the natural state. Thus, this disclosure encompasses recombinant molecules, such as those in which a nucleic acid sequence (for example, a sequence encoding a mutant IL-2, IL-4, or IL-13) is incorporated into a vector (e.g., a plasmid or viral vector) or into the genome of a heterologous cell (or the genome of a homologous cell, at a position other than the natural chromosomal location).

As described above, the subject IL-2, IL-4, or IL-13 mutein molecule may exist as a part of a chimeric polypeptide. In addition to, or in place of, the heterologous polypeptides described above, a subject nucleic acid molecule can contain sequences encoding a “marker” or “reporter.” Examples of marker or reporter genes include R-lactamase, chloramphenicol acetyltransferase (CAT), adenosine deaminase (ADA), aminoglycoside phosphotransferase (neo^{r}, G418^{r}), dihydrofolate reductase (DHFR), hygromycin-β-hosphotransferase (HPH), thymidine kinase (TK), lacz (encoding β-galactosidase), and xanthine guanine phosphoribosyltransferase (XGPRT). One of skill in the art will be aware of additional useful reagents, for example, of additional sequences that can serve the function of a marker or reporter.

The subject nucleic acid molecules can be obtained by introducing a mutation into IL-2-encoding DNA obtained from any biological cell, such as the cell of a mammal. Thus, the subject nucleic acids (and the polypeptides they encode) can be those of a mouse, rat, guinea pig, cow, sheep, horse, pig, rabbit, monkey, baboon, dog, or cat. In one embodiment, the nucleic acid molecules will be those of a human.

### F. Expression of Mutant IL-2, IL-4, OR IL-13 Gene Products

The nucleic acid molecules described above can be contained within a vector that is capable of directing their expression in, for example, a cell that has been transduced with the vector. Accordingly, in addition to the subject IL-2, IL-4, or IL-13 muteins, expression vectors containing a nucleic acid molecule encoding a subject IL-2, IL-4, or IL-13 mutein and cells transfected with these vectors are among the preferred embodiments.

It should of course be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered. For example, vectors that can be used include those that allow the DNA encoding the IL-2, IL-4, or IL-13 muteins to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction of a Modular Dihydrafolate Reductase cDNA Gene: Analysis of Signals Utilized for Efficient Expression”, Mol. Cell. Biol., 2, pp. 1304-19 (1982)) or glutamine synthetase (“GS”) amplification (see, e.g., U.S. Pat. No. 5,122,464 and European published application 338,841).

In some embodiments, the human IL-2, IL-4, or IL-13 muteins of the present disclosure will be expressed from vectors, preferably expression vectors. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are included also.

Exemplary recombinant expression vectors can include one or more regulatory sequences, selected on the basis of the host cells to be used for expression, operably linked to the nucleic acid sequence to be expressed.

The expression constructs or vectors can be designed for expression of an IL-2, IL-4, or IL-13 mutein or variant thereof in prokaryotic or eukaryotic host cells.

Vector DNA can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals.

Expression of proteins in prokaryotes is most often carried out in Escherichia coli with vectors containing constitutive or inducible promoters. Strategies to maximize recombinant protein expression in E. coli can be found, for example, in Gottesman (1990) in Gene Expression Technology: Methods in Enzymology 185 (Academic Press, San Diego, Calif.), pp. 119-128 and Wada et al. (1992) Nucleic Acids Res. 20:2111-2118. Processes for growing, harvesting, disrupting, or extracting the IL-2 mutein or variant thereof from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

In some embodiments the recombinant IL-2, IL-4, or IL-13 muteins or biologically active variants thereof can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerenvisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2^{nd} ed., Cold Spring Harbor Laboratory Press, Plainview, N.Y.). See, Goeddel (1990) in Gene Expression Technology: Methods in Enzymology 185 (Academic Press, San Diego, Calif.).

The sequences encoding the human IL-2, IL-4, or IL-13 mutein of the present disclosure can be optimized for expression in the host cell of interest. The G-C content of the sequence can be adjusted to levels average for a given cellular host, as calculated by reference to known genes expressed in the host cell. Methods for codon optimization are well known in the art. Codons within the IL-2, IL-4, or IL-13 mutein coding sequence can be optimized to enhance expression in the host cell, such that about 1%, about 5%, about 10%, about 25%, about 50%, about 75%, or up to 100% of the codons within the coding sequence have been optimized for expression in a particular host cell.

Vectors suitable for use include T7-based vectors for use in bacteria (see, for example, Rosenberg et al., Gene 56:125, 1987), the pMSXND expression vector for use in mammalian cells (Lee and Nathans, J. Biol. Chem. 263:3521, 1988), and baculovirus-derived vectors (for example, the expression vector pBacPAK9 from Clontech, Palo Alto, Calif.) for use in insect cells.

In some embodiments nucleic acid inserts, which encode the subject IL-2, IL-4, or IL-13 mutein in such vectors, can be operably linked to a promoter, which is selected based on, for example, the cell type in which expression is sought.

In selecting an expression control sequence, a variety of factors should also be considered. These include, for example, the relative strength of the sequence, its controllability, and its compatibility with the actual DNA sequence encoding the subject IL-2, IL-4, or IL-13 mutein, particularly as regards potential secondary structures. Hosts should be selected by consideration of their compatibility with the chosen vector, the toxicity of the product coded for by the DNA sequences of this invention, their secretion characteristics, their ability to fold the polypeptides correctly, their fermentation or culture requirements, and the ease of purification of the products coded for by the DNA sequences.

Within these parameters one of skill in the art may select various vector/expression control sequence/host combinations that will express the desired DNA sequences on fermentation or in large scale animal culture, for example, using CHO cells or COS 7 cells.

The choice of expression control sequence and expression vector, in some embodiments, will depend upon the choice of host. A wide variety of expression host/vector combinations can be employed. Useful expression vectors for eukaryotic hosts, include, for example, vectors with expression control sequences from SV40, bovine papilloma virus, adenovirus and cytomegalovirus. Useful expression vectors for bacterial hosts include known bacterial plasmids, such as plasmids from E. co/i, including col El, pCRI, pER32z, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Useful expression vectors for yeast cells include the 2μ plasmid and derivatives thereof. Useful vectors for insect cells include pVL 941 and pFastBac™ 1 (GibcoBRL, Gaithersburg, Md.). Cate et al., “Isolation Of The Bovine And Human Genes For Mullerian Inhibiting Substance And Expression Of The Human Gene In Animal Cells”, Cell, 45, pp. 685-98 (1986).

In addition, any of a wide variety of expression control sequences can be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, for example PL, the control regions of fd coat protein, the promoter for 3-phosphoglycerate kinase or other glycolytic enzymes, the promoters of acid phosphatase, e.g., PhoA, the promoters of the yeast a-mating system, the polyhedron promoter of Baculovirus, and other sequences known to control the expression of genes of prokaryotic or eukaryotic cells or their viruses, and various combinations thereof.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neo^{r}) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the invention include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a subject IL-2 mutein disclosed herein are also features of the invention. A cell of the invention is a transfected cell, i.e., a cell into which a nucleic acid molecule, for example a nucleic acid molecule encoding a IL-2, IL-4, or IL-13 mutein, has been introduced by means of recombinant DNA techniques. The progeny of such a cell are also considered within the scope of the invention.

The precise components of the expression system are not critical. For example, an IL-2 mutein can be produced in a prokaryotic host, such as the bacterium E. coli, or in a eukaryotic host, such as an insect cell (e.g., an Sf21 cell), or mammalian cells (e.g., CHO, HEK293, COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

In some embodiments, IL-2, IL-4, or IL-13 mutein obtained will be glycosylated or unglycosylated depending on the host organism used to produce the mutein. If bacteria are chosen as the host then the IL-2, IL-4, or IL-13 mutein produced will be unglycosylated. Eukaryotic cells, on the other hand, will glycosylate the IL-2, IL-4, or IL-13 muteins s, although perhaps not in the same way as native-IL-2 is glycosylated. The IL-2, IL-4, or IL-13 mutein produced by the transformed host can be purified according to any suitable method. Various methods are known for purifying IL-2, IL-4, or IL-13 mutein. See, e.g., Current Protocols in Protein Science, Vol 2. Eds: John E. Coligan, Ben M. Dunn, Hidde L. Ploehg, David W. Speicher, Paul T. Wingfield, Unit 6.5 (Copyright 1997, John Wiley and Sons, Inc. IL-2, IL-4, or IL-13 muteins can be isolated from inclusion bodies generated in E. coli, or from conditioned medium from either mammalian or yeast cultures producing a given mutein using cation exchange, gel filtration, and/or reverse phase liquid chromatography.

Another exemplary method of constructing a DNA sequence encoding the IL-2, IL-4, or IL-13 muteins s is by chemical synthesis. This includes direct synthesis of a peptide by chemical means of the protein sequence encoding for an IL-2 mutein exhibiting the properties described. This method can incorporate both natural and unnatural amino acids at positions that affect the interactions of IL-2, IL-4, or IL-13 mutein with the their corresponding receptors. Alternatively a gene which encodes the desired IL-2, IL-4, or IL-13 mutein can be synthesized by chemical means using an oligonucleotide synthesizer. Such oligonucleotides are designed based on the amino acid sequence of the desired IL-2, IL-4, or IL-13 mutein, and preferably selecting those codons that are favored in the host cell in which the recombinant mutein will be produced. In this regard, it is well recognized that the genetic code is degenerate—that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated that for a given DNA sequence encoding a particular IL-2 mutein, there will be many DNA degenerate sequences that will code for that IL-2 mutein. For example, it will be appreciated that in addition to the preferred DNA sequence for mutein H9, there will be many degenerate DNA sequences that code for the IL-2, IL-4, or IL-13 mutein shown. These degenerate DNA sequences are considered within the scope of this disclosure. Therefore, “degenerate variants thereof in the context of this invention means all DNA sequences that code for and thereby enable expression of a particular mutein.

The biological activity of the IL-2, IL-4, or IL-13 muteins s can be assayed by any suitable method known in the art. Such assays include PHA-blast proliferation and NK cell proliferation.

### G. Anti-PD-1 Antibodies and Combinations

In some embodiments, the immunomodulatory agent administered with the IL-4R or IL-13 receptor targeted cargo protein includes an anti-PD-1 antibody. Anti-PD-1 antibodies for use according to the invention and methods described herein include but are not limited to nivolumab, BMS-936558, MDX-1106, ONO-4538, AMP224, CT-011, and MK-3475 (pembrolizumab), cemiplimab (REGN2810), SHR-1210 (CTR20160175 and CTR20170090), SHR-1210 (CTR20170299 and CTR20170322), JS-001 (CTR20160274), IBI308 (CTR20160735), BGB-A317 (CTR20160872) and/or a PD-1 antibody as recited in U.S. Patent Publication No. 2017/0081409. There are two approved anti-PD-1 antibodies, pembrolizumab (Keytruda®; MK-3475-033) and nivolumab (Opdivo®; CheckMate078) and many more in development which can be used in combination described herein. In some embodiments, the IL-2 mutein used in combination with an anti-PD-1 antibody is a fusion mutein as described herein.

### H. Anti-PD-L1 Antibodies and Combinations

In some embodiments, the immunomodulatory agent administered with the IL-4R or IL-13 receptor targeted cargo protein includes an anti-PD-L1 antibody. There are three approved anti-PD-L1 antibodies, atezolizumab (TECENTRIQ®; MPDL3280A), avelumab (BAVENCIO®; MSB0010718C), and Durvalumab (MEDI4736), as well as other anti-PD-L1 antibodies in development. Numerous anti-PD-L1 antibodies are available and many more in development which can be used in combination with the IL-2 muteins as described herein. In some embodiments, the PD-L1 antibody is one described in U.S. Patent Publication No. 2017/0281764 as well as International Patent Publication No. WO 2013/079174 (avelumab) and WO 2010/077634 (or U.S. Patent Application No. 20160222117 or U.S. Pat. No. 8,217,149; atezolizumab). In some embodiments, the PD-L1 antibody comprises a heavy chain sequence of SEQ ID NO:34 and a light chain sequence of SEQ ID NO:36 (from US 2017/281764). In some embodiments, the PD-L1 antibody is atezolizumab (TECENTRIQ®; MPDL3280A; iMpower110). In some embodiments, the PD-L1 antibody is avelumab (BAVENCIO®; MSB0010718C). In some embodiments, the PD-L1 antibody is durvalumab (MEDI4736). In some embodiments, the PD-L1 antibody includes, for example, Atezolizumab (iMpower133), BMS-936559/MDX-1105, and/or RG-7446/MPDL3280A, and/or YW243.55.S70, as well as any of the exemplary anti-PD-L1 antibodies provided herein.

In some embodiments, the IL-2 mutein comprising substitutions L80F, R81D, L85V, 186V, and 192F, numbered in accordance with human wild-type IL-2 is used in combination with any of the referenced antibodies. In some embodiments, the IL-2 mutein further comprises F42A substitution, wherein numbering is in accordance with the wild-type human IL-2. In some embodiments, the IL-2 mutein further comprises Y45A substitution, wherein numbering is in accordance with the wild-type human IL-2. In some embodiments, the IL-2 mutein further comprises E62A substitution, wherein numbering is in accordance with the wild-type human IL-2. In some embodiments, the IL-2 mutein used and/or fusion in combination with an anti-PD-L1 antibody is a fusion mutein as described herein. In some embodiments, the IL-2/IL-13 mutein used in combination with an anti-PD-L1 antibody is a fusion mutein as described herein.

### I. Other Immunotherapy Combinations

Other antibodies for use with the subject compositions and methods provided herein include but are not limited to, anti-PD-1 antibodies such as nivolumab (OPDIVO®), BMS-936558, MDX-1106, ONO-4538, AMP224, CT-011, and MK-3475 (pembrolizumab or KEYTRUDA®), cemiplimab (REGN2810), SHR-1210 (CTR20160175 and CTR20170090), SHR-1210 (CTR20170299 and CTR20170322), JS-001 (CTR20160274), IBI308 (CTR20160735), BGB-A317 (CTR20160872), anti-PD-L1 antagonistic antibodies such as BMS-936559/MDX-1105, MEDI4736, and RG-7446/MPDL3280A. In some embodiments, the IL-2 mutein comprising substitutions L80F, R81D, L85V, 186V, and 192F, numbered in accordance with human wild-type IL-2 is used in combination with any of the referenced antibodies. In some embodiments, the IL-2 mutein further comprises F42A substitution, wherein numbering is in accordance with the wild-type human IL-2. In some embodiments, the IL-2 mutein further comprises Y45A substitution, wherein numbering is in accordance with the wild-type human IL-2. In some embodiments, the IL-2 mutein further comprises E62A substitution, wherein numbering is in accordance with the wild-type human IL-2. In some embodiments, the IL-2 mutein further comprises E62A substitution, wherein numbering is in accordance with the wild-type human IL-2. In some embodiments, the IL-2 mutein is any IL-2 mutein or variant disclosed herein. In some embodiments, the IL-2 mutein sequence is 90% identical to any one of the wild type or any of the SEQ ID Nos provided in Table 2..

Other antibodies can also include monoclonal antibodies to solid tumors as well as sarcoma, carcinoma, head and neck cancer, glioblastoma, bladder cancer, oral cancer, mesothelioma, pancreatic cancer, liver cancer, colorectal cancer, pulmonary cancer, cutaneous, lymphoid, gastrointestinal cancer, prostate cancer, ovarian cancer, breast cancer, basal-like breast tumor, endometrial cancer, multiple myeloma, melanoma, lymphoma, lung cancer (including small cell lung cancer), kidney cancer, gastric cancer, brain cancer, and CNS tumors (see, generally www.clinicaltrials.gov). CNS tumors include glioma, glioblastoma, glioblastoma multiforme (GBM), refractory glioblastoma multiforme (rGBM), recurrent glioblastoma, astrocytoma, medulloblastoma, craniopharyogioma, ependymoma, pinealoma, hemangioblastoma, acoustic neuroma, oligodendroglia, menangioma, meningioma, neuroblastoma, retinoblastoma, medulloblastoma, adult pituitary adenoma, an 06-methylguanine-methyltransferase (MGMT) positive or negative CNS tumor, and furin positive CNS tumor.

In some embodiments, antibodies can also include antibodies for antibody-dependent cell-mediated cytotoxicity (ADCC).

### J. Methods of Treatment

The IL-4 receptor targeted cargo protein or IL-13 receptor targeted cargo protein can be administered to a subject in combination with an immunomodulatory agent (e.g., an IL-2 mutein) to treat a disorder associated with abnormal apoptosis or a differentiative process (e.g., cellular proliferative disorders or cellular differentiative disorders, such as cancer, by, for example, producing an active or passive immunity). In some embodiments, the disorder is a cancer that is associated with IL-4R or Il-13Rα2 overexpression.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of sarcoma, carcinoma, head and neck cancer, glioblastoma, bladder cancer, oral cancer, mesothelioma, pancreatic cancer, liver cancer, colorectal cancer, pulmonary cancer, cutaneous, lymphoid, gastrointestinal cancer, prostate cancer, ovarian cancer, breast cancer, basal-like breast tumor, endometrial cancer, multiple myeloma, melanoma, lymphoma, lung cancer (including small cell lung cancer), kidney cancer, gastric cancer, brain cancer, and CNS tumors. In some embodiments, the cancer for treatment is a solid tumor. In some embodiments, the cancer for treatment includes but is not limited to sarcoma, carcinoma, head and neck cancer, glioblastoma, bladder cancer, oral cancer, mesothelioma, pancreatic cancer, liver cancer, colorectal cancer, pulmonary cancer, cutaneous, lymphoid, gastrointestinal cancer, prostate cancer, ovarian cancer, breast cancer, basal-like breast tumor, endometrial cancer, multiple myeloma, melanoma, lymphoma, lung cancer (including small cell lung cancer), kidney cancer, gastric cancer, brain cancer, and CNS tumors. CNS tumors include glioma, glioblastoma, glioblastoma multiforme (GBM), refractory glioblastoma multiforme (rGBM), recurrent glioblastoma, astrocytoma, medulloblastoma, craniopharyogioma, ependymoma, pinealoma, hemangioblastoma, acoustic neuroma, oligodendroglia, menangioma, meningioma, neuroblastoma, retinoblastoma, medulloblastoma, adult pituitary adenoma, an 06-methylguanine-methyltransferase (MGMT) positive or negative CNS tumor, and furin positive CNS tumor.

The IL-4 receptor targeted cargo protein or IL-13 receptor targeted cargo protein/immunomodulatory agent (e.g., an IL-2 mutein) combination therapy can be used to treat patients who have, who are suspected of having, or who may be at high risk for developing any type of cancer, including renal carcinoma or melanoma, or any viral disease, including for example human papillomavirus (HPV) and/or Hepatitis, such as Hepatitis A, Hepatitis B, Hepatitis C, and/or Hepatitis D. Exemplary carcinomas include those forming from tissue of the cervix, lung, prostate, breast, head and neck, colon and ovary. The term also includes carcinosarcomas, which include malignant tumors composed of carcinomatous and sarcomatous tissues.

Additional examples of proliferative disorders include hematopoietic neoplastic disorders.

Alternatively, or in addition to methods of direct administration to patients, in some embodiments, IL-4 receptor targeted cargo protein or IL-13 receptor targeted cargo protein/immunomodulatory agent (e.g. IL-2 mutein) combinations can be used in ex vivo methods. For example, cells (e.g., peripheral blood lymphocytes or purified populations of lymphocytes isolated from a patient and placed or maintained in culture) can be cultured in vitro in culture medium and the contacting step can be affected by adding the IL-2 mutant to the culture medium. The culture step can include further steps in which the cells are stimulated or treated with other agents, e.g., to stimulate proliferation, or to expand a population of cells that is reactive to an antigen of interest (e.g., a cancer antigen or a viral antigen). The cells are then administered to the patient after they have been treated.

In some embodiments, the IL-4 receptor targeted cargo protein or IL-13 receptor targeted cargo protein is administered prior to the immunomodulatory agent. In some embodiments, the IL-4 receptor targeted cargo protein or IL-13 receptor targeted cargo protein is administered after administering the immunomodulatory agent. In some embodiments, the IL-4 receptor targeted cargo protein or IL-13 receptor targeted cargo protein is administered contemporaneously with the immunomodulatory agent.

Anti-PD-1 antibodies for use as an immunomodulatory agent in combination with the IL-4 receptor targeted cargo protein or IL-13 receptor targeted cargo protein disclosed herein for the cancer and/or proliferative disorder treatment methods include but are not limited to nivolumab (OPDIVO®), BMS-936558, MDX-1106, ONO-4538, AMP224, CT-011, and MK-3475 (pembrolizumab or KEYTRUDA®), cemiplimab (REGN2810), SHR-1210 (CTR20160175 and CTR20170090), SHR-1210 (CTR20170299 and CTR20170322), JS-001 (CTR20160274), IB1308 (CTR20160735), BGB-A317 (CTR20160872).

In some embodiments, the IL-4 receptor targeted cargo protein or IL-13 receptor targeted cargo protein as described herein is used in combination with antibodies including dupilumab, nivolumab (OPDIVO®), BMS-936558, MDX-1106, ONO-4538, AMP224, CT-011, and MK-3475 (pembrolizumab or KEYTRUDA®), cemiplimab (REGN2810), SHR-1210 (CTR20160175 and CTR20170090), SHR-1210 (CTR20170299 and CTR20170322), JS-001 (CTR20160274), IBI308 (CTR20160735), BGB-A317 (CTR20160872). In some embodiments, the IL-2, IL-4, or IL-13 mutein and/as described herein is used in combination with antibodies including anti-PD-L1 antagonistic antibodies such as BMS-936559/MDX-1105, MEDI4736, and RG-7446/MPDL3280A.

In some embodiments, the IL-2, IL-4, or IL-13 mutein and/as described herein is fused with an antibody selected from dupilumab, nivolumab (OPDIVO®), BMS-936558, MDX-1106, ONO-4538, AMP224, CT-011, and MK-3475 (pembrolizumab or KEYTRUDA®), cemiplimab (REGN2810), SHR-1210 (CTR20160175 and CTR20170090), SHR-1210 (CTR20170299 and CTR20170322), JS-001 (CTR20160274), IBI308 (CTR20160735), BGB-A317 (CTR20160872). In some embodiments, the IL-2, IL-4, or IL-13 mutein and/as described herein is fused with an antibody including but not limited to anti-PD-L1 antagonistic antibodies such as BMS-936559/MDX-1105, MEDI4736, and RG-7446/MPDL3280A.

### K. Pharmaceutical Compositions and Methods of Administration

In some embodiments, subject IL-4 receptor targeted cargo moieties or IL-13 receptor targeted cargo moieties can be incorporated into compositions with an immunomodulatory agent (e.g., IL-2 mutein), including pharmaceutical compositions. Such compositions typically include a) a IL-4 receptor targeted cargo protein or IL-13 receptor targeted cargo protein, b) an immunomodulatory agent (e.g., an IL-2 mutein) and a pharmaceutically acceptable carrier. Such compositions can also comprise anti-PD-1 antibodies.

The a) IL-4 receptor targeted cargo protein or IL-13 receptor targeted cargo protein, and b) an immunomodulatory agent (e.g., an IL-2 mutein) can be administered as a co-composition, simultaneously as two separate compositions, and/or sequentially as two separate compositions. In some embodiments, the a) IL-4 receptor targeted cargo protein or IL-13 receptor targeted cargo protein, and b) immunomodulatory agent (e.g., an IL-2 mutein) are administered together as a single co-composition (i.e., co-formulated). In some embodiments, the a) IL-4 receptor targeted cargo protein or IL-13 receptor targeted cargo protein, and b) immunomodulatory agent (e.g., an IL-2 mutein) are administered simultaneously as two separate compositions (i.e., separate formulations). In some embodiments, the a) IL-4 receptor targeted cargo protein or IL-13 receptor targeted cargo protein, and b) immunomodulatory agent (e.g., an IL-2 mutein) are administered sequentially as separate compositions (i.e., separate formulations). In some embodiments, when the a) IL-4 receptor targeted cargo protein or IL-13 receptor targeted cargo protein, and b) immunomodulatory agent (e.g., an IL-2 mutein) are administered sequentially as separate compositions, the IL-4 receptor targeted cargo protein or IL-13 receptor targeted cargo protein is administered before the immunomodulatory agent. In some embodiments, when the a) IL-4 receptor targeted cargo protein or IL-13 receptor targeted cargo protein and b) immunomodulatory agent are administered sequentially as separate compositions, the immunomodulatory agent (e.g., an IL-2 mutein) is administered before the IL-4 receptor targeted cargo protein or IL-13 receptor targeted cargo protein. In some embodiments, the IL-4 receptor targeted cargo protein is SEQ ID NO:1057. In some embodiments, the IL-13 receptor targeted cargo protein comprises an IL-13 mutein having the amino acid sequence of SEQ ID NO:106. In some embodiments, the cargo moiety is Pseudomonas exotoxin (PE). In some embodiments, the immunomodulatory agent is an IL-2 mutein fusion. In some embodiments, the IL-2 mutein fusion has the amino acid sequence of SEQ ID NO: 31.

A pharmaceutical composition is formulated to be compatible with its intended route of administration. The IL-4 receptor targeted cargo protein or IL-13 receptor targeted cargo protein and b) immunomodulatory agent of the invention may be given orally, but it is more likely that they will be administered through a parenteral route, including for example intravenous administration. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, the IL-4 receptor targeted cargo protein or IL-13 receptor targeted cargo protein, and immunomodulatory agent, are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the IL-4 receptor targeted cargo protein or IL-13 receptor targeted cargo protein, and immunomodulatory agent can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, compounds (the IL-4 receptor targeted cargo protein or IL-13 receptor targeted cargo protein, and immunomodulatory agent) can also be prepared in the form of suppositories (e.g., with conventional suppository bases such as cocoa butter and other glycerides) or retention enemas for rectal delivery.

In some embodiments, compounds (the IL-4 receptor targeted cargo protein or IL-13 receptor targeted cargo protein, and immunomodulatory agent) can also be administered by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996).

In one embodiment, the IL-4 receptor targeted cargo protein or IL-13 receptor targeted cargo protein, and immunomodulatory agent are prepared with carriers that will protect the composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Dosage, toxicity and therapeutic efficacy of such IL-4 receptor targeted cargo protein or IL-13 receptor targeted cargo protein, and immunomodulatory agent can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, single dose amounts of the IL-4 receptor targeted cargo protein or IL-13 receptor targeted cargo protein, and/or immunomodulatory agent can be in the range of approximately 0.001 mg/kg to 0.1 mg/kg of patient body weight can be administered. In some embodiments, single dose amounts of the anti-PD-1 antibody or inhibitor can be in the range of approximately 1 mg/kg to 20 mg/kg, or about 5 mg/kg to about 15 mg/kg, or about 10 mg/kg of patient body weight can be administered. In some embodiments, doses of the anti-PD-1 antibody or inhibitor and/or the IL-2 mutein of about 0.005 mg/kg, 0.01 mg/kg, 0.025 mg/kg, 0.05 mg/kg, 0.1 mg/kg, 0.25 mg/kg, 0.5 mg/kg, 1.0 mg/kg, 5.0 mg/kg, 10.0 mg/kg may be administered. In some embodiments, 600,000 IU/kg is administered (IU can be determined by a lymphocyte proliferation bioassay and is expressed in International Units (IU) as established by the World Health Organization 1^{st} International Standard for Interleukin-2 (human)). The dosage may be similar to, but is expected to be less than, that prescribed for PROLEUKIN®. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the subject IL-2 muteins can include a single treatment or, can include a series of treatments. In one embodiment, the compositions are administered every 8 hours for five days, followed by a rest period of 2 to 14 days, e.g., 9 days, followed by an additional five days of administration every 8 hours. In some embodiments, administration is 3 doses administered every 4 days.

The pharmaceutical compositions can be included in a container, pack, or dispenser together with instructions for administration.

### L. Alternative Embodiments

Provided herein are methods of treating a cancer comprising administering: a) an IL-4 targeted cargo moiety or an IL-13 targeted cargo moiety; and b) immunomodulatory agent (e.g., an IL-2 mutein described herein). Such combination therapies are particularly useful for the treatment of cancers that overexpress IL-4R or IL-13Rα2. Also provided herein are composition for use with the subject methods. Aspects of the methods and compositions are further detailed below.

In one aspect, provided herein is a method of treating a cancer in a patient in need thereof comprising administering to the patient: a) an IL-4 targeted cargo moiety or an IL-13 targeted cargo moiety; and b) an immunomodulatory agent, wherein the IL-4 targeted cargo moiety comprises an IL-4 or an IL-4 mutein linked to a cargo moiety, wherein the IL-13 targeted cargo moiety comprises an IL-13 or an IL-13 mutein linked to a cargo moiety, and wherein the cargo moiety is capable of inhibiting cancer cell growth.

In some embodiments, the IL-4 or IL-4 mutein is selected from the IL-4 muteins in Table 6. In some embodiments, the IL-4 targeted cargo moiety has the amino acid sequence of SEQ ID NO:1057.

In some embodiments, the IL-13 or IL-13 mutein is selected from the IL-13 muteins disclosed in Tables 5, 7, and 8. In some embodiments, the IL-13 mutein has the amino acid sequence of SEQ ID NO:106.

In some embodiments, the immunomodulatory agent is an IL-2 mutein or IL-2 mutein fusion protein. In some embodiments, the IL-2 mutein or IL-2 mutein fusion protein is selected from IL-2 mutein or IL-2 mutein fusion protein in Tables 2-4. In some embodiments, the IL-2 mutein fusion protein has the amino acid of SEQ ID NO:31.

In some embodiments, the cargo moiety is selected from Pseudomonas exotoxin, Aerolysin, Proaerolysin, Bouganin, a Cholera toxin, and Ribonuclease A. In some embodiments, the cargo moiety is Pseudomonas exotoxin.

In some embodiments, the cancer is associated with IL-4R overexpression or IL-13Rα2 overexpression. In some embodiments, the cancer is refractory to immune-checkpoint inhibition.

In some embodiments, the cancer is selected from the group consisting of sarcoma, carcinoma, head and neck cancer, glioblastoma, bladder cancer, oral cancer, mesothelioma, pancreatic cancer, liver cancer, colorectal cancer, pulmonary cancer, cutaneous, lymphoid, gastrointestinal cancer, prostate cancer, ovarian cancer, breast cancer, basal-like breast tumor, endometrial cancer, multiple myeloma, melanoma, lymphoma, lung cancer (including small cell lung cancer), kidney cancer, gastric cancer, brain cancer, and CNS tumors.

In some embodiments, the IL-4 targeted cargo moiety or an IL-13 targeted cargo moiety is administered before the immunomodulatory agent. In certain embodiments, the IL-4 targeted cargo moiety or an IL-13 targeted cargo moiety is administered after the immunomodulatory agent. In some embodiments, the IL-4 targeted cargo moiety or an IL-13 targeted cargo moiety is administered contemporaneously with the immunomodulatory agent.

In another aspect, provided herein is a composition comprising: a) an IL-4 targeted cargo moiety or an IL-13 targeted cargo moiety; and b) an immunomodulatory agent, wherein the IL-4 targeted cargo moiety comprises an IL-4 or an IL-4 mutein linked to a cargo moiety, wherein the IL-13 targeted cargo moiety comprises an IL-13 or an IL-13 mutein linked to a cargo moiety, and wherein the cargo moiety is capable of inhibiting cancer cell growth.

In some embodiments, the IL-4 or IL-4 mutein is selected from the IL-4 muteins in Table 6. In some embodiments, the IL-4 targeted cargo moiety has the amino acid sequence of SEQ ID NO:1057.

In some embodiments, the IL-13 or IL-13 mutein is selected from the IL-13 muteins disclosed in Tables 5, 7, and 8. In some embodiments, the IL-13 mutein has the amino acid sequence of SEQ ID NO:106.

In some embodiments, the immunomodulatory agent is an IL-2 mutein or IL-2 mutein fusion protein. In some embodiments, the IL-2 mutein or IL-2 mutein fusion protein is selected from IL-2 mutein or IL-2 mutein fusion protein in Tables 2-4. In some embodiments, the IL-2 mutein fusion protein has the amino acid of SEQ ID NO:31.

In some embodiments, the cargo moiety is selected from Pseudomonas exotoxin, Aerolysin, Proaerolysin, Bouganin, a Cholera toxin, and Ribonuclease A. In some embodiments, the cargo moiety is Pseudomonas exotoxin.

The following examples are provided to describe certain embodiments of the invention provided herein and are not to be construed to as limiting.

### EXAMPLES

### Example 1: Evaluation of MDNA132

### Introduction

IL-13 binds to the IL-13Rα2 chain, which does not have an intra-cellular signaling domain and is generally thought to act as a decoy receptor. IL-13Rα2 is expressed in a limited number of normal tissues, with highest transcript levels observed in the testes. In contrast, IL-13Rα2 mRNA is observed in a wide range of tumors (glioblastoma, colorectal, pancreatic, melanoma and basal-like breast tumors) and is associated with poor prognosis and survival. Therefore, IL-13Rα2 is considered as a potential tumor-associated antigen (TAA) that can be targeted to deliver a therapeutic payload to tumors sites (Okamoto H et al., “Interleukin-13 receptor 02 is a novel marker and potential therapeutic target for human melanoma,” Sci Rep (2019) 9: 1281) and avoid causing damage to normal tissues.

MDNA132 is an IL-13 superkine engineered to bind IL-13Rα2 with a 10-fold increased affinity compared to IL-13, and with reduced affinity for functional receptor, IL-13a1 (Moraga et al., “Instructive roles for cytokine-receptor binding parameters in determining signaling and functional potency,” Science Signaling (2015) 8(402): ra114), thereby achieving high selectivity towards the decoy receptor expressed on tumors. Thus, MDNA132 can be used to effectively to deliver therapeutic payloads to the tumors overexpressing the IL-13 decoy receptor.

A long-acting version of MDNA132 (i.e., Fc-MDNA132) was characterized by testing for decoy receptor selectivity and its capability to localize in IL-13Rα2 expressing tumors.

Binding studies with different versions of Fc-MDNA132 constructs (Fc-MDNA132LR, Fc-MDNA132LQ, MDNA132-Fc) showed clear selectivity for IL-13Rα2 over IL-13Rα1. However, Fc-MDNA132 did not have higher binding affinity for IL-13Rα2 than Fc-IL13 as reported (Moraga et al., “Instructive roles for cytokine-receptor binding parameters in determining signaling and functional potency,” Science Signaling (2015) 8(402): ra114). To increase binding affinity to IL-13Rα2, cytokine-receptor structural modelling studies were conducted to identify additional modifications in Fc-MDNA132 that may enhance binding to IL-13Rα2. The testing and characterization of these novel MDNA132 variants are described in this report.

Imaging studies with tumor bearing mice showed accumulation of labeled Fc-MDNA132 in IL-13Rα2 expressing A375 and U87 tumors but not in A549 tumors, which lack L-13Rα2 expression. These results demonstrated the selective accumulation of Fc-MDNA132 in IL-13Rα2 expressing tumors and validate the strategy to design IL-13Rα2 cancer targeting constructs to selectively deliver therapeutic payloads to IL-13Rα2 expressing tumors.

TABLE 10
List of constructs tested so far. The table below captures information on the constructs
that were produced for testing under MDNA132 project. The constructs are fusion
proteins using (G4S)x3 (SEQ ID NO: 1002) as linker unless stated otherwise.

| | | Endotoxin | |
|---|---|---|---|
| Construct | Purity % | (EU/mg) | Comments |
| Fc-MDNA132 (1:1 KIH) | 99.2 | <1.36 | The original MDNA132 (L39) fused to |
| | | | Fc (KIH format) |
| mCD3-MDNA132 (KiH, 1:1) | 100 | <0.05 | Mouse anti-CD3 fused to MDNA132. |
| | | | MTD established |
| huCD3-MDNA132 (KiH, 1:1) | 100 | <0.05 | Human anti-CD3 fused to MDNA132. |
| mPD1-MDNA132 (KiH, 1:1) | 93 | <0.05 | Mouse anti-PD1 fused to MDNA132. |
| huPD1-MDNA132 (KiH, 1:1) | 98 | <0.05 | Human anti-PD1 fused to MDNA132. |
| MDNA132LR-Fc (1:1 KIH) | 100 | <1 | L39 and R111 version of MDNA132 |
| | | | fused to Fc at N terminus. No effect on |
| | | | binding affinity to IL-13Ra2 |
| Fc-MDNA132LR (1:1 KIH) | 100 | <1 | L39 and R111 version of MDNA132 |
| | | | fused to Fc at C terminus. No effect on |
| | | | binding affinity to IL-13Ra2 |
| Fc-MDNA132 (1:2) | 94.7 | <0.05 | L39 version as a homodimer. No |
| | | | effect on binding affinity to IL-13Ra2 |
| Fc-MDNA132.89 (KIH) | 98.8 | <1 | New MDNA132 mutein based on |
| | | | structural modeling. Enhanced |
| | | | binding affinity to IL-13Ra2 not |
| | | | observed |
| Fc-MDNA132.90 (KIH) | 99 | <1 | New MDNA132 mutein based on |
| | | | structural modeling. Enhanced |
| | | | binding affinity to IL-13Ra2 not |
| | | | observed |
| Fc-MDNA132.15 (KIH) | 99.8 | <1 | New MDNA132 mutein based on |
| | | | structural modeling. Enhanced |
| | | | binding affinity to IL-13Ra2 observed |
| | | | by ~8-fold |
| Fc-MDNA132.100 (KIH) | 99.3 | <1 | New MDNA132 mutein based on |
| | | | structural modeling. Enhanced |
| | | | binding affinity to IL-13Ra2 not |
| | | | observed |
| Fc-MDNA132.7 (KIH) | 99.3 | <1 | New MDNA132 mutein based on |
| | | | structural modeling. Enhanced |
| | | | binding affinity to IL-13Ra2 not |
| | | | observed |
| Fc-MDNA132.91R (KIH) | 99.95 | <1 | New MDNA132 mutein based on |
| | | | structural modeling. Enhanced |
| | | | binding affinity to IL-13Ra2 not |
| | | | observed |
| Fc^{act}-MDNA132.15 (KIH | 99.1 | <1 | MDNA132.15 (mew variant) fused to |
| 1:1) | | | Fc with effector function |
| Mouse anti-PD1- | 95.8 | <1 | Mouse anti-PD1 fused to |
| MDNA132.15 | | | MDNA132.15 |

### Study 1: Binding Affinity of Fc-MDNA132 to Mouse and Cyno IL-13 Receptors

The objective of the study was to test the binding affinity of Fc-MDNA132 (1:1 KIH) to mouse and cynomolgus IL-13 receptors to identify appropriate animal models for efficacy and toxicology studies.

### Methodology

Receptor binding was assessed using surface plasmon resonance (SPR) in which test constructs (i.e., Fc-MDNA132) were bound onto SPR chip surface and receptors (i.e., IL-13Rα1 and IL-13Rα2) were used as flow analytes (data not shown).

### Results

Fc-MDNA132 (1:1 KIH) was tested for binding to mouse and cynomolgus IL-13Rα1 and to mouse IL-13Rα2 since cynomolgus homologue of IL-13Rα2 is not commercially available. There was no binding to both mouse and cynomolgus IL-13Rα1 (data not shown). Fc-MDNA132 bound mouse IL-13Rα2 with a K_{D} of 3.1 nM, similar to its binding affinity to human IL-13Rα2 (K_{D} of 2.5 nM).

### Conclusion

Fc-MDNA132 (1:1 KIH) binds mouse IL-13Rα2 (K_{D}=3.1 nM) with similar affinity as to human (K_{D}=2.5 nM). There was no binding to IL-13Rα1 of mouse, cynomolgus and human.

### Study 2: Testing of MDNA132 Variants Designed Based on Cytokine-Receptor Structural Modeling

Several MDNA132 variants (Table 11) were designed based on structural modeling of the cytokine-receptor complex with the objective of increasing affinity for IL-13Rα2 while maintaining selectivity (i.e., no binding to IL-13Rα1).

TABLE 11
MDNA132 constructs

| Construct | Description |
|---|---|
| Fc-IL13 (1:1 KIH) | One molecule of human wild-type IL-13 (hIL-13) fused to 1 Fc |
| | (hIgG1 with N297A mutation) at C-terminus using a (G4S)3 |
| | (SEQ ID NO: 1002) linker. |
| | Mutation in Fc suppresses effector function of Fc. |
| Fc-MDNA132.89 (1:1 KIH) | Fc heterodimer in KIH format containing 1 MDNA132 variants |
| Fc-MDNA132.90 (1:1 KIH) | (single mutants) fused to 1 Fc (hIgG1 with N297A mutation) at |
| Fc-MDNA132.15 (1:1 KIH) | N-terminus using a (G4S)3 (SEQ ID NO: 1002) linker. |
| Fc-MDNA132.100 (1:1 KIH) | Mutation in Fc suppresses effector function of Fc. |
| Fc-MDNA132.7 (1:1 KIH) | |
| Fc-MDNA132.91R (1:1 KIH) | |

### Methodology

MDNA132 variants (Table 11) were assessed for binding to IL-13Rα1 and IL-13Rα2 by SPR.

### Results

Representative SPR sensorgrams of Fc-MDNA132 and Fc-51.13 binding to IL-3Rα1 and IL-13Rα2 is shown in (data not shown) and K_{D} values are tabulated in Table 12. Variants contain the 5 core mutations of MDNA132 and an added mutation at amino acid position indicated in the name of the constructs. All Fc-MDNA132 variants tested did not bind to IL-13αR1 and only Fc-MDNA132.15 exhibited higher affinity for IL-13Rα2 than parental or original Fc-MDNA132.

TABLE 12
Binding KD (nM) values for various constructs
to human IL-13Rα1 and IL-13Rα2

| | IL-13Rα1, | IL-13Rα2, |
|---|---|---|
| Constructs | K_{D} (nM) | K_{D} (nM) |
| Fc-IL13 KIH | 470 | 0.15 |
| Fc-MDNA132.89 KIH | No binding | 20 |
| Fc-MDNA132.90 KIH | No binding | 5.62 |
| Fc-MDNA132.15 KIH | No binding | 0.3 |
| Fc-MDNA132.100 KIH | No binding | 2.12 |
| Fc-MDNA132.7 KIH | No binding | 2.53 |
| Fc-MDNA132.91 KIH | No binding | 8.03 |
| Fc-MDNA132 (historical data) | No binding | 2.5 |

### Conclusion

Fc-MDNA132.15 KIH exhibited enhanced binding affinity to IL-13Rα2 compared to the original parental Fc-MDNA132 and did not bind IL-13Rα1.

### Study 3: Generation of Stable Mouse Syngeneic Cell Lines Expressing IL-13Rα2

Available syngeneic tumor models/cell lines do not express IL-13Rα2, necessitating the need to engineer cell lines that stably overexpress this decoy receptor to facilitate efficacy studies in immune competent mice.

### Methodology

Synthesized IL-13Rα2 sequence was cloned into a T2A-PuroR lentiviral vector to manufacture lentiviral particles carrying the IL-13Rα2 transgene for stable expression in target cells lines EMT6 (breast cancer) and B16F10 (melanoma). Transduced cells were subjected to puromycin selection and pooled stabled clones were assessed by flow cytometry with an anti-IL-13Rα2 antibody to confirm surface expression of the decoy receptor.

### Results

EMT6 breast cancer cells were transduced with IL-13Rα2-T2A-PuroR lentivirus at multiplicity of infection (MOI) of 5, 10 and 25. Surface expression of IL-13Rα2 was assessed by flow cytometry and frequency of cells expressing the decoy receptor did not correlate with MOI for reasons that are unclear (data not shown). Nonetheless, EMT6 cells stably expressing IL-13Rα2 was successfully established.

The B16F10 cells transduced with IL-13Rα2-T2A-PuroR lentivirus did not grow well under puromycin selection. As a result, effort to establish IL-13Rα2 expressing B16F10 cells was not successful.

### Conclusion

EMT6 cells stably expressing IL-13Rα2 was successfully generated.

### Study 4: Ex Vivo Analysis of EMT6/IL-13Rα2 Tumors for Expression of IL-13Rα2

The objective was to confirm surface expression of IL-13Rα2 in established EMT6/IL-13Rα2 tumors in BALB/c mice.

### Methodology

Two naïve BALB/c mice were implanted with 5×10⁶ EMT6 cells in the left flank and 5×10⁶ EMT6/IL-13Rα2 cells in the right flank. Tumors were allowed to grow until they reached an average size of 160 mm³. The tumors were surgically removed and processed to single cell expansions for analysis of IL-13Rα2 expression by flow cytometry.

### Results

Flow cytometry data presented as scatter plots and histograms were obtained (data not shown). EMT6/IL-13Rα2 tumors and not EMT6 wild-type tumors showed surface expression of the IL-13 decoy receptor.

### Conclusion

EMT6/IL-13Rα2 tumors grown in Balb/c mice expressed the IL-13 decoy receptor IL-13Rα2.

### Study 5: In Vitro Receptor Internalization Assay

The objective was to investigate the internalization of MDNA132/IL-13Rα2 complex from cell surface. Data from this study inform the dynamic of receptor binding and subsequent internalization that may impact in vivo applications.

### Methodology

Cells were cultured in 6 well plates to reach 80-90% confluency. Cells were incubated with 500 nM concentration of Fc fused IL-13 variants for 15 min. Plates were washed three times with 6 mL of culture media/PBS and cells continued incubating in media for different lengths of time. At scheduled time-points (1-hr, 4-hr and 24-hr), stained cells were fixed in 2% paraformaldehyde and kept on ice until analysis. Fixed cells were analyzed by flow cytometry.

Cell lines used for the experiment: Genetically modified EMT6 cells expressing IL-13Rα2, U87, human glioma cells naturally expressing IL-13Rα2, Wild type EMT-6 cells not expressing IL-13Rα2.

TABLE 14
Binding K_{D} (nM) of anti-PD1-.15 to human
IL-13Rα1 and IL-13Rα2

| | IL-13Rα1, | IL-13Rα2, |
|---|---|---|
| Constructs | K_{D} (nM) | K_{D} (nM) |
| Anti-mPD1-MDNA132.15 (1:1 KIH) | No binding | 0.6 |

Anti-mPD1-MDNA132.15 was tested for binding to mouse PD1 and showed binding affinity (K_{D}) of 87.7 nM.

Fc^{act}-MDNA132.15 (1:1 KIH) was tested for binding affinity to IL-13 and Fc receptors. Active Fc-MDNA132.15 exhibited selective binding human IL-13Rα2 (K_{D} of 0.3 nM) and no binding to IL-13Rα1.

Active Fc-MDNA132.15 binds to both CD32b/c (K_{D} of 2850 nM) and CD16a (K_{D} of 538 nM) with affinity within the range reported in literature.

### Conclusions

MDNA132.15 binds selectively to IL-13 decoy receptor with no binding to IL-13Rα1, as anticipated. Data showed selective binding of Fc^{act}-MDNA132.15 to human IL-13 decoy receptor and to human Fc-receptors for effector function.

### PD1 Reporter Assay to Test PD1/PDL1 Blockade by Anti-PD1-MDNA132.15

In the PD-1/PD-L1 Blockade Bioassay, PD-L1 aAPC/CHO-K1 cells are used to engage PD-1 effector cells through the T-cell receptor (TCR) or the PD-1 receptor. When PD-L1 engages PD-1, TCR signaling, and the downstream luciferase reporter driven by an NFAT response element is inhibited. PD-1 blocking antibodies prevent the interaction of PD-1 and PD-L1, allowing TCR signaling and subsequent NFAT-reporter luminescence.

The objective was to measure the potency of anti-mPD1-MDNA132.15 in the mouse in vitro PD1 reporter assay.

### Methodology

Mouse PD-L1 aAPC/CHO-K1 cells were plated into 96 well plates 16 hours in a volume of 100 μL and incubated for 16 hours. Test articles were diluted as 2× solutions. Media was removed from the pre-plated reporter cells and 40 μL of the 2× test articles and 40 μL of the Mouse PD-1 T&U Effector Cells were added to each well. Plates were incubated for 6 hours, and luciferase substrate was added and incubated for additional 15 minutes prior to quantification on an iD5 plate reader.

### Results

Anti-mPD1 and anti-mPD1-MDNA132.15 were assessed in the mouse PD-1/PD-L1 Blockade Bioassay. EC₅₀ values were determined for each construct by plotting RLU versus the concentration and applying a 4-parameter logistic curve. Comparison of the EC₅₀ values indicated that anti-PD1 and anti-PD1-MDNA132.15 had similar potency of 5.9 nM and 6 nM respectively.

### Conclusion

Anti-mPD1-MDNA132.15 inhibited PD1/PDL-1 blockade with similar potency as parental anti-mPD1.

### Overall Conclusions

To summarize, IL-13 superkine, MDNA132 shows:
Selective affinity to IL-13 decoy receptor (IL-13Rα2) while exhibiting no binding to functional receptor, IL-13Rα1
The selectivity was maintained for murine and cynomolgus IL-13Rα2
Murine syngeneic breast tumor cell line, EMT6 was successfully transduced with IL-13Rα2 which was confirmed ex-vivo by flow cytometry..

### Study 8: In Vivo Imaging for Accumulation of Fc-MDNA132.15 in IL-13Rα2 Expressing Tumors in Mice

The objective of the study was to evaluate the distribution of Fc-MDNA132.15 in mice to examine selective accumulation in IL-13Rα2 expressing vs IL-13Rα2 non-expressing tumors. Hence, genetically modified cell line expressing IL-13Rα2, EMT6/IL13Rα2 and wild type EMT6 (not expressing the IL-13Rα2, as control) were used in the study. In addition, human cell line expressing IL-13Rα2, U87 glioblastoma and lung epithelial cell line A549 (not expressing the IL-13Rα2, as control) were used in study in athymic mice.

### Methodology

### Labelling of Fc-MDNA132.15

A VivoTag800 IN VIVO NIR fluorochrome (Perkin Elmer, cat. #NEV11107) was used to label Fc-MDNA132.15 according to the manufacturer's protocol. Fc-MDNA132.15 was first buffer exchanged into 1× PBS using Zeba7K MWCO columns (Thermo Fisher, cat. #89882) to provide a compatible buffer for successful labelling. Labeling was performed as follows:
1 mL of a 1 mg/mL solution of conjugation in conjugation buffer was prepared.
1 mg of VivoTag800 was reconstituted with 100 μL DMSO.
5-10 μL of VivoTag800 was added to protein solution and mixed well.
Reaction was incubated at room temperature for 1 hour.
Unreacted fluorophore was removed by size exclusion chromatography.
   Binding analysis by Biolayer Interferometry

To establish that labeling of Fc-MDNA132.15 did not alter its receptor binding profile, labelled and unlabeled Fc-MDNA132.15 was tested for binding to mouse IL13Rα2 by Biolayer Interferometry (BLI)/Octet.

The general BLI step conditions used were as follows:
Biosensors check in PBS Kinetics buffer (0.5 minutes),
ligand protein immobilization (10 minutes),
blocking with Superblock (2 minutes),
baseline in PBS Kinetics buffer (2 minutes),
analyte titration.

Ni-NTA biosensors (Forte Bio Part #18-5101) were used for each experiment in a 96-well plate (Greiner Part #655209) as required by the Octet RED96 Biolayer Inferometer (BLI) instrument manufacturer (Forte Bio). Polyhistidine-tagged Human IL-13Ral ligand (Sino Biological cat. #10943-H08H) or polyhistidine-tagged Human IL-13RA2 ligand (Sino Bilogical cat #10350-H08H) was immobilized to the Ni-NTA biosensors at 200 nM and then dipped into a titration of Human Fc-IL13 (AcroBiosystems cat. #IL3-H5256) and VivoTag800 IN VIVO NIR fluorochrome labelled and unlabeled Fc-MDNA132 analytes in PBS buffer (11.9 mM phosphate pH 7.4, 137 mM sodium chloride, 2.7 mM potassium chloride) containing Kinetics reagent (Forte Biocat. #18-1105).

### In Vivo Imaging

Since no murine cancer cell line is known to express IL13Rα2, EMT6 cells were genetically modified to express IL-13Rα2 and were used in this study (EMT6/IL-13Rα2) and EMT-6 wild type were used as control. Accordingly, Balb/c mice were used for implantation and tumor generation.

Furthermore, few human cell lines are known to express the IL-13 decoy receptor including glioblastoma cell line, U87 and hence, was used to determine accumulation of Fc-MDNA132.15 in this study. A549, human lung epithelial cell line, not known to express IL-13Rα2 was used as a control. Accordingly, athymic mice were used for implantation and tumor generation.

### Animal Information

Strain: BALB/c (strain code 028)
Vendor: Charles River Laboratories
Species: Mouse
Sex: Female
Age/Weight: 5-6 weeks
Number: 8 (5+3 extra) Animals will be purpose-bred, specific pathogen-free, and experimentally naïve at the start of the study. Age/Weight refers to the Age/Weight at the time of delivery from the vendor.

Strain: Athymic Nude (strain code 490)
Vendor: Charles River Laboratories
Species: Mouse
Sex: Female
Age/Weight: 5-6 weeks
Number: 7 (5+2 extra) Animals will be purpose-bred, specific pathogen-free, and experimentally naïve at the start of the study. Age/Weight refers to the Age/Weight at the time of delivery from the vendor.

Animals were allowed to acclimate to the housing environment (the animal holding room) for at least 3 days prior to the initiation of the study. During the acclimation period, the general health of the animals was monitored. Animals that appeared normal and did not exhibit signs of poor health were enrolled in the study and randomized prior to dosing. Animals that had: 1) significantly low body weight, 2) severe dehydration, or 3) wounds from fighting were excluded from the study.

The animals were monitored for tumor growth starting 3-5 days post inoculation. When average tumor volume of the tumor on the left flank (EMT6.13Rα2) reached between 300-500 mm³, animals were stratified into groups prior to test article administration to obtain similar average tumor volume among groups.

The study design is shown in Table 15.

TABLE 15
Study Design for in vivo imaging with Fc-MDNA132.15

| | | | | Dosing route, | |
|---|---|---|---|---|---|
| | No. of | | | Frequency | |
| Group | animals | Cell line | Test Agent | and duration | Study tasks |
| 1 | 3 | EMT6 | Fc- | IV, Single Dose | Tumor |
| | | 5.0 × 10⁶ cells in 0.1 mL | MDNA132.15 (2 | on Day 0 at T = 0 | measurements |
| | | volume | mg/kg) | | (2x times/week) |
| | | per animals (50% | Concentration: | | Body weights (2x |
| | | Matrigel) | 0.4 mg/mL | | times/week) |
| | | Subcutaneous in animals' | Volume: | | Clinical |
| | | left | 5 mL/kg | | Observations (2x |
| | | flank region | | | times/week) |
| 2 | | EMT6/IL13Ra2 | Background | | IVIS Imaging (in |
| | | 2.0 × 10⁶ cells in 0.1 mL | control | | vivo at pre-dose, |
| | | volume | | | 4 h, 24 h, 72 h, 96 h, |
| | | per animals (50% | | | and 120 h post |
| | | Matrigel) | | | dose and ex vivo |
| | | Subcutaneous in animals' | | | at termination) |
| | | right | | | |
| | | flank region | | | |
| 3 | 3 | A549 | Fc- | | |
| | | 5.0 × 10⁶ cells in 0.1 mL | MDNA132.15 (2 | | |
| | | volume | mg/kg) | | |
| | | per animals (50% | Concentration: | | |
| | | Matrigel) | 0.4 mg/mL | | |
| | | Subcutaneous in animals' | Volume: | | |
| | | left | 5 mL/kg | | |
| | | flank region | | | |
| 4 | | U87 mg | Background | | |
| | | 5.0 × 10⁶ cells in 0.1 mL | control | | |
| | | volume | | | |
| | | per animals (No Matrigel) | | | |
| | | Subcutaneous in animals' | | | |
| | | right | | | |
| | | flank region | | | |

Epifluorescence IVIS imaging using excitation=785 nm and emission=810 nm in dorsal position at pre-dose for baseline image was performed at 4 h, 24h, 72h, 96h, and 120 h post-injection of labelled MDNA109FEAA-Fc-MDNA 132.15. Following the last imaging timepoint (120 hr post-injection), tumors were collected for ex-vivo imaging.

### Results

Labelling ofMDNAI19FEAA-Fc-MDNA132.15

Fluorescence labeling was performed on both Fc-MDNA132.15 and MDNA109FEAA-Fc-MDNA132.15.

The data that was referred to for confirming the labeling experiment is shown in Table 16. The absorbance at 790 nm and the protein: vivotag800 ratio of 2.3 clearly indicate that the construct was successfully labelled.

TABLE 16
Protein labeling and absorbance data for MDNA132 constructs and purification

| | | | A280 after | Protein | | | | | |
|---|---|---|---|---|---|---|---|---|---|
| | | | labelling and | Concen- | | A790 | | | |
| | | | purification | tration | | after | | | Final |
| | | | (Adjusted | (Not Including | Protein | labelling | VivoTag800 | | Labeled |
| | Protein | | for A790 | VivoTag800 | Concen- | and | Concen- | Protein:VivoTag800 | Protein |
| Protein | MW | OD0.1% | contribution) | Mass) | tration | purification | tration | Ratio | Volume |
| Fc-MDNA132.15 | 63.9 kDa | 1.27 | 1.42 | 1.12 mg/mL | 17.5 μM | 5.9 | 29.7 μM | 1.7 | 3.3 mL |

The labelled constructs were tested for binding to mouse IL-13 and IL-2 receptors, IL-13Rα2 and CD122 by Octet before testing the same in in vivo localization study.

### Binding Analysis by BLI/Octet

Binding affinity by BLI/Octet was performed to confirm that the receptor binding profile of Fc-MDNA132.15 was not modified by labelling with fluorophore. Both unlabeled and labelled Fc-MDNA132.15 showed similar binding properties to IL-13Rα2 and CD122.

For mouse IL-13Rα2, the calculated K_{D} was 0.48 nM for Fc-MDNA132.15, however, the K_{D} for labeled Fc-MDNA132.15 could not be calculated because the off rate was below detection limit. Nevertheless, the sensorgrams clearly indicate binding affinity of the construct to mouse IL-13Rα2.

### In Vivo Imaging

EMT6 wild type and modified EMT6/IL-13Rα2 cells were engrafted in either flank of Balb/c mice and allowed to grow into established tumors. Body weight measurements and clinical observations were noted twice weekly. A549 and U87 cells were engrafted in either flank of athymic nude mice and allowed to grow into established tumors. Body weight measurements and clinical observations were noted twice weekly.

Mice with established tumors were IV injected with labelled Fc-MDNA132.15 and IVIS images were acquired pre-dose, 4 h, 24h, 72h, 96h, and 120 h post-treatment.

Accumulation of labelled Fc-MDNA132.15 was observed in EMT6/IL13Rα2 tumors (right flank) as compared to in EMT6 wild type tumors (left flank). At earlier time-point (i.e., up to 24 hours), Fc-MDNA132.15 could be observed distributed throughout the body of the mice. As Fc-MDNA132.15 is cleared from system distribution at latter time-points (72 to 144 hours), accumulation in IL-13Rα2 expressing tumors was evident in most mice. The ability of Fc-MDNA132.15 to localize to EMT6/IL-13Rα2 tumors validated the previous observations of accumulation of labeled Fc-MDNA132 in U87 tumors. Following in vivo imaging at the 144-hour time-point, mice were euthanized, and their intact tumors and were extracted for ex-vivo imaging.

Similarly, accumulation of labelled Fc-MDNA132.15 was observed in U87 tumors (right flank) as compared to in A549 tumors (left flank). However, in contrast to above observations, Fc-MDNA132.15 could be observed distributed throughout the body of the mice up to 168 h. Accumulation was though observed in IL-13Rα2 expressing U87 tumors in most mice compared to A549 tumors. The ability of Fc-MDNA132.15 to localize to U87 tumors also corroborated with previous observations. Following in vivo imaging at the 168-hour time-point, mice were euthanized, and their intact tumors and were extracted for ex-vivo imaging wherein the evidence of Fc-MDNA132.15 in U87 was more pronounced.

### Conclusions

BLI/Octet studies showed that labelled Fc-MDNA132.15 retained the expected receptor binding profile as unlabeled Fc-MDNA132.15 (i.e., high affinity to mouse IL-13Rα2).

In vivo and ex vivo imaging showed prolonged and durable accumulation of Fc-MDNA132.15 in IL-13Rα2 expressing EMT6 and U87 tumors.

### Example 2: Evaluation of Tumor Growth Inhibition in Murine Prostate Cancer Model TRAMP-C1

### Method

40 Male C57BI/6 mice were implanted with 2×10⁶ cells and randomized 3 days after implantation. Post implantation, animals were dosed per the study design in Table.

| | Animals/ | | | | Route of | |
|---|---|---|---|---|---|---|
| Group | Group | Compound | Dose | | administration | Frequency of dosing |
| 1 | 10 | Vehicle | — | | IP | Twice weekly x3 |
| 2 | 10 | Fc-MDNA413 | 30 | mg/kg | IP | Twice weekly x3 |
| | | | | | | (Starting first week) |
| 3 | 10 | MDNA19 | 5 | mg/kg | IP | Once weekly x2 |
| | | | | | | (Starting second week) |
| 4 | 10 | Fc-MDNA413 | 30 | mg/kg | IP | Twice weekly x3 |
| | | | | | | (Starting first week) |
| | | MDNA19 | 5 | mg/kg | IP | Once weekly x2 |
| | | | | | | (Starting second week) |

### Results:

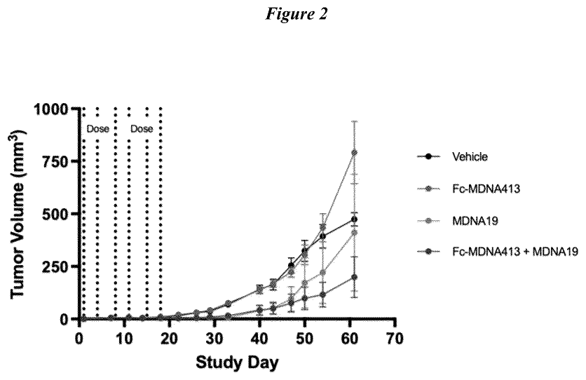

Fc-MDNA413 demonstrates tumor growth similar to vehicle control and MDNA19 exhibits moderate tumor growth inhibition in the TRAMP-C1 prostate tumor model. However, the combination of Fc-MDNA413 and MDNA19 shows superior tumor growth inhibition compared to either of the agents alone. See FIG. 2.

The data is consistent with the observations in the B16F10 melanoma model. Like B16F10, TRAMP-C1 prostate model is regarded as immunologically ‘cold’ tumor. Prostate tumors are characterized by low infiltration of T-cells, absence of type I interferon (IFN) and abundance of immunosuppressive cells (myeloid cells and tumor associated macrophages). The data strengthens the therapeutic potential of combination treatment with Fc-MDNA413 and MDNA19.

### Example 3: Cytotoxicity Assay

### Method:

Cells were plated at a density of 10,000 per well in 96 well plates in culture media a day before assay initiation (Day 0). On Day 1, cells were treated with the constructs at various concentrations. Constructs were each diluted in the respective cell culture media. Culture media on plates was replaced with 200 μL of media containing each concentration of the constructs.

Fc-MDNA132.15 (KIH) was used as a negative control while Blasticidin at 30 μg/mL was used as positive control in the assay. Wells containing untreated cells were included as experimental controls. Plates were incubated for either 48 or 72 hours. Post incubation time, plates were processed by adding 20 μL of Cell Titer-Blue Reagent (Promega G8080) to each well and incubating the plates for 3 hours at 37° C./5% C02. After completion of incubation period, plates were scanned for fluorescence at 555 nm excitation and 595 nm emission.

### Result:

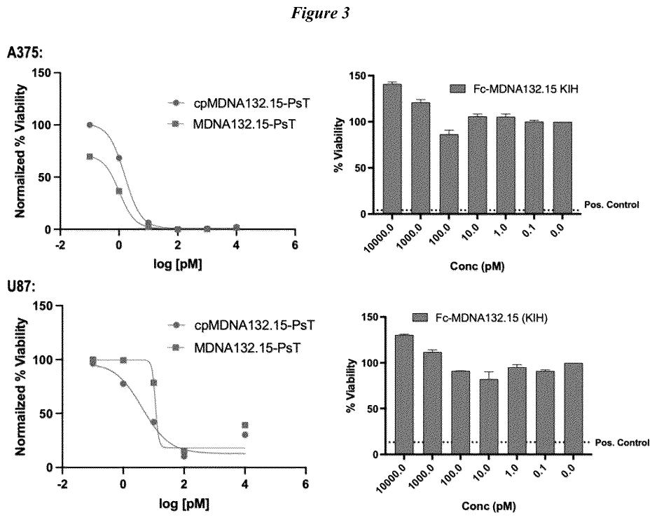

The human melanoma cell line, A375 and human glioblastoma cell line, U87 both express the IL-13 decoy receptor. The results for 48 h incubation in A375 and U87 are presented in FIG. 3. As observed, both MDNA132.15-PE and cpMDNA132.15-PE induced significant cytotoxicity in both human cell lines with A375 showing a better response than U87. Moreover, as expected, Fc-MDNA132.15 (KIH) did not show any cytotoxicity in both the cell lines (FIG. 3, right panel, grey bars).

Cytotoxicity of the MDNA132.15-exotoxin fusions were also tested in murine breast cancer cell line EMT6 that was engineered to express IL13Rα2 (EMT6-IL13Rα2). In addition, to evaluate the specificity of cytotoxicity to IL13Rα2 expressing cell lines, EMT6 wild type cell line that does not express IL13Rα2 was subjected to MDNA132.15-exotoxin fusion construct treatment. The results for 48 h incubation are shown in FIG. 4.

As observed, both MDNA132.15-PE and cpMDNA132.15-PE (also referred to as MDNA132.15-PsT and cpMDNA132.15-PsT, respectively) induced significant cytotoxicity in EMT6-IL13Rα2 cells specifically with no cell death observed in EMT6 wild type cells at most concentrations tested

The estimated IC₅₀s (inhibitory concentrations where 50% viability is lost or observed) are summarized in Table 17.

TABLE 17
IC₅₀ (pM)of the tested constructs in the cytotoxicity assay
for various cell lines with 48 h incubation period.

| Test Article | Cell Line | IC₅₀ (pM) |
|---|---|---|
| cpMDNA132.15-PE | A375 | 1.5 |
| | U87 | 4.2 |
| | EMT6-IL13Rα2 | 1.0 |
| MDNA132.15-PE | A375 | 1.02 |
| | U87 | 11.4 |
| | EMT6-IL13Rα2 | 2.1 |

To assess the effect of incubation time on cytotoxicity, the assay in all the cell lines was also performed for 72 h.

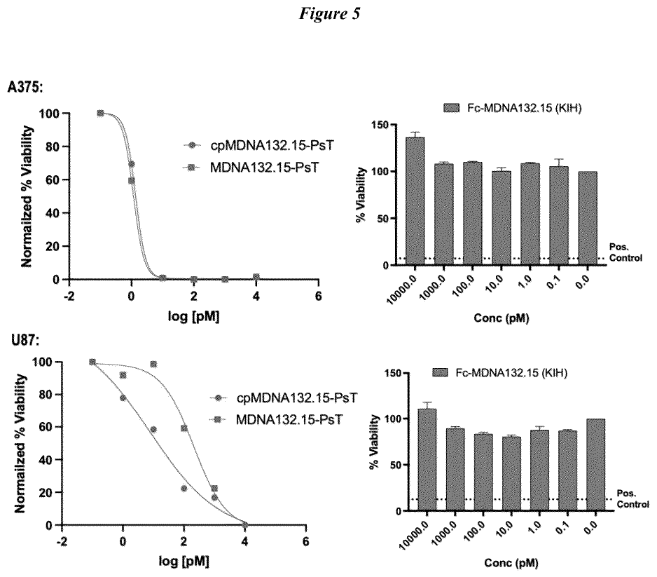

The results for 72 h incubation in A375 and U87 are presented in FIG. 5. MDNA132.15-PE and cpMDNA132.15-PE induced significant cytotoxicity in both cell lines. The dose dependent response observed with A375 was superior to that observed with U87. Consistent with 48 h incubation data, Fc-MDNA132.15 (KIH) did not show any cytotoxicity in both the cell lines (FIG. 5, right panel, grey bars) on 72 h of incubation.

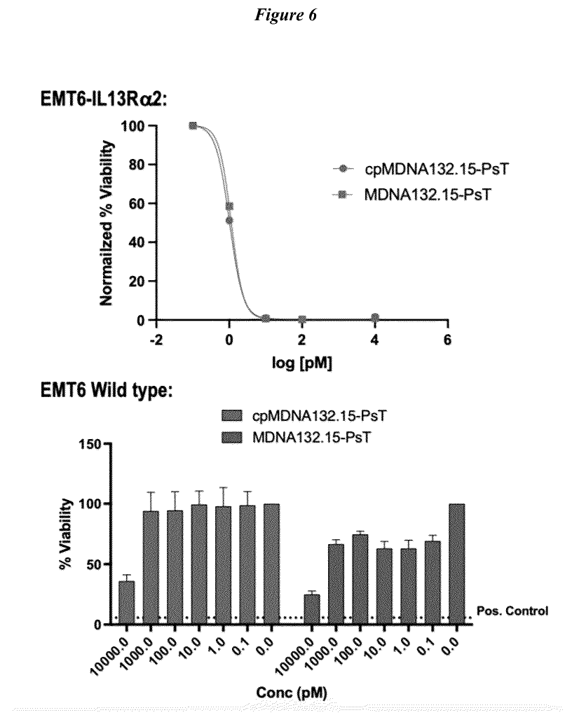

Similarly, the data for 72-h incubation set up in EMT6 wild type and genetically engineered EMT6-IL13Rα2 is shown in FIG. 6.

Both MDNA132.15-PE and cpMDNA132.15-PE induced substantial cytotoxicity in EMT6-IL13Rα2 cells only and no significant cell death was observed in EMT6 wild type cells at most concentrations tested.

The estimated IC₅₀s (inhibitory concentrations where 50% viability is lost or observed) are summarized in Table 18.

TABLE 18
IC₅₀ (pM) of the tested constructs in the cytotoxicity assay
for various cell lines with 72 h incubation period.

| Test Article | Cell Line | IC₅₀ (pM) |
|---|---|---|
| cpMDNA132.15-PE | A375 | 1.3 |
| | U87 | 7.6 |
| | EMT6-IL13Rα2 | 1.0 |
| MDNA132.15-PE | A375 | 1.1 |
| | U87 | 207.8 |
| | EMT6-IL13Rα2 | 1.13 |

Treatment of IL13Rα2 expressing cell lines with both MDNA132.15-PE and cpMDNA132.15-PE for either 48 h or 72 h gave comparable IC₅₀ values validating the cytotoxic potential of the MDNA132.15-exotoxin fusion constructs.

### Example 4: In Vivo Tumor Growth Inhibition Assay

Tumor bearing mice were treated with either MDNA132.15 (SEQ ID NO: 106 also referred to as MDNA213) fused to Pseudomonas aeruginosa exotoxin (PE) 15 100 μg/kg IT and/or MDNA19 (i.e. MDNA109FEAA-Fc, an IL-2 super-agonist, SEQ ID NO:31) 5 mg/kg once weekly X 3 IP, days 3, 10 and 17. Tumor volume was assessed at 0, 2.5, 5.0, 7.5, 10, 12.5, and 15 days.

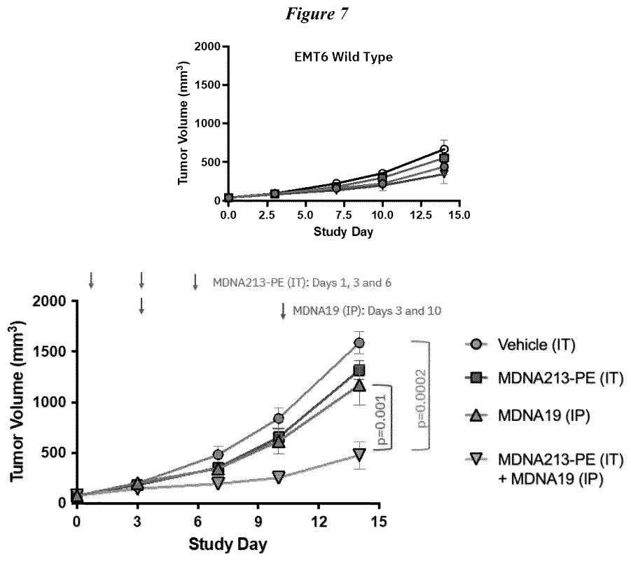

As shown in FIG. 7, MDNA213-PE exhibited tumor growth inhibition in EMT6-IL13Rα2 tumors specifically (bottom) with an insignificant response in EMT-6 wild type tumors (top). The MDNA213-PE response was observed to synergize in combination with IL-2 super-agonist, MDNA19 to significantly enhance the therapeutic efficacy.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A method of treating a cancer in a patient in need thereof comprising administering to the patient:
a) an IL-4 receptor targeted cargo protein or an IL-13 receptor targeted cargo protein; and
b) an immunomodulatory agent,
wherein the IL-4 receptor targeted cargo protein comprises an IL-4, IL-13, or IL-4 mutein, or an IL-13 mutein linked to a cargo moiety,
wherein the IL-13 receptor targeted cargo protein comprises an IL-4, IL-13, an IL-4 mutein, or an IL-13 mutein linked to a cargo moiety,
wherein the cargo moiety is capable of inhibiting cancer cell growth or killing cancer cells.

2. The method of claim 1, wherein the IL-4 or IL-4 mutein is selected from the IL-4 muteins in Table 6.

3. The method of claim 1, wherein the IL-4 receptor targeted cargo protein has the amino acid sequence of SEQ ID NO:1057.

4. The method of claim 1, wherein the IL-13 or IL-13 mutein is selected from the IL-13 muteins disclosed in Tables 5, 7, and 8.

5. The method of claim 4, wherein the IL-13 mutein has the amino acid sequence of SEQ ID NO:106.

6. The method of claim 4, wherein the IL-13 mutein has the amino acid sequence of SEQ ID NO:159.

7. The method of claim 4, wherein the IL-13 mutein has the amino acid sequence of SEQ ID NO:105.

8. The method of claim 4, wherein the IL-13 mutein has the amino acid sequence of SEQ ID NO:110.

9. The method of claim 4, wherein the IL-13 mutein has the amino acid sequence of SEQ ID NO:118.

10. The method of claim 1, wherein the immunomodulatory agent is an IL-2 mutein or IL-2 mutein fusion protein.

11. The method of claim 1, wherein the immunomodulatory agent is selected from an immune checkpoint inhibitor and a fusion protein disclosed in Tables 4 and 8.

12. The method of claim 10, wherein the IL-2 mutein or IL-2 mutein fusion protein is selected from IL-2 mutein or IL-2 mutein fusion protein in Tables 2-4.

13. The method of claim 12, wherein the IL-2 mutein fusion protein has the amino acid of SEQ ID NO:31.

14. The method of any one of claims 1 to 13 wherein the cargo moiety is selected from Pseudomonas exotoxin, Aerolysin, Proaerolysin, Bouganin, a Cholera toxin, and Ribonuclease A.

15. The method of claim 14, wherein the cargo moiety is Pseudomonas exotoxin.

16. The method of any one of claims 1 to 15, wherein the cancer is associated with IL-4R overexpression or IL-13Rα2 overexpression.

17. The method of any one of claims 1 to 16, wherein the cancer is refractory to immune-checkpoint inhibition.

18. The method of any one of claims 1 to 16, wherein the cancer is selected from the group consisting of sarcoma, carcinoma, head and neck cancer, glioblastoma, bladder cancer, oral cancer, mesothelioma, pancreatic cancer, liver cancer, colorectal cancer, pulmonary cancer, cutaneous, lymphoid, gastrointestinal cancer, prostate cancer, ovarian cancer, breast cancer, basal-like breast tumor, endometrial cancer, multiple myeloma, melanoma, lymphoma, lung cancer (including small cell lung cancer), kidney cancer, gastric cancer, brain cancer, and CNS tumors.

19. The method of any one of claims 1 to 18, wherein the IL-4 receptor targeted cargo protein or an IL-13 receptors targeted cargo protein is administered before the immunomodulatory agent.

20. The method of any one of claims 1 to 18, wherein the IL-4 receptor targeted cargo protein or IL-13 receptor targeted cargo protein is administered after the immunomodulatory agent.

21. The method of any one of claims 1 to 18, wherein the IL-4 receptor targeted cargo protein or IL-13 receptor targeted cargo protein is administered contemporaneously with the immunomodulatory agent.

22. A composition comprising:
a) an IL-4 receptor targeted cargo protein or an IL-13 receptor targeted cargo protein; and
b) an immunomodulatory agent,
wherein the IL-4 receptor targeted cargo protein comprises an IL-4, an IL-13, an IL-4 mutein, or an IL-13 mutein linked to a cargo moiety,
wherein the IL-13 receptor targeted cargo protein comprises an IL-4, an IL-13, an IL-4 mutein, or an IL-13 mutein linked to a cargo moiety, and
wherein the cargo moiety is capable of inhibiting cancer cell growth or killing cancer cells.

23. The composition of claim 22, wherein the IL-4 or IL-4 mutein is selected from the IL-4 muteins in Table 6.

24. The composition of claim 22, wherein the IL-4 receptor targeted cargo moiety has the amino acid sequence of SEQ ID NO:1057.

25. The composition of claim 22, wherein the IL-13 or IL-13 mutein is selected from the IL-13 muteins disclosed in Tables 5, 7 and 8.

26. The composition of claim 25, wherein the IL-13 mutein has the amino acid sequence of SEQ ID NO:106.

27. The composition of claim 25, wherein the IL-13 mutein has the amino acid sequence of SEQ ID NO:159.

28. The composition of claim 25, wherein the IL-13 mutein has the amino acid sequence of SEQ ID NO:105.

29. The composition of claim 25, wherein the IL-13 mutein has the amino acid sequence of SEQ ID NO:110.

30. The composition of claim 25, wherein the IL-13 mutein has the amino acid sequence of SEQ ID NO: 118.

31. The composition of claim 22, wherein the immunomodulatory agent is an IL-2 mutein or IL-2 mutein fusion protein.

32. The composition of claim 22, wherein the immunomodulatory agent is selected from an immune checkpoint inhibitor and a fusion protein disclosed in Tables 4 and 8.

33. The composition of claim 31, wherein the IL-2 mutein or IL-2 mutein fusion protein is selected from IL-2 mutein or IL-2 mutein fusion protein in Tables 2-4.

34. The composition of claim 33, wherein the II-2 mutein fusion protein has the amino acid of SEQ ID NO:31.

35. The composition of claim 22-34, wherein the cargo moiety is selected from Pseudomonas exotoxin, Aerolysin, Proaerolysin, Bouganin, a Cholera toxin, and Ribonuclease A.

36. The composition of claim 35, wherein the cargo moiety is Pseudomonas exotoxin.


SEQUENCE LISTING (1068 sequences)

SEQ ID NO: 1 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 2 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 3 - protein, 133 aa, synthetic construct
APTSSSTKKTQLQLEHLLLDLQMILNGINNYKNPKLTRMLTFNFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 4 - protein, 133 aa, synthetic construct
APTSSSTKKTQLQLEHLLLDLQMILNGINNYKNPKLTRMLTAKFAMPKKATELKHLQCLEEELKPLEEVLNLAQSKNFHFDPRDVVSNINVFVLELKGSETTFMCEYADETATIVEFLNRWITFCQSIISTLT

SEQ ID NO: 5 - protein, 133 aa, synthetic construct
APTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCQSIISTLT

SEQ ID NO: 6 - protein, 133 aa, synthetic construct
APTSSSTKKTQLQLEHLLLDLQMILNGINNYKNPKLTRMLTAKFAMPKKATELKHLQCLEEALKPLEEVLNLAQSKNFHFDPRDVVSNINVFVLELKGSETTFMCEYADETATIVEFLNRWITFCQSIISTLT

SEQ ID NO: 7 - protein, 133 aa, synthetic construct
APTSSSTKKTQLQLEHLLLDLQMILNGINNYKNPKLTRMLTFKFYMPKKATELKHLQCLEEELKPLEEVLNLARSKNFHLRPRDLISNINVIVLELKGSETTFMCEYADETATIVEFLNRWITFCQSIISTLT

SEQ ID NO: 8 - protein, 133 aa, synthetic construct
APTSSSTKKTQLQLEHLLLDLQMILNGINNYKNPKLTRMLTFKFYMPKKATELKHLQCLEEELKPLEEVLNLARSKNFHLRPRDVISNINVIVLELKGSETTFMCEYADETATIVEFLNRWITFCQSIISTLT

SEQ ID NO: 9 - protein, 133 aa, synthetic construct
APTSSSTKKTQLQLEHLLLDLQMILNGINNYKNPKLTRMLTFKFYMPKKATELKHLQCLEEELKPLEEVLNLARSKNFHLIPRDVISNINVIVLELKGSETTFMCEYADETATIVEFLNRWITFCQSIISTLT

SEQ ID NO: 10 - protein, 133 aa, synthetic construct
APTSSSTKKTQLQLEHLLLDLQMILNGINNYKNPKLTRMLTFKFYMPKKATELKHLQCLEEELKPLEEVLNLAHSKNFHLTPRDVVSNINVFILELKGSETTFMCEYADETATIVEFLNRWITFCQSIISTLT

SEQ ID NO: 11 - protein, 133 aa, synthetic construct
APTSSSTKKTQLQLEHLLLDLQMILNGINNYKNPKLTRMLTFKFYMPKKATELKHLQCLEEELKPLEEVLNLANSKNFHFDPRDVVSNVNVFVLELKGSETTFMCEYADETATIVEFLNRWITFCQSIISTLT

SEQ ID NO: 12 - protein, 133 aa, synthetic construct
APTSSSTKKTQLQLEHLLLDLQMVLNGINNYKNPKLTRMLTFKFYMPKKATELKHLQCLEEELKPLEEVLNLASSKNFHFDPRDVVSNINVFVLELKGSETTFMCEYADETATIVEFLNRWITFCQSIISTLT

SEQ ID NO: 13 - protein, 133 aa, synthetic construct
APTSSSTKKTQLQLEHLLLDLQMILNGINNYKNPKLTRMLTFKFYMPKKATELKHLQCLEEELKHLEEVLNLANSKNFHVTPRDVVSNINVFVLELKGSETTFMCEYADETATIVEFLNRWITFCQSIISTLT

SEQ ID NO: 14 - protein, 133 aa, synthetic construct
APTSSSTKKTQLQLEHLLLDLQMILNGINNYKNPKLTRMLTFKFYMPKKATELKHLQCLEEELKPLEEVLNLAHSKNFHFDPRDVVSNINVFVLELKGSETTFMCEYADETATIVEFLNRWITFCQSIISTLT

SEQ ID NO: 15 - protein, 133 aa, synthetic construct
APTSSSTKKTQLQLEHLLLDLQMILNGINNYKNPKLTRMLTFKFYMPKKATELKHLQCLEEELKPLEEVLNLASSKNFHFDPRDVVSNINVFVLELKGSETTFMCEYADETATIVEFLNRWITFCQSIISTLT

SEQ ID NO: 16 - protein, 133 aa, synthetic construct
APTSSSTKKTQLQLEHLLLDLQMILNGINNYKNPKLTRMLTFKFYMPKKATELKHLQCLEEELKPLEEVLNLANSKNFHFDPRDVVSNINVFVLELKGSETTFMCEYADETATIVEFLNRWITFCQSIISTLT

SEQ ID NO: 17 - protein, 133 aa, synthetic construct
APTSSSTKKTQLQLEHLLLDLQMILNGINNYKNPKLTRMLTFKFYMPKKATELKHLQCLEEELKPLEEVLNLASSKNFHLTPRDVISNINVFVLELKGSETTFMCEYADETATIVEFLNRWITFCQSIISTLT

SEQ ID NO: 18 - protein, 133 aa, synthetic construct; identical to SEQ ID NO: 11

SEQ ID NO: 19 - protein, 133 aa, synthetic construct
APTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFSQSIISTLT

SEQ ID NO: 20 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCTSIISTLT

SEQ ID NO: 21 - skipped

SEQ ID NO: 22 - protein, 375 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 375 aa, synthetic construct; identical to SEQ ID NO: 22

SEQ ID NO: 24 - protein, 375 aa, synthetic construct
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 375 aa, synthetic construct; identical to SEQ ID NO: 24

SEQ ID NO: 26 - protein, 375 aa, synthetic construct
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 374 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 29 - protein, 375 aa, synthetic construct
APTSSSTKKTQLQLEHLLLDLQMILNGINNYKNPKLTRMLTAKFAMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 375 aa, synthetic construct; identical to SEQ ID NO: 29

SEQ ID NO: 31 - protein, 375 aa, synthetic construct
APTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 375 aa, synthetic construct; identical to SEQ ID NO: 31

SEQ ID NO: 33 - protein, 375 aa, synthetic construct
APTSSSTKKTQLQLEHLLLDLQMILNGINNYKNPKLTRMLTAKFAMPKKATELKHLQCLEEALKPLEEVLNLAQSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 375 aa, synthetic construct
APTSSSTKKTQLQLEHLLLDLQMILNGINNYKNPKLTRMLTFKFYMPKKATELKHLQCLEEELKPLEEVLNLAHSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 375 aa, synthetic construct
APTSSSTKKTQLQLEHLLLDLQMILNGINNYKNPKLTRMLTFKFYMPKKATELKHLQCLEEELKPLEEVLNLASSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 375 aa, synthetic construct
APTSSSTKKTQLQLEHLLLDLQMILNGINNYKNPKLTRMLTFKFYMPKKATELKHLQCLEEELKPLEEVLNLANSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 375 aa, synthetic construct
APTSSSTKKTQLQLEHLLLDLQMILNGINNYKNPKLTRMLTFKFYMPKKATELKHLQCLEEELKPLEEVLNLANSKNFHFDPRDVVSNVNVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 733 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 39 - protein, 733 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPTSSSTKKTQLQLEHLLLDLQMILNGINNYKNPKLTRMLTAKFAMPKKATELKHLQCLEEELKPLEEVLNLAQSKNFHFDPRDVVSNINVFVLELKGSETTFMCEYADETATIVEFLNRWITFCQSIISTLT

SEQ ID NO: 40 - protein, 733 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCQSIISTLT

SEQ ID NO: 41 - protein, 733 aa, synthetic construct
APTSSSTKKTQLQLEHLLLDLQMILNGINNYKNPKLTRMLTFKFYMPKKATELKHLQCLEEELKPLEEVLNLAHSKNFHFDPRDVVSNINVFVLELKGSETTFMCEYADETATIVEFLNRWITFCQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 42 - protein, 733 aa, synthetic construct
APTSSSTKKTQLQLEHLLLDLQMILNGINNYKNPKLTRMLTAKFYMPKKATELKHLQCLEEALKPLEEVLNLAHSKNFHFDPRDVVSNINVFVLELKGSETTFMCEYADETATIVEFLNRWITFCQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 43 - protein, 733 aa, synthetic construct
APTSSSTKKTQLQLEHLLLDLQMILNGINNYKNPKLTRMLTFKFYMPKKATELKHLQCLEEELKPLEEVLNLASSKNFHFDPRDVVSNINVFVLELKGSETTFMCEYADETATIVEFLNRWITFCQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 44 - protein, 733 aa, synthetic construct
APTSSSTKKTQLQLEHLLLDLQMILNGINNYKNPKLTRMLTFKFYMPKKATELKHLQCLEEELKPLEEVLNLANSKNFHFDPRDVVSNINVFVLELKGSETTFMCEYADETATIVEFLNRWITFCQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 45 - protein, 733 aa, synthetic construct
APTSSSTKKTQLQLEHLLLDLQMILNGINNYKNPKLTRMLTFKFYMPKKATELKHLQCLEEELKPLEEVLNLANSKNFHFDPRDVVSNVNVFVLELKGSETTFMCEYADETATIVEFLNRWITFCQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 46 - protein, 733 aa, synthetic construct
APTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 47 - protein, 733 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPTSSSTKKTQLQLEHLLLDLQMILNGINNYKNPKLTRMLTFKFYMPKKATELKHLQCLEEELKPLEEVLNLAHSKNFHFDPRDVVSNINVFVLELKGSETTFMCEYADETATIVEFLNRWITFCQSIISTLT

SEQ ID NO: 48 - protein, 733 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPTSSSTKKTQLQLEHLLLDLQMILNGINNYKNPKLTRMLTAKFYMPKKATELKHLQCLEEALKPLEEVLNLAHSKNFHFDPRDVVSNINVFVLELKGSETTFMCEYADETATIVEFLNRWITFCQSIISTLT

SEQ ID NO: 49 - protein, 733 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPTSSSTKKTQLQLEHLLLDLQMILNGINNYKNPKLTRMLTFKFYMPKKATELKHLQCLEEELKPLEEVLNLASSKNFHFDPRDVVSNINVFVLELKGSETTFMCEYADETATIVEFLNRWITFCQSIISTLT

SEQ ID NO: 50 - protein, 733 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPTSSSTKKTQLQLEHLLLDLQMILNGINNYKNPKLTRMLTFKFYMPKKATELKHLQCLEEELKPLEEVLNLANSKNFHFDPRDVVSNINVFVLELKGSETTFMCEYADETATIVEFLNRWITFCQSIISTLT

SEQ ID NO: 51 - protein, 733 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPTSSSTKKTQLQLEHLLLDLQMILNGINNYKNPKLTRMLTFKFYMPKKATELKHLQCLEEELKPLEEVLNLANSKNFHFDPRDVVSNVNVFVLELKGSETTFMCEYADETATIVEFLNRWITFCQSIISTLT

SEQ ID NO: 52 - protein, 733 aa, synthetic construct
APTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFSQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 53 - protein, 733 aa, synthetic construct; identical to SEQ ID NO: 46

SEQ ID NO: 54 - protein, 375 aa, synthetic construct; identical to SEQ ID NO: 24

SEQ ID NO: 55 - protein, 374 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 56 - protein, 374 aa, synthetic construct; identical to SEQ ID NO: 28

SEQ ID NO: 57 - protein, 733 aa, synthetic construct
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 58 - protein, 374 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCQSIISTLT

SEQ ID NO: 59 - protein, 375 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCQSIISTLT

SEQ ID NO: 60 - protein, 375 aa, synthetic construct
APTSSSTKKTQLQLEHLLLDLQMILNGINNYKNPKLTWMLTFKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 375 aa, synthetic construct
APTSSSTKKTQLQLEHLLLDLQMILNGINNYKNPKLTRMLTAKFYMPKKATELKHLQCLEEALKPLEEVLNLANSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 375 aa, synthetic construct
APTSSSTKKTQLQLEHLLLDLQMILNGINNYKNPKLTRMLTAKFAMPKKATELKHLQCLEEELKPLEEVLNLANSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 375 aa, synthetic construct
APTSSSTKKTQLQLEHLLLDLQMILNGINNYKNPKLTWMLTAKFYMPKKATELKHLQCLEEALKPLEEVLNLANSKNFHFDPRDVVSNINVFVLELKGSETTFMCEYADETATIVEFLNRWITFCQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 375 aa, synthetic construct; identical to SEQ ID NO: 63

SEQ ID NO: 65 - protein, 374 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFAMPKKATELKHLQCLEEELKPLEEVLNLAQSKNFHFDPRDVVSNINVFVLELKGSETTFMCEYADETATIVEFLNRWITFCQSIISTLT

SEQ ID NO: 66 - protein, 733 aa, synthetic construct
APTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFAQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 67 - protein, 733 aa, synthetic construct
APTSSSTKKTQLQLEHLLLDLQMILNGINNYKNPKLTRMLTAKFYMPKKATELKHLQCLEEALKPLEEVLNLAQSKNFHLRPRDLISNINVIVLELKGSETTFMCEYADETATIVEFLNRWITFCQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 68-72 - skipped (5 entries)

SEQ ID NO: 73 - protein, 113 aa, synthetic construct
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 74 - protein, 113 aa, synthetic construct
PGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 75 - protein, 113 aa, synthetic construct
PGPVPPSTAIRELIEELINITQNQKAPLCNGSMVWSINLTAGMYCAALESLINVSGCSAIEKTQRMLSGFCPHKVSAGQFSSLHVRGSKIEVAQFVKDLLHHLRALFREGQFN

SEQ ID NO: 76 - protein, 113 aa, synthetic construct
PGPVPPSTALIELIEELINITQNQKAPLCNGSMVWSINLTAGIYCAALESLINVSGCSAIEKTQRMLSGFCPHKVSAGQFSSLHVKGSKIEVAQFVKDLLHHLRALMREGQFN

SEQ ID NO: 77 - protein, 113 aa, synthetic construct
PGPVPPSTAIRELIEELLNITQNQKAPLCNGSMVWSINLTAGMYCAALESLINVSGCSAIEKTQRMLSGFCPHKVSAGQFSSLHVMKSKIEVAQFVKDLLHHLRALFREGQFN

SEQ ID NO: 78 - protein, 113 aa, synthetic construct
PGPVPPSTAIRELIEELINITQNQKAPLCNGSMVWSINLTAGMYCAALESLINVSGCSAIEKTQRMLSGFCPHKVSAGQFSSLHVRSSRIEVAQFVKDLLHHLRTLFREGQFN

SEQ ID NO: 79 - protein, 113 aa, synthetic construct
PGPVPPSTALRELIEELINITQNEKAPLCNGSMVWSINLTAGIYCAALESLINVSGCSAIEKTQRMLSGFCPHKVSAGQFSSLHVTGSKIEVAQFVKDLLYHLRALFREGQFN

SEQ ID NO: 80 - protein, 113 aa, synthetic construct
PGPVPPSTALSELIEELINITQNQKAPLCNGSMVWSINPTAGMYCAALESLINVSGCSAIEKTQRMLSGFCPHKVAAGQFSSLHDKGSMIEVAQFVKDLLYHLRTLFREGQFN

SEQ ID NO: 81 - protein, 113 aa, synthetic construct
PGPVPPSTATRELIEELINITQNQKAPLCNGSMVWSINLTADMYCAALESLINVSGCSAIEKTQRMLSGFCPHKVSVGQFSSLHVRGSKIEVAQFVKDLLYHLRTLFREGQFN

SEQ ID NO: 82 - protein, 113 aa, synthetic construct
PGPVPPSTADIELIAELINITQNQKAPLCNGSMVWSINLTADMYCAALESLINVSGCSAIEKTQRMLSGFCPHKVSAGQFSSLHVKKTRIEVAQFVKDLLLHLKKLFKEGQFN

SEQ ID NO: 83 - protein, 113 aa, synthetic construct
PGPVPPSTAARELIEELVNITQNQKAPLCNGSMVWSINLTAGMYCAALESLINVSGCSAIEKTQRMLSGFCPHKVSAGQLSSLHVTGKRIEVAQFVKDLLNHLRALFKEGQFN

SEQ ID NO: 84 - protein, 113 aa, synthetic construct
PGPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFN

SEQ ID NO: 85 - protein, 113 aa, synthetic construct
PGPVPPSTALSELMEELVNITQNQKAPLCNGSMVWSINLTAGMYCAALESLINVSGCSAIEKTQRMLSGFCPHKVSAGQFSSLHVRDSKIEVAQFVKDLLNHLKALFKEGQFN

SEQ ID NO: 86 - protein, 112 aa, synthetic construct
GPVPPSTAFRELIEELVNITQNQKAPLCNGSMVWSINLTAGMYCAALESLINVSGCSAIEKTQRMLSGFCPHKVSPGQFSSLHVTNSRIEVAQFVKDLLNHLKALFKEGQYN

SEQ ID NO: 87 - protein, 112 aa, synthetic construct
GPVPPSTAHLELIEELINITQNQKAPLCNGSMVWSINLTAGMYCAALESLINVSGCSAIEKTQRMLSGFCPHKVSAGQFSSLHVKETRIEVAQFVKDLLNHLKTLFKEGQFN

SEQ ID NO: 88 - protein, 113 aa, synthetic construct
PGPVPPSTAHLELIEELINITQNQKAPLCNGSMVWSINPTAGMYCAALESLINVSGCSAIEKTQRMLSGFCPHKVSAGQFSSLHVMDTRIEVAQFVKDLLLHLKKLFKEGQFN

SEQ ID NO: 89 - protein, 113 aa, synthetic construct
PGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 90 - protein, 113 aa, synthetic construct
PGPVPPSTAHRELIEELVNITQNQKAPLCNGSMVWRINRTAGMYCAALESLINVSGCSAIEKTQRMLSGFCPHKVSAGQFSSLHVMDSRIEVAQFVKDLLNHLRALFKEGQFN

SEQ ID NO: 91 - protein, 113 aa, synthetic construct
PGPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFN

SEQ ID NO: 92 - protein, 113 aa, synthetic construct
PGPVPPSTALIELIEELINITQNQKAPLCNGSMVWSINLTAGMYCAALESLINVSGCSAIEKTQRMLSGFCPHKVSAGQFSSLHVKGSKIEVAQFVKDLLHHLRALMREGQFN

SEQ ID NO: 93 - protein, 113 aa, synthetic construct; identical to SEQ ID NO: 77

SEQ ID NO: 94 - protein, 113 aa, synthetic construct; identical to SEQ ID NO: 75

SEQ ID NO: 95 - protein, 113 aa, synthetic construct; identical to SEQ ID NO: 78

SEQ ID NO: 96 - protein, 113 aa, synthetic construct
PGPVPPSTALRELIEELINITQNQKAPLCNGSMVWSINLTAGMYCAALESLINVSGCSAIEKTQRMLSGFCPHKVSAGQFSSLHVTGSKIEVAQFVKDLLYHLRALFREGQFN

SEQ ID NO: 97 - protein, 113 aa, synthetic construct
PGPVPPSTALSELIEELINITQNQKAPLCNGSMVWSINLTAGMYCAALESLINVSGCSAIEKTQRMLSGFCPHKVSAGQFSSLHVKGSMIEVAQFVKDLLYHLRTLFREGQFN

SEQ ID NO: 98 - protein, 113 aa, synthetic construct
PGPVPPSTATRELIEELINITQNQKAPLCNGSMVWSINLTAGMYCAALESLINVSGCSAIEKTQRMLSGFCPHKVSAGQFSSLHVRGSKIEVAQFVKDLLYHLRTLFREGQFN

SEQ ID NO: 99 - protein, 113 aa, synthetic construct
PGPVPPSTADIELIEELINITQNQKAPLCNGSMVWSINLTAGMYCAALESLINVSGCSAIEKTQRMLSGFCPHKVSAGQFSSLHVKKTRIEVAQFVKDLLLHLKKLFKEGQFN

SEQ ID NO: 100 - protein, 113 aa, synthetic construct
PGPVPPSTAARELIEELVNITQNQKAPLCNGSMVWSINLTAGMYCAALESLINVSGCSAIEKTQRMLSGFCPHKVSAGQFSSLHVTGKRIEVAQFVKDLLNHLRALFKEGQFN

SEQ ID NO: 101 - protein, 113 aa, synthetic construct; identical to SEQ ID NO: 84

SEQ ID NO: 102 - protein, 113 aa, synthetic construct; identical to SEQ ID NO: 85

SEQ ID NO: 103 - protein, 113 aa, synthetic construct
PGPVPPSTAHLELIEELINITQNQKAPLCNGSMVWSINLTAGMYCAALESLINVSGCSAIEKTQRMLSGFCPHKVSAGQFSSLHVKETRIEVAQFVKDLLNHLKTLFKEGQFN

SEQ ID NO: 104 - protein, 113 aa, synthetic construct
PGPVPPSTAHLELIEELINITQNQKAPLCNGSMVWSINLTAGMYCAALESLINVSGCSAIEKTQRMLSGFCPHKVSAGQFSSLHVMDTRIEVAQFVKDLLLHLKKLFKEGQFN

SEQ ID NO: 105 - protein, 113 aa, synthetic construct; identical to SEQ ID NO: 89

SEQ ID NO: 106 - protein, 113 aa, synthetic construct
PGPVPPSTAHRELIRELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 107 - protein, 113 aa, synthetic construct
PGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGRFN

SEQ ID NO: 108 - protein, 113 aa, synthetic construct
PGPVPPSTAHRELIEELVNITQNQKAPLCNGSMVWSINLTAGMYCAALESLINVSGCSAIEKTQRMLSGFCPHKVSAGQFSSLHVMDSRIEVAQFVKDLLNHLRALFKEGQFN

SEQ ID NO: 109 - protein, 113 aa, synthetic construct
PGPVPPSTAARELIEELFNITQNQKAPLCNGSMVWSINLTAGMYCAALESLINVSGCSAIEKTQRMLSGFCPHKVSAGQFSSLHVKKTRIEVAQFVKDLLIHLRKLFKEGQFN

SEQ ID NO: 110 - protein, 113 aa, synthetic construct
PGPVPPSTAVRALIEELINITQNQKAPLCNGSMVWSINLTAGMYCAALESLINVSGCSAIEKTQDMLSGFCPHKVSAGQFSSLHVRSSKIEVAQFVKDLLFHLRTLFREGQFN

SEQ ID NO: 111 - protein, 113 aa, synthetic construct
PGPVPPSTAHRELIEELVNITQNQKAPLCNGSMVWSINRTAGMYCAALESLINVSGCSAIEKTQRMLSGFCPHKVSAGQFSSLHVTGRKIEVAQFVKDLLLHLKKLFKEGQFN

SEQ ID NO: 112 - protein, 113 aa, synthetic construct; identical to SEQ ID NO: 74

SEQ ID NO: 113 - protein, 114 aa, synthetic construct
MPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 114 - protein, 117 aa, synthetic construct
MYCAALESLINVSGCSAIEKTQRMLSGFCPHKVSAGQFSSLHVRDTKIEVAQFVKDLLLHLKKLFREGQFNGGSGPGPVPPSTALRELIEELVNITQNQKAPLCNGSMVWSINLTAG

SEQ ID NO: 115 - protein, 118 aa, synthetic construct
MYCAALESLINVSGCSAIEKTQRMLSGFCPHKVSAGQFSSLHVRDTKIEVAQFVKDLLLHLKKLFREGQFNGGSGMPGPVPPSTALRELIEELVNITQNQKAPLCNGSMVWSINLTAG

SEQ ID NO: 116 - protein, 117 aa, synthetic construct
MYCAALESLINVSGCSAIEKTQDMLSGFCPHKVSAGQFSSLHVRSSKIEVAQFVKDLLFHLRTLFREGQFNGGSGPGPVPPSTAVRALIEELINITQNQKAPLCNGSMVWSINLTAG

SEQ ID NO: 117 - protein, 118 aa, synthetic construct
MYCAALESLINVSGCSAIEKTQDMLSGFCPHKVSAGQFSSLHVRSSKIEVAQFVKDLLFHLRTLFREGQFNGGSGMPGPVPPSTAVRALIEELINITQNQKAPLCNGSMVWSINLTAG

SEQ ID NO: 118 - protein, 113 aa, synthetic construct
PGPVPPSTAVRELIEELINITQNQKAPLCNGSMVWSINRTAGMYCAALESLINVSGCSAIEKTQRMLSGFCPHKVSAGQFSSLHVRSSKIEVAQFVKDLLFHLRTLFREGQFN

SEQ ID NO: 119 - protein, 113 aa, synthetic construct; identical to SEQ ID NO: 118

SEQ ID NO: 120-122 - skipped (3 entries)

SEQ ID NO: 123 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 124 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 125 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 126 - protein, 355 aa, synthetic construct
PGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFNGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 128 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINRTAGMYCAALESLINVSGCSAIEKTQRMLSGFCPHKVSAGQFSSLHVTGRKIEVAQFVKDLLLHLKKLFKEGQFN

SEQ ID NO: 129 - protein, 713 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSPGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 130 - protein, 354 aa, synthetic construct; identical to SEQ ID NO: 128

SEQ ID NO: 131 - protein, 344 aa, synthetic construct
APPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGSGSPGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 132 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELINITQNQKAPLCNGSMVWSINLTAGMYCAALESLINVSGCSAIEKTQDMLSGFCPHKVSAGQFSSLHVRSSKIEVAQFVKDLLFHLRTLFREGQFN

SEQ ID NO: 133 - protein, 354 aa, synthetic construct
PGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGRFNGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGRFN

SEQ ID NO: 136 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELINITQNQKAPLCNGSMVWSINLTAGMYCAALESLINVSGCSAIEKTQDMLSGFCPHKVSAGQFSSLHVRSSKIEVAQFVKDLLFHLRTLFREGRFN

SEQ ID NO: 137 - protein, 354 aa, synthetic construct
PGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFNGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - skipped

SEQ ID NO: 139 - protein, 126 aa, synthetic construct
HHHHHHENLYFQGPGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 140 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELHNITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 141 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELINITQNQKAPLCNGRMVWSINRTAGMYCAALESLINVSGCSAIEKTQDMLSGFCPHKVSAGQFSSLHVRSSKIEVAQFVKDLLFHLRTLFREGQFN

SEQ ID NO: 142 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELHNITQNQKAPLCNGSMVWSINRTAGMYCAALESLINVSGCSAIEKTQDMLSGFCPHKVSAGQFSSLHVRHSKIEVAQFVKDLLFHLRTLFREGQFN

SEQ ID NO: 143 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELINITQNQKAPLCNGSMVWSINRTAGMYCAALESLINVSGCSAIEKTQDMLSGFCPHKVSAGQFSSLHVRSSKIEVARFVKDLLFHLRTLFREGQFN

SEQ ID NO: 144 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLWREGQFN

SEQ ID NO: 145 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGEEEKRKREEEEGSPGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 146 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGEAAAKEAAAKEAAAKPGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 147 - protein, 713 aa, synthetic construct
PGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFNGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 148 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRIIEVAQFVKDLLLHLKKLFKEGQFN

SEQ ID NO: 149 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIRELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 150 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLRLHLKKLFKEGQFN

SEQ ID NO: 151 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPRTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 152 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKIRVAQFVKDLLLHLKKLFKEGQFN

SEQ ID NO: 153 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIRELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 154 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 155 - protein, 355 aa, synthetic construct
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 157 - protein, 113 aa, synthetic construct; identical to SEQ ID NO: 89

SEQ ID NO: 158 - protein, 113 aa, synthetic construct; identical to SEQ ID NO: 107

SEQ ID NO: 159 - protein, 119 aa, synthetic construct
MTAGMYCAALESLINVSGCSAIEKTQRMLSGFCPHKVSAGQFSSLHVTGRKIEVAQFVKDLLLHLKKLFKEGQFNGGNGGPGPVPPSTAHRELIRELVNITQNQKAPLCNGSMVWSINL

SEQ ID NO: 160 - protein, 119 aa, synthetic construct
MTAGMYCAALESLINVSGCSAIEKTQRMLSGFCPHKVSAGQFSSLHVTGRKIEVAQFVKDLLLHLKKLFKEGRFNGGNGGPGPVPPSTAHRELIRELVNITQNQKAPLCNGSMVWSINL

SEQ ID NO: 161 - protein, 469 aa, synthetic construct
MTAGMYCAALESLINVSGCSAIEKTQRMLSGFCPHKVSAGQFSSLHVTGRKIEVAQFVKDLLLHLKKLFKEGQFNGGNGGPGPVPPSTAHRELIRELVNITQNQKAPLCNGSMVWSINLASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 162 - protein, 469 aa, synthetic construct
MTAGMYCAALESLINVSGCSAIEKTQRMLSGFCPHKVSAGQFSSLHVTGRKIEVAQFVKDLLLHLKKLFKEGRFNGGNGGPGPVPPSTAHRELIRELVNITQNQKAPLCNGSMVWSINLASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 163 - protein, 464 aa, synthetic construct
GPGPVPPSTAHRELIRELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFNASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 164 - protein, 464 aa, synthetic construct
GPGPVPPSTAHRELIRELVNITQNQKAPLCNGSMVWSINLTAGMYCAALESLINVSGCSAIEKTQRMLSGFCPHKVSAGQFSSLHVTGRKIEVAQFVKDLLLHLKKLFKEGRFNASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 165-172 - skipped (8 entries)

SEQ ID NO: 173 - protein, 597 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFSQSIISTLT

SEQ ID NO: 174 - protein, 450 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 175 - protein, 218 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 590 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFSQSIISTLT

SEQ ID NO: 177 - protein, 443 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 178 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 590 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCQSIISTLT

SEQ ID NO: 180 - protein, 597 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFCQSIISTLT

SEQ ID NO: 181 - protein, 590 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFYMPKKATELKHLQCLEEALKPLEEVLNLAQSKNFHFDPRDVVSNINVFVLELKGSETTFMCEYADETATIVEFLNRWITFSQSIISTLT

SEQ ID NO: 182 - protein, 590 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 183 - protein, 597 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 184 - protein, 597 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCTSIISTLT

SEQ ID NO: 185-193 - skipped (9 entries)

SEQ ID NO: 194 - protein, 577 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 195 - protein, 570 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 196 - protein, 577 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 197 - protein, 570 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 198 - protein, 577 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIRELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 199-300 - skipped (102 entries)

SEQ ID NO: 301 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 302 - protein, 133 aa, Homo sapiens [sequence omitted]

SEQ ID NO: 303 - protein, 169 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 304 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 305-499 - skipped (195 entries)

SEQ ID NO: 500 - protein, 573 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 501 - protein, 446 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 502 - protein, 214 aa, synthetic construct
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 503 - protein, 457 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 504 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 505 - protein, 107 aa, synthetic construct [sequence omitted]

SEQ ID NO: 506 - protein, 457 aa, synthetic construct; identical to SEQ ID NO: 503

SEQ ID NO: 507 - protein, 330 aa, synthetic construct; identical to SEQ ID NO: 504

SEQ ID NO: 508 - protein, 107 aa, synthetic construct [sequence omitted]

SEQ ID NO: 509-999 - skipped (491 entries)

SEQ ID NO: 1000 - protein, 5 aa, synthetic construct
GGGGS

SEQ ID NO: 1001 - protein, 10 aa, synthetic construct
GGGGSGGGGS

SEQ ID NO: 1002 - protein, 15 aa, synthetic construct
GGGGSGGGGSGGGGS

SEQ ID NO: 1003 - protein, 20 aa, synthetic construct
GGGGSGGGGSGGGGSGGGGS

SEQ ID NO: 1004 - protein, 20 aa, synthetic construct
GGGGSGGGGSGGGGSGGGGS

SEQ ID NO: 1005 - protein, 10 aa, synthetic construct
SGARYRWLTA

SEQ ID NO: 1006 - protein, 10 aa, synthetic construct
SGRSYAILTA

SEQ ID NO: 1007 - protein, 14 aa, synthetic construct
SRSGRSPAIFTATG

SEQ ID NO: 1008 - protein, 14 aa, synthetic construct
GSSGRSPAIFTAGS

SEQ ID NO: 1009 - protein, 10 aa, synthetic construct
SGFIANPVTA

SEQ ID NO: 1010-1049 - skipped (40 entries)

SEQ ID NO: 1050 - protein, 153 aa, synthetic construct [sequence omitted]

SEQ ID NO: 1051 - protein, 130 aa, synthetic construct
MHKCDITLQEIIKTLNSLTEQKTLCTELTVTDIFAASKDTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 1052 - protein, 128 aa, synthetic construct
KCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMKEKFRKCSS

SEQ ID NO: 1053 - protein, 134 aa, synthetic construct
MDTTEKETFCRAATVLRQFYSHHEKDTRCLGATAQQFHRHKQLIRFLKRLDRNLWGLAGLNSCPVKEANQSTLENFLERLRVIMQSKWFKCGAGGNGGHKCDITLQEIIKTLNSLTEQKTLCTELTVTDIFAAS

SEQ ID NO: 1054 - protein, 134 aa, synthetic construct
MDTTEKETFCRAATVLRQFYSHHEKDTRCLGATAQQFHRHKQLIRFLKRLDRNLWGLAGLNSCPVKEANQSTLENFLERLKTIMREKYSKCSSGGNGGHKCDITLQEIIKTLNSLTEQKTLCTELTVTDIFAAS

SEQ ID NO: 1055 - protein, 134 aa, synthetic construct
MDTTEKETFCRAATVLRQFYSHHEKDTRCLGATAQQFHRHKQLIRFLKRLDRNLWGLAGLNSCPVKEANQSTLENFLERLKTIMKEKFRKCSSGGNGGHKCDITLQEIIKTLNSLTEQKTLCTELTVTDIFAAS

SEQ ID NO: 1056 - protein, 133 aa, synthetic construct
MDTTEKETFCRAATVLRQFYSHHEKDTRCLGATAQQFHRHKQLIRFLKRLDRNLWGLAGLNSCPVKEANQSTLENFLERLKTIMKEKFKCSSGGNGGHKCDITLQEIIKTLNSLTEQKTLCTELTVTDIFAAS

SEQ ID NO: 1057 - protein, 485 aa, synthetic construct
MDTTEKETFCRAATVLRQFYSHHEKDTRCLGATAQQFHRHKQLIRFLKLRDRNLWGLAGLNSCPVKEANQSTLENFLERLKTIMREKYSKCSSGGNGGHKCDITLQEIIKTLNSLTEQKTLCTELTVTDIFAASKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 1058 - protein, 4 aa, synthetic construct
GFLG

SEQ ID NO: 1059 - protein, 6 aa, synthetic construct
RRALAL

SEQ ID NO: 1060 - protein, 4 aa, synthetic construct
GFLG

SEQ ID NO: 1061 - protein, 4 aa, synthetic construct
ALAL

SEQ ID NO: 1062 - protein, 5 aa, synthetic construct
  VARIANT 1..5: note = The entire sequence of amino acids 1-5 can be repeated one to ten times.
GGGGS

SEQ ID NO: 1063 - protein, 6 aa, synthetic construct
HHHHHH

SEQ ID NO: 1064 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1065 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 1066 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 1067 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1068 - protein, 354 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGPVPPSTAHRELIEELVNITQNQKAPLCNGSMVWSINLTAGMYCAALESLINVSGCSAIEKTQRMLSGFCPHKVSAGQFSSLHVTGRKWEVAQFVKDLLLHLKKLFKEGQFN